(19) United States
(12) Patent Application Publication
(10) Pub. No.: US 2011/0257251 A1
(43) Pub. Date: Oct. 20, 2011

(54) TELOMERASE INHIBITORS AND METHODS OF USE THEREOF

(75) Inventors: Lourdes Gude-Rodriguez, Madrid (ES); Gregory L. Verdine, Newton, MA (US); Shaunna Syu-Mei Stanton, Cambridge, MA (US)

(73) Assignee: PRESIDENTS AND FELLOWS OF HARVARD COLLEGE, Cambridge, MA (US)

(21) Appl. No.: 13/123,123

(22) PCT Filed: Oct. 7, 2009
(86) PCT No.: PCT/US09/59867

§ 371 Date: Jul. 5, 2011

Related U.S. Application Data
(60) Provisional application No. 61/103,430, filed on Oct. 7, 2008.

(51) Int. Cl. A61K 31/7105; C12N 9/99; A61P 35/00; C07H 21/02

(52) U.S. Cl. 514/44 R; 536/23.1; 435/184

(57) ABSTRACT

One object of the present invention is to provide methods and compositions for inhibiting human telomerase, by providing inhibitors that bind to the CR4-CR5 or pseudoknot/template domains of the RNA component of human telomerase.

### CROSS REFERENCE TO RELATED APPLICATIONS

This application claims the benefit under 35 U.S.C. §119(e) of U.S. Provisional Patent Application Ser. No. 61/103,430 filed on Oct. 7, 2008, the contents of which are incorporated herein in their entirety by reference.

### GOVERNMENT SUPPORT

This invention was made with Government support under Training Grant No. 5 T32 GM007598 awarded by the Molecular and Cell Biology Department (MCB) of the National Institutes of Health (NIH). The Government has certain rights in the invention.

### FIELD OF THE INVENTION

The present invention relates to compositions and methods for the treatment of cancer and other proliferative disorders. More specifically, the invention relates to telomerase inhibitors and their uses therein.

### BACKGROUND OF THE INVENTION

During the last few years, the field of cancer drug discovery has experienced notable advances in terms of understanding the crucial requirements in the search for selective and efficient drugs, as well as the rationale used for the selection of molecular targets (S. L. Mooberry, Drug Discovery Handbook. Wiley-Interscience 1343-1368 (2005)). Small-molecule based ligands that can fit into well-defined hydrophobic pockets of proteins are still regarded as classical drug options, and proteins the most prevalent therapeutic targets within what has been termed the ‘druggable’ genome (A. L. Hopkins, Nat. Rev. Drug Discovery 1, 727-730 (2002)). However, considerable attention is currently being paid to the search for novel compounds, chemistries, and approaches that can adequately target other molecular key players besides proteins, some of them traditionally viewed as cumbersome, impractical, or simply ‘undruggable’. In particular, RNA has been relegated for many years as a mere carrier of genetic information, despite its many roles in diverse cellular processes (e.g., ribozymes, riboswitches, miRNAs). The intrinsic possibilities for therapeutic intervention, including but not limited to the possibility of controlling gene expression by using traditional (antisense) and recent (RNAi) approaches, have resulted in a growing interest for RNA structure and function. Although challenging, efforts aimed at targeting RNA with small molecules hold great promise, and the inherently flexible and complex structure of RNA could in principle be used as a basis for rational design of novel strategies aimed at disrupting its function (J. R. Thomas, Chem. Rev. 108, 1171-1224 (2008)). This is expected to be especially relevant not only in targeting messenger RNAs, but also in targeting other well-structured, non-coding RNAs that play essential roles in a cellular context. Short oligonucleotides have been previously reported to possess relevant properties in the RNA targeting arena. ODMiR (Oligonucleotide Directed Misfolding of RNA), for example, has proven to be an effective method for the inhibition of group I introns and E. Coli RNase P (J. L. Childs, Proc. Natl. Acad. Sci. USA 99, 11091-11096 (2002); J. L. Childs, RNA 9, 1437-1445 (2003)).

Telomerase is a specialized ribonucleoprotein composed of two essential components, a reverse transcriptase protein subunit (hTERT), and an RNA component (hTR) (J. Feng, Science 269, 1236-1241 (1995); T. M. Nakamura, Science 277, 911-912 (1997)), as well as several associated proteins. It directs the synthesis of telomeric repeats (5′-TTAGGG-3′) at chromosome ends, using a short sequence within the RNA component as a template. Telomerase is considered to be an almost universal marker for human cancer, its effect on telomere length playing a crucial role in evading replicative senescence. Indeed, whereas in most normal somatic cells telomerase activity is repressed, it has been found that it is activated in approximately 90% of human tumors (J. W. Shay, Eur. J. Cancer 33, 787-791 (1991); N. W. Kim, Science 266, 2011-2015 (1994)).

### SUMMARY OF THE INVENTION

One object of the present invention is to provide methods and compositions for inhibiting human telomerase, by providing inhibitors that bind to the CR4-CR5 domain of the RNA component of human telomerase.

Accordingly, in one aspect, a telomerase inhibitor comprising a nucleic acid or analog thereof that binds to the CR4-CR5 domain of the RNA component of human telomerase is provided. In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. In a preferred embodiment, the telomerase inhibitor binds to the J5/J6 loop of the CR4-CR5 domain of the RNA component of human telomerase.

In one embodiment, the nucleic acid or analog thereof that binds to the CR4-CR5 domain of the RNA component of human telomerase comprises a binding sequence length of 4-20 nucleotides. In another embodiment, the nucleic acid or analog thereof comprises a binding sequence length of 6-14 nucleotides. In another embodiment, the nucleic acid or nucleic acid analog thereof comprises a binding sequence length of about 10 nucleotides. In another embodiment, the nucleic acid or analog thereof has a binding sequence length of 10 nucleotides. In another embodiment, the nucleic acid or analog thereof comprises a binding sequence length of 8 nucleotides.

In one embodiment, the telomerase inhibitor that binds to the CR4-CR5 domain of the RNA component of human telomerase is selected from the group consisting of SEQ ID NO: 1-SEQ ID NO: 10. In one embodiment, the telomerase inhibitor that binds to the CR4-CR5 domain of the RNA component of human telomerase comprises SEQ ID NO: 1 or SEQ ID NO: 2.

Another aspect of the invention provides a method of inhibiting telomerase activity comprising contacting a telomerase with a nucleic acid or analog thereof, which binds to the CR4-CR5 domain of the RNA component of human telomerase. In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. In one embodiment, the telomerase inhibitor binds to the J5/J6 loop of the CR4-CR5 domain of the RNA component of human telomerase.

In one embodiment, the nucleic acid or analog thereof that binds to the CR4-CR5 domain of the RNA component of human telomerase comprises a binding sequence length of 4-20 nucleotides. In another embodiment, the nucleic acid or analog thereof comprises a binding sequence length of 6-14 nucleotides. In another embodiment, the nucleic acid or analog thereof comprises a binding sequence length of about 10 nucleotides. In another embodiment, the nucleic acid or analog thereof has a binding sequence length of 10 nucleotides. In another embodiment, the nucleic acid or analog thereof comprises a binding sequence length of about 8 nucleotides.

In one embodiment, the telomerase inhibitor that binds to the CR4-CR5 domain of the RNA component of human telomerase is selected from the group consisting of SEQ ID NO: 1-SEQ ID NO: 10. In a preferred embodiment, the telomerase inhibitor that binds to the CR4-CR5 domain of the RNA component of human telomerase comprises SEQ ID NO: 1 or SEQ ID NO: 2.

Another aspect provides a method of inhibiting telomerase activity in a cell, the method comprising contacting a cell with a nucleic acid or analog thereof, which binds to the CR4-CR5 domain of the RNA component of human telomerase.

In one embodiment, the cell is contacted in vitro. In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. In a preferred embodiment, the telomerase inhibitor binds to the J5/J6 loop of the CR4-CR5 domain of the RNA component of human telomerase.

In one embodiment, the nucleic acid or analog thereof that binds to the CR4-CR5 domain of the RNA component of human telomerase comprises a binding sequence length of 4-20 nucleotides. In another embodiment, the nucleic acid or analog thereof comprises a binding sequence length of 6-14 nucleotides. In another embodiment, the nucleic acid or analog thereof comprises a binding sequence length of about 10 nucleotides. In another embodiment, the nucleic acid or analog thereof has a binding sequence length of 10 nucleotides. In another embodiment, the nucleic acid or analog thereof comprises a binding sequence length of about 8 nucleotides.

In one embodiment, the telomerase inhibitor that binds to the CR4-CR5 domain of the RNA component of human telomerase is selected from the group consisting of SEQ ID NO: 1-SEQ ID NO: 10. In a preferred embodiment, the telomerase inhibitor that binds to the CR4-CR5 domain of the RNA component of human telomerase comprises SEQ ID NO: 1 or SEQ ID NO: 2.

Another aspect provides a method of treating a proliferative disorder in a subject in need thereof, comprising administering to the subject an effective amount of a telomerase inhibitor comprising a nucleic acid or analog thereof that binds to the CR4-CR5 domain of the RNA component of human telomerase.

In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. In a preferred embodiment, the telomerase inhibitor binds to the J5/J6 loop of the CR4-CR5 domain of the RNA component of human telomerase.

In one embodiment, the nucleic acid or nucleic acid analog thereof that binds to the CR4-CR5 domain of the RNA component of human telomerase comprises a binding sequence length of 4-20 nucleotides. In another embodiment, the nucleic acid or analog thereof comprises a binding sequence length of 6-14 nucleotides. In another embodiment, the nucleic acid or analog thereof comprises a binding sequence length of about 10 nucleotides. In another embodiment, the nucleic acid or analog thereof has a binding sequence length of 10 nucleotides. In another embodiment, the nucleic acid or analog thereof comprises a binding sequence length of about 8 nucleotides.

In one embodiment, the telomerase inhibitor that binds to the CR4-CR5 domain of the RNA component of human telomerase is selected from the group consisting of SEQ ID NO: 1-SEQ. ID NO: 10. In a preferred embodiment, the telomerase inhibitor that binds to the CR4-CR5 domain of the RNA component of human telomerase comprises SEQ ID NO: 1 or SEQ ID NO: 2. In one embodiment, the proliferative disorder being treated in the subject is a cancer.

In another aspect, a therapeutic composition comprising a telomerase inhibitor and a pharmaceutically acceptable carrier is provided, where the telomerase inhibitor comprises a nucleic acid or analog thereof that binds to the CR4-CR5 domain of the RNA component of human telomerase.

In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. In a preferred embodiment, the telomerase inhibitor binds to the J5/J6 loop of the CR4-CR5 domain of the RNA component of human telomerase.

In one embodiment, the nucleic acid or analog thereof that binds to the CR4-CR5 domain of the RNA component of human telomerase comprises a binding sequence length of 4-20 nucleotides. In another embodiment, the nucleic acid or analog thereof comprises a binding sequence length of 6-14 nucleotides. In another embodiment, the nucleic acid or analog thereof comprises a binding sequence length of about 10 nucleotides. In another embodiment, the nucleic acid or analog thereof has a binding sequence length of 10 nucleotides. In another embodiment, the nucleic acid or analog thereof comprises a binding sequence length of about 8 nucleotides.

In one embodiment, the telomerase inhibitor that binds to the CR4-CR5 domain of the RNA component of human telomerase is selected from the group consisting of SEQ ID NO: 1-SEQ. ID NO: 10. In one embodiment, the telomerase inhibitor that binds to the CR4-CR5 domain of the RNA component of human telomerase comprises SEQ ID NO: 1 or SEQ ID NO: 2.

Another object of the present invention is to provide methods and compositions for inhibiting human telomerase, by providing inhibitors that bind to the pseudoknot/template domain of the RNA component of human telomerase.

Accordingly, one aspect provides a telomerase inhibitor comprising a ribonucleic acid molecule or analog thereof that binds to the pseudoknot/template domain of the RNA component of human telomerase, where the ribonucleic acid molecule or ribonucleic acid analog thereof comprises a binding sequence selected from the group consisting of SEQ ID NO: 12-SEQ. ID NO: 45. In one embodiment, the telomerase inhibitor is selected from the group consisting SEQ ID NO: 19-SEQ ID NO: 24; SEQ ID NO: 39; SEQ ID NO: 44 and SEQ. ID NO: 45. In another embodiment, the telomerase inhibitor binding sequence comprises SEQ. ID NO: 20.

In one embodiment a method of inhibiting telomerase activity in a cell is provided, comprising contacting a cell with a ribonucleic acid molecule or analog thereof, which binds to the pseudoknot/template domain of the RNA component of human telomerase, where the ribonucleic acid molecule or ribonucleic acid analog thereof comprises a binding sequence selected from the group consisting of SEQ ID NO: 12-SEQ. ID NO: 45. In one embodiment, the telomerase inhibitor is selected from the group consisting SEQ ID NO: 19-SEQ ID NO: 24; SEQ ID NO: 39; SEQ ID NO: 44 and SEQ. ID NO: 45. In another embodiment, the telomerase inhibitor binding sequence comprises SEQ. ID NO: 20.

Another aspect provides a method of treating a proliferative disorder in a subject in need thereof, comprising administering to the subject an effective amount of a telomerase inhibitor, the telomerase inhibitor comprising a ribonucleic acid molecule or analog thereof that binds to the pseudoknot/template domain of the RNA component of human telomerase, and where said wherein the ribonucleic acid molecule or analog thereof comprises a binding sequence selected from the group consisting of SEQ ID NO: 12-SEQ. ID NO: 45. In one embodiment, the telomerase inhibitor is selected from the group consisting SEQ ID NO: 19-SEQ ID NO: 24; SEQ ID NO: 39; SEQ ID NO: 44 and SEQ. ID NO: 45. In another embodiment, the telomerase inhibitor binding sequence comprises SEQ. ID NO: 20. In one embodiment, the proliferative disorder is a cancer.

Another aspect provides a therapeutic composition comprising a telomerase inhibitor and a pharmaceutically acceptable carrier, where the telomerase inhibitor comprises a nucleic acid or analog thereof that binds to the pseudoknot/template domain of the RNA component of human telomerase, and where the ribonucleic acid molecule or analog thereof comprises a binding sequence selected from the group consisting of SEQ ID NO: 11-SEQ ID NO: 45. In one embodiment, the telomerase inhibitor is selected from the group consisting SEQ ID NO: 19-SEQ ID NO: 24; SEQ ID NO: 39; SEQ ID NO: 44 and SEQ. ID NO: 45. In another embodiment, the telomerase inhibitor binding sequence comprises SEQ. ID NO: 20.

Methods or compositions “comprising” one or more recited elements may include other elements not specifically recited, whether essential or not. For example, a telomerase inhibitor that comprises a nucleic acid or analog therein encompasses both the nucleic acid sequence and the nucleic acid sequence as a component of a larger nucleotide sequence, such as a vector or plasmid. By way of further example, a composition that comprises elements A and B also encompasses a composition consisting of A, B and C. The terms “comprising” means “including principally, but not necessarily solely”. Furthermore, variation of the word “comprising”, such as “comprise” and “comprises”, have correspondingly varied meanings.

As used herein, the term “consisting essentially of” refers to those elements required for a given embodiment. The term permits the presence of additional elements that do not materially affect the basic and novel or functional characteristic(s) of that embodiment of the invention, and as such, is intended to mean “including principally, but not necessarily solely at least one.”

As used herein, the term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” include one or more methods, and/or steps of the type described herein and/or which will become apparent to those persons skilled in the art upon reading this disclosure and so forth. Other than in the operating examples, or where otherwise indicated, all numbers expressing quantities of ingredients or reaction conditions used herein should be understood as modified in all instances by the term “about.” The term “about” when used in connection with percentages can mean±1%. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### BRIEF DESCRIPTION OF THE DRAWINGS

FIGS. 1A-1C provide an overview of the RIPtide microarray technology. FIG. 1A shows a schematic representation of a RIPtide microarray. FIG. 1B shows the structure of the 2′-O-methyl RIPtides and a polar polyethylenglycol linker. FIG. 1C shows the RIPtide array format. In this example, each chip contains a total 87,296 RIPtide sequences. The number of RIPtides per N-mer family is indicated (N=4, 5, 6, 7, 8).

FIGS. 2A-2I depicts the fabrication of oligo (2′-O-Me-ribonucleotide) RIPtide microarrays using a photo-imageable polymer film containing a photo-acid generator (PAG) (ref. 13). FIG. 2A shows how fused silica substrates are cleaned, and treated with a suitable silane to introduce a surface layer containing covalently bonded hydroxyalkyl groups. FIG. 2B shows how using standard oligonucleotide synthesis protocols, the surface hydroxyl sites are extended with a PEG molecular spacer protected at the distal end with a DMT group. FIG. 2C shows how the PAG film is then applied to the substrate, and exposed with a photolithographic mask to generate a pattern of photo-generated acid in the film with feature spacing of 17.5 microns (FIG. 2D). FIG. 2E shows how the photo-generated acid removes the DMT protecting groups from the hydroxyl sites in the imaged regions. FIG. 2F shows how the PAG film is removed, and FIG. 2G shows how the substrate is exposed to a solution of activated 5′-O-DMT-2′-O-Me-ribonucleoside phosphoramidite, followed by standard capping and oxidizer reagents. This couples a first nucleotide in regions of the substrate exposed in step d (eg., 2′-OMe-A). FIGS. 2H-2I show how the steps depicted in FIGS. 2C-2G are repeated to complete the remaining sequences of the array (three additional cycles shown for C, G, and U). After completion of all sequences, substrates are processed through final deprotection, dicing, and packaging of the individual arrays.

FIGS. 3A-3B depict a schematic diagram with the sequences and the secondary structures of the hTR constructs used. FIG. 3A shows the engineered hTR pseudoknot constructs (PKWT and PKWT-1, top; SEQ ID NO: 67 and SEQ ID NO:68, respectively, in order of appearance) and sequence of the template/pseudoknot domain (SEQ ID NO: 69, bottom) of hTR. Capital letters correspond to residues ≧80% conserved in vertebrates. FIG. 3B depicts the secondary structure model of hTR, adapted from 31, including a schematic representation of the different RNA constructs screened with the RIPtide platform.

FIG. 4A shows the cluster profiles of PKWT and PKWT-1 corresponding to a 100 nM, 1 h incubation. Number of hits (out of 100) are represented (y-axis) versus nucleotide position of the screened RNA construct (x-axis, expressed relative to hTR sequence). FIG. 4B shows the rank of top (more intense) 10 RIPtide hits and K_{d} values determined with unlabeled PKWT-1. FIG. 4B discloses SEQ ID NO: 28-SEQ ID NO: 30, SEQ ID NO: 11 and SEQ ID NO: 31-SEQ ID NO: 36, respectively, in order of appearance. FIG. 4C shows the cluster profiles of PK123 and PK159 using standard (100 nM, 1 h) incubation conditions. The hTR sequence nucleotides to which RIPtide aligns is represented on the x-axis. FIG. 4D provides a summary of results from 2′-O-methyl screening of the Template/Pseudoknot domain of hTR. In the second column, the consensus identified RIPtide sequence is indicated, with X representing regions with variable length. In the third column, the nucleotide position of hTR that aligns with the middle (4^{th}) position of the RIPtide 5′-3′ is shown. n.d.=not determined. Data represent average±s.d. of three independent samples. FIG. 4D discloses SEQ ID NO: 46-SEQ ID NO: 51, respectively, in order of appearance.

FIG. 5 shows the effect of RNA incubation time on PKWT-1 clustering profile. Lower concentrations of the RNA target were employed at higher incubation times, so as to avoid fluorescence saturation. PKWT-1 sequence numbering corresponds to nucleotide position (nt) in the synthetic construct, and not to the hTR sequence. Hits in Cluster II showed a greater tendency to accumulate over time than hits in Cluster I.

FIGS. 6A-6C depict 2′-O-Me RIPtide mapping of the pseudoknot domain of hTR. FIG. 6A shows the dissociation constants between selected RIPtides and unlabeled full-length hTR, expressed in nanomolar units. Clusters are coded according to shades of grey. FIG. 6A discloses Clusters I-1, I-2, II-1, II-2, I-3, III-1, III-2, IV-1, IV-21, V-2 and V-3 as SEQ ID NO: 37-SEQ ID NO: 38, SEQ ID NO: 28, SEQ ID NO: 11, SEQ ID NO: 12, SEQ ID NO: 14, SEQ ID NO: 15, SEQ ID NO: 39, SEQ ID NO: 19, SEQ ID NO: 25, and SEQ ID NO: 26, respectively. FIG. 6B shows targetable regions in the template/pseudoknot domain of hTR and indicated on the secondary structure of the hTR core. Bases indicated in bold represent the mutation sites for the fluorescence polarization studies. Capital letters correspond to residues ≧80% conserved in vertebrates. Data represent average±s.d. of three independent samples and are representative for two independent experiments. FIG. 6B discloses SEQ ID NO: 69. FIG. 6C depicts bar graphs with RIPtide-hTR K_{d} values, colored according the relative RIPtide-hTR binding affinity.

FIGS. 7A-7D show compensatory mutation studies showing the FP binding curves for hTR-RIPtide interactions. RIPtides were FAM-labeled at the 3′ end. RIPtide binding sites were confirmed by FP assays in the presence of mutated full length hTR, mutated RIPtides, or both. Binding profiles of: WT hTR and RIPtides are shown in FIG. 7A; mutant hTR and ‘wild-type’ RIPtides are shown in FIG. 7B; WT-hTR and ‘mutant’ RIPtides are shown in FIG. 7C; and mutant hTR and mutant RIPtides are shown in FIG. 7D. Chosen hTR mutation sites are shown in FIG. 6 for each identified cluster. RIPtides were mutated at the two central bases. All mutations involved substitution of the two consecutive bases to their complementary bases. Overall, the figure shows that no increase in polarization was observed where mutations were introduced in one of the binding partners. However, binding of several mutant RIPtides to hTR was restored in some cases by the introduction of compensatory mutations into the putative binding site on hTR. Polarization shown in FIG. 7B-7D was renormalized with respect to the WT-hTR, RIPtide situation reflected in graph a. Points, mean; bars, s.d. Experiments were preformed in triplicate.

FIG. 8A shows selected RIPtides with anti-telomerase activity. PD=phosphodiester backbone, PS=phosphorothioate backbone, 2′-OMe=2′-O-methyl. Lowercase font indicates the presence of a phosphorothioate linkage. IC₅₀ and K_{d} values are reported in nM. 60 μM RIPtide was added after PCR to control for PCR inhibition. 2′-O-Me RIPtides derived from sequence but containing mismatches were used to assess sequence-specificity effects. Mismatches are indicated in italics: GGUGCAAGGC (SEQ ID NO: 52), GGUGCCAGGC (SEQ ID NO: 53), and GCUGCAACGC (SEQ ID NO: 54) (PD), and GGUGCCAGGC (SEQ ID NO: 53) (fully PS substitution). FIG. 8A discloses IV-3, IV-4 and IV-5 as SEQ ID NO: 20. FIG. 8B shows Dose-response inhibition of telomerase by RIPtide IV-3. FIG. 8C shows a TRAP gel (single experiment) representing inhibition of telomerase activity by RIPtide IV-3 in HeLa cell extracts. Lane 1: 60 μM, lane 2: 6 μM, lane 3: 600 nM, lane 4: 60 nM, lane 5: 6 nM, lane 7: 600 pM, lane 8: 60 pM, lane 9: 6 pM, lane 10: 0.6 pM. FIG. 8D depicts a bar graph with telomerase inhibition by selected RIPtides IV-3 and IV-5 in DU145 cells. Cells were treated with 165 nM of RIPtide for 24 h, in triplicate. Lipofectamine™ 2000 was used as transfecting agent. After treatment, cells were lysed and subjected to the TRAP assay. Telomerase activity was normalized relative to a mock transfection (without RIPtide), used as negative control. A 2′-O-methyl oligonucleotide (13-mer) complementary to the template region was used as positive control (TC) IV-3 mismatch=GGUGCCAGGC (SEQ ID NO: 53) IV-5 mismatch=GGUGCCAGGC (SEQ ID NO: 53). n.d.=not determined. Error bars are s.d. of triplicates. Experiments were performed at least twice with similar results.

FIG. 9 depicts various structural components of human telomerase. FIG. 9A shows the CR4-CR5 and the pseudoknot/template domains of human telomerase. FIG. 9A discloses ‘CAAUCCCAAUC’ as SEQ ID NO: 70. FIG. 9B shows the CR4-CR5 domain, including the J5/6 loop. FIG. 9C indicates potential target sites (white) for binding the CR4-CR5 domain. FIG. 9D depicts the location of the SEQ ID NO:1 binding target site on the J5/6 loop of the CR4-CR5 domain FIG. 9D discloses ‘GCCUCCAG’ as SEQ ID NO: 1.

### DETAILED DESCRIPTION OF THE INVENTION

Inappropriate expression of telomerase is implicated in many tumor types. The RNA component of human telomerase (hTR) is necessary for the activity of the telomerase holoenzyme. Agents that bind to the RNA component of human telomerase and interfere with the role of hTR in enzyme activity or regulation can provide inhibitors of telomerase activity.

Described herein are nucleic acid agents and analogs thereof that bind to hTR and inhibit telomerase activity. In particular, nucleic acids, preferably ribonucleic acids and analogs thereof, that bind one of two different domains of the hTR, referred to herein as the CR4-CR5 domain and the pseudoknot/template domain are described. Particular sequence for these inhibitor nucleic acid molecules are provided herein, as are a variety of nucleic acid analogs of these molecules, the analogs retaining the ability to bind hTR and inhibit telomerase activity, but modified in one or more ways relative to naturally occurring nucleic acid molecules.

Also described herein are methods for inhibiting telomerase activity in a subject in need thereof. Methods are also described herein for treating cancer by administering a telomerase inhibitor as described herein. Also described herein are uses for nucleic acid agents and nucleic acid analogs thereof for the preparation of a medicament that binds to hTR and inhibit telomerase activity in a subject in need thereof.

The following descriptions provide guidance with respect to these aspects of the methods and compositions described herein.

### Telomerase RNA Structure and Relationship to Function

Human telomerase is a specialized ribonucleoprotein composed of two essential components, a reverse transcriptase protein subunit (hTERT), and an RNA component (hTR) (SEQ ID NO:71) (J. Feng, Science 269, 1236-1241 (1995); T. M. Nakamura, Science 277, 911-912 (1997)), as well as several associated proteins. It directs the synthesis of telomeric repeats (5′-TTAGGG-3′) at chromosome ends, using a short sequence within the RNA component as a template. Telomerase is considered to be an almost universal marker for human cancer, its effect on telomere length playing a crucial role in evading replicative senescence. As defined herein, “human telomerase” refers to the ribonucleoprotein complex that reverse transcribes a portion of its RNA subunit during the synthesis of G-rich DNA at the 3′ end of each chromosome in most eukaryotes, thus compensating for the inability of the normal DNA replication machinery to fully replicate chromosome termini. The human telomerase holoenzyme minimally comprises two essential components, a reverse transcriptase protein subunit (hTERT), and the “RNA component of human telomerase”, herein referred to as “hTR”. The RNA component of telomerase from diverse species differ greatly in their size and share little sequence homology, but do appear to share common secondary structures, and important common features include a template, a 5′ template boundary element, a large loop including the template and putative pseudoknot, referred to herein as the “pseudoknot/template region”, and a loop-closing helix. Human telomerase activity can be reconstituted by adding both the pseudoknot/template (nt 33-192) and the CR4/CR5 (nt 243-326) domains of the hTR (SEQ ID NO: 71) to hTERT in vitro and thus are the only hTR domains required for catalytic activity (V. M. Tesmer Mol Cell Biol. 19(9):6207-160 (1999)).

CR4-CR5 Domain: The CR4-CR5 domain (nt 243-326) of hTR (SEQ ID NO: 71) is a bona fide functional and structural domain. It can be provided in trans and activates the enzyme when provided on a separate molecule from the remainder of the RNA (V. M. Tesmer Mol Cell Biol. 19(9):6207-160 (1999); J. R. Mitchell, Mol Cell. 6(2):361-71 (2000)). Active telomerase can be functionally assembled with hTERT and two inactive domains of hTR comprising the template/pseudoknot domain and the CR4-CR5 domain (V. M. Tesmer, Mol Cell Biol. 19(9):6207-160 (1999). The “CR4-CR5 domain”, as defined herein, is one of two functional domains of hTR that are required for telomerase enzymatic activity in vitro and in vivo and is composed of nt 243-326 of hTR (SEQ ID NO: 71). Truncation studies have established that the functionally essential regions within the CR4-CR5 domain include the three-way junction and the L6.1 loop, as well as the region up to and including the J6 internal loop. While removal of the internal loop J6 abolishes activity, additional deletions further up the terminal stem-loop have no effect on hTERT binding or enzymatic activity, establishing the boundary of the functional region of CR4-CR5 (J. R. Mitchell, Mol Cell. 6(2):361-71 (2000)).

The essential structural features of the P6a/J6/P6b region can be summarized as follows. The loop region forms a stable secondary structure and the two paired regions P6a and P6b form standard A-form stems, but P6a is interrupted by a bulged cytosine. Local distortions affect the overall conformation of the entire region. The helical axes of the two paired regions are not coaxial, and the bulge introduces a strong over-twist that gives the RNA an unusual profile.

J6 loop: The J6 internal loop is common to all mammalian telomerases (J. L. Chen, Cell 100(5):503-14 (2000)). The “J6” loop, as defined herein, is a motif that is absent in birds, but it is present in fish and half of all reptiles. The “J6” loop is formed by nucleotides 246-256 and 300-323 of the hTR sequence (SEQ ID NO:71). The sequence that SEQ ID NO:1 targets is found within the J loop (nucleotides 248-255 of SEQ ID NO:71). In organisms where the J6 internal loop is present, the first C and the last U are conserved, except for chinchillas and guinea pigs, which have G substitutions at both positions. The conservation of these two nucleotides supports the unusual C/U pair seen in a structural ensemble. A purine is always present in the first position of the 3′ strand of the loop and the middle position of the 3′ strand varies, but it is never a G. The GC pair that terminates the loop and initiates the double-helical segment P6b is absolutely conserved. Furthermore, either C or U is present at the position 267 that would complete the putative triple, but never a purine. The small cavity in the J6 bulge shows promise as a drug target. Because the J6 bulge region is essential for CR4-CR5 domain RNA to interact with hTERT, a small molecule docked into this cavity could disrupt this interaction and abolish telomerase activity (T. C. Leeper, RNA, 11:394-403 (2005)). Substitutions within the J6 internal loop have varying but substantial affects upon telomerase activity in vitro (J. R. Mitchell, Mol Cell. 6(2):361-71 (2000)). Deletion of this loop completely abolishes the ability of the CR4-CR5 domain to interact with hTERT and to activate telomerase function. On the 3′-strand, substitutions from ACU to UUA only partially reduced activity; residues C266 and C267 can be substituted with AA and still retain activity.

Because individual nucleotides can be substituted without generally abolishing the domain's function, it is suggested that the key functional feature of this region is the distortion in the structure introduced by the internal loop. Consistent with this pronounced local backbone distortion is the presence of a reverse transcriptase pause at this site (M. Antal, Nucleic Acids Res. 30(4):912-20 (2002)). It is hypothesized that the over-twisting introduced by the internal loop allows the CR4-CR5 domain to fold onto itself or against the hTERT active site surface to generate the global structure required for activation of the enzymatic activity. This directional change may be the major role of the J6 internal loop. It has also been proposed that the predominant role of the J6 internal loop is structural with regard to establishment of interaction between this region of hTR and the hTERT protein.

The pseudoknot/template domain is one of two functional domains of hTR that are required for telomerase enzymatic activity in vitro and in vivo, the other domain being the CR4-CR5 domain, as described above. The “pseudoknot/template domain”, as defined herein, is a functional and structural domain of hTR (nt 33-192 of SEQ ID NO:71). The highly conserved pseudoknot/template domain of vertebrate TRs has been extensively investigated, owing to its predicted roles in telomerase functions and because mutations of this region of human TR are associated with several diseases (J. L. Chen, Proc Natl Acad Sci USA. 101(41):14683-4 (2004); C. A. Theimer, Curr Opin Struct Biol., 16(3):307-18 (2006)).

The structure of the human pseudoknot reported by the Feigon group contains helices p2b and p3 and loops j2b/3 and j2a/3 including nt 93-121 and nt 166-174, with U177 deleted for stability reasons. These represent all of the residues required for formation of the conserved H-type pseudoknot (C. A. Theimer, Mol Cell. 17(5):671-82 (2005)). The pseudoknot forms a well-ordered structure with the U-rich j2b/3 loop (U99-U106) residing in the major groove of helix p3 and the A-rich j2a/3 loop (C166-A173) located in the minor groove of helix p2b. Nucleotides U99-U101 of the j2b/3 loop form three U•A•U base triplets with the first three base pairs in helix p3, while A171 and A173 of the j2a/3 loop form two noncanonical base triplets. Each of these tertiary interactions was validated by mutational and thermodynamic studies on the stability of the pseudoknot. Importantly, telomerase activity has been correlated with the relative stability of these pseudoknot mutants (C. A. Theimer, Mol Cell. 17(5):671-82 (2005)). The structure of the p2b hairpin contains a unique series of polypyrimidine base pairs including three U•U base pairs and a water-mediated U•C base pair capped by a structured pentaloop (C. A. Theimer, Proc Natl Acad Sci USA. 100(2):449-54 (2003)). Interestingly, the dyskeratosis congenita-associated mutation GC(107-8)AG was found to stabilize the p2b hairpin and destabilize the pseudoknot conformation. Structurally, the basis for the increased stabilization is owed to a stabilizing YNMG-like tetraloop structure (C. A. Theimer, RNA. 9(12):1446-55 (2003)).

### Nucleic Acids and Analogs Useful for the Methods and Compositions Described Herein

The invention provides, in part, nucleic acids and analogs thereof that bind to hTR (SEQ ID NO: 71) for use in the inhibition of human telomerase, and methods of using and screening for such inhibitors.

As defined herein, the term “nucleic acid” refers to a polymer of nucleotides covalently linked together, e.g., at least two, at least three, at least four, at least five, at least six, at least seven, at least eight, at least nine, at least ten, or more. Preferably, the polymer comprises at least four or at least six nucleotides or analogs thereof. As will be appreciated by those skilled in the art, the depiction of a single strand also establishes the sequence of the complementary strand. Thus, a nucleic acid also provides the complementary strand of a depicted single strand. As will also be appreciated by those of skill in the art, many variants of a nucleic acid can be used for the same purpose as a given nucleic acid. Thus, a nucleic acid also encompasses substantially identical nucleic acids and complements thereof that inhibit telomerase by binding to a telomerase RNA component (SEQ ID NO:71). As will also be appreciated by those skilled in the art, a single strand provides a probe that can hybridize to a target sequence under appropriate hybridization conditions, including, for example, stringent hybridization conditions. Thus, a nucleic acid also encompasses a probe that hybridizes under appropriate hybridization conditions.

Nucleic acids can be single stranded or double stranded, or can contain portions of both double stranded and single stranded sequence. The nucleic acid can be deoxyribonucleic acid (DNA), both genomic DNA and cDNA, ribonucleic acid (RNA), or a hybrid, where the nucleic acid can contain combinations of deoxyribo- and ribo-nucleotides, and combinations of bases, including, but not limited to, uracil, adenine, thymine, cytosine, guanine, inosine, xanthine, hypoxanthine, isocytosine, isoguanine, pseudorindine, dihydrouridine, gueosine, wyosine, thiouridine, diaminopurine, isoguanosine, and diaminopyrimidine. Nucleic acids can be obtained by chemical synthesis methods or by recombinant methods.

A nucleic acid will generally contain phosphodiester bonds, although, as defined herein, a “nucleic acid analog” can be included for the purposes of the present invention that can have at least one different linkage, e.g., 2′-O-methyl all-phosphorothioate backbone, glycol nucleic acid, LNA (Locked Nucleic Acids), 2′-O-alkyl substitution, 2′-O-methyl substitution, phosphoramidate, phosphorothioate, phosphorodithioate, or O-methylphosphoroamidite linkages, phosphorodiamidate morpholino oligo backbones, and peptide nucleic acid backbones and linkages. Modifications of nucleic acids to create “nucleic acid analogs” can be done for a variety of reasons. In some embodiments, nucleic acid analogs are used to increase the stability and half-life of such molecules in physiological environments, or, in other embodiments to function as probes on a biochip. Other nucleic acid analogs include those with positive backbones; non-ionic backbones, and non-ribose backbones, including those described in U.S. Pat. Nos. 5,235,033 and 5,034,506, which are herein incorporated by reference.

As defined herein, a “locked nucleic acid” refers to a nucleotide or alternatively to a nucleic acid or analog thereof comprising such nucleotide where the ribose moiety is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the nucleic acids of the present invention whenever desired. The locked ribose conformation enhances base stacking and backbone pre-organization, and thus, significantly increases the thermal stability (melting temperature). As used herein, a “glycol nucleic acid” is a nucleic acid where the backbone is composed of repeating glycerol units linked by phosphodiester bonds. The glycerol molecule in a GNA has just three carbon atoms and still shows Watson-Crick base pairing. As defined herein, a “peptide nucleic acid” (PNA) is a nucleic acid where the backbone is composed of repeating N-(2-aminoethyl)-glycine units linked by peptide bonds. The various purine and pyrimidine bases are linked to the backbone by methylene carbonyl bonds. PNAs are depicted like peptides, with the N-terminus at the first (left) position and the C-terminus at the right. As used herein, a “threose nucleic acid” (TNA) is a nucleic acid where the backbone is composed of repeating threose units linked by phosphodiester bonds.

Nucleic acid molecules containing one or more non-naturally occurring or modified nucleotides are also included within the definition of nucleic acid analogs. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that nucleobase-modified ribonucleotides, i.e., ribonucleotides containing a non-naturally occurring nucleobase instead of a naturally occurring nucleobase, are also suitable for the purposes of the present invention and are included within the definition of a nucleic acid analog. Such nucleobase-modified ribonucloetides include but are not limited to: uridines or cytidines modified at the 5-position, e.g., 5-(2-amino)propyl uridine, 5-bromo uridine; adenosines and guanosines modified at the 8-position, e.g., 8-bromo guanosine; deaza nucleotides, e.g., 7-deaza-adenosine; O- and N-alkylated nucleotides, e.g., N6-methyl adenosine. Also included are modifications to the 2′ OH— group such as those that can be replaced by a group selected from H, OR, R, halo, SH, SR, NH2, NHR, NR2 or CN, wherein R is C-C6 alkyl, alkenyl or alkynyl and halo is F, Cl, Br or I. Mixtures of naturally occurring nucleic acids and analogs can be made; alternatively, mixtures of different nucleic acid analogs, and mixtures of naturally occurring nucleic acids and analogs can be made.

The term “derivative” as used herein refers to nucleic acid which have been chemically modified by, for example but not limited to, techniques such as methylation, acetylation, or addition of other molecules. As used herein, “variant” with reference to a polynucleotide, for example a nucleic acid or nucleic acid analog refers to a polynucleotide that can vary in primary, secondary, or tertiary structure, as compared to a reference polynucleotide respectively (e.g., as compared to a wild-type polynucleotide). A variant can also be an antisense nucleic acid strand of SEQ ID NO:1 comprising at least 1, at least 2, at least 3, at least 4, at least 5, at least 6, or at least 7 differences in any 8 contiguous nucleotides as compared to a complementary antisense nucleic acid strand of SEQ ID NO:1. A variant would also include any nucleic acid where one or more uracil nucleotides (“U”) is/are replaced with thymidine nucleotide(s) (“T”), or, as another non-limiting example, where one or more thymidine nucleotide(s) (“T”) nucleotides is/are replaced with uracil nucleotide(s) (“U”). As referred to herein, the term “differences” or “differs” in reference to a nucleic acid or nucleic acid analog sequence, refers to nucleic acid substitutions, deletions, insertions and modifications, as well as insertions of non-nucleic acid molecule, or synthetic nucleotides as disclosed herein, or nucleic acid analogs as compared to the sense strand.

The nucleic acids or nucleic acid analogs of the invention can be introduced into a cell by a variety of methods known in the art, e.g., by transfection, lipofection, electroporation, biolistics, passive uptake, lipid:nucleic acid complexes, viral vector transduction, injection, naked DNA, and the like. In some embodiments, the nucleic acids and nucleic acid analogs of the invention may be introduced using a vector or plasmid.

As used herein, the term “vector” is used interchangeably with “plasmid” and refers to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome and typically comprise entities for stable or transient expression of the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked.

As used herein, the phrase “binds to” refers to the binding of a nucleic acid or analog thereof to the RNA component of human telomerase (SEQ ID NO: 71) with a dissociation constant (Kd) of 1 μM or lower as measured using methods known in the art, such as fluorescence polarization, as described herein, or surface plasmon resonance analysis using, for example, a BIAcore, surface plasmon resonance system and BIAcore kinetic evaluation software (e.g., version 2.1). In some embodiments, the affinity or Kd (dissociation constant) for a specific binding interaction is 900 nM or lower, 800 nM or lower, 600 nM or lower, 500 nM or lower, 400 nM or lower, 300 nM or lower, or 200 nM or lower. More preferably, the affinity or Kd is 100 nM or lower, 90 nM or lower, 80 nM or lower, 70 nM or lower, 60 nM or lower, 50 nM or lower, 45 nM or lower, 40 nM or lower, 35 nM or lower, 30 nM or lower, 25 nM or lower, 20 nM or lower, 15 nM or lower, 12.5 nM or lower, 10 nM or lower, 9 nM or lower, 8 nM or lower, 7 nM or lower, 6 nM or lower, 5 nM or lower, 4 nM or lower, 3 nM or lower, 2 nM or lower, or 1 nM or lower. As used herein, the term “high affinity binding” refers to binding with a Kd of less than or equal to 100 nM.

Methods of screening for nucleic acid molecules or analogs thereof for use in the methods and compositions of the invention are also provided herein, and further illustrated, in a non-limiting manner, in the Examples. RNA-Interacting Polynucleotides (henceforth referred to herein as “RIPtides”) are recently described nucleic acid-based drugs with improved properties compared to standard unmodified DNA oligonucleotides. RIPtides have the ability to bind well-structured RNA targets with high binding affinity and specificity, with the purpose of modulating their function. The approach taken to targeting structured RNA in the present invention relates, in part, to the discovery, by means of microarrays, of short oligonucleotide sequences that can dock into pre-organized RNA sites, as determined by its intrinsic folding patterns.

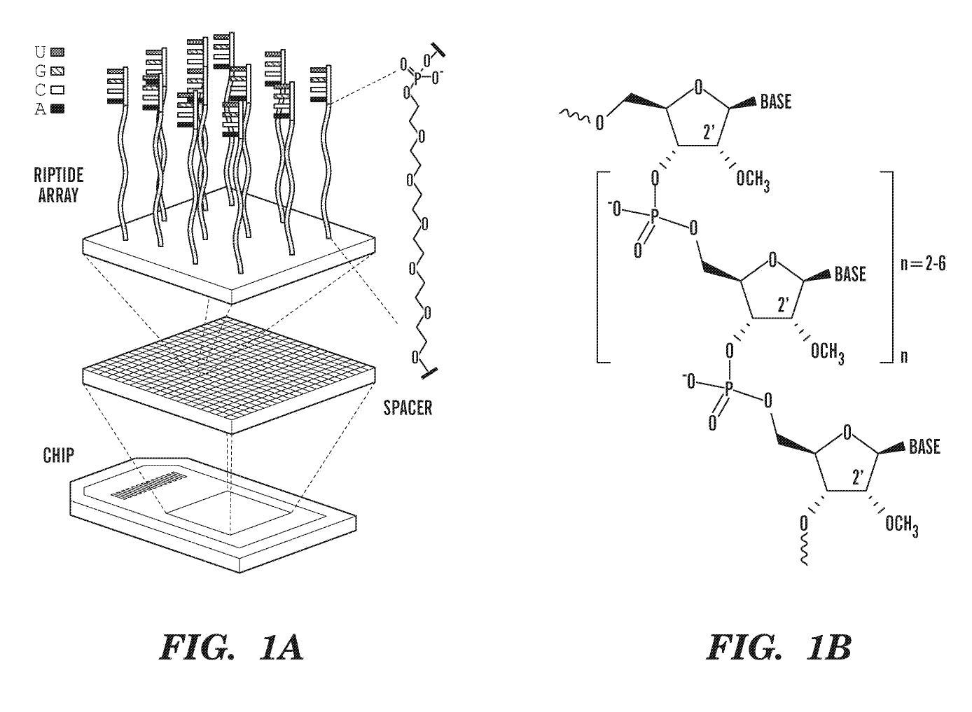

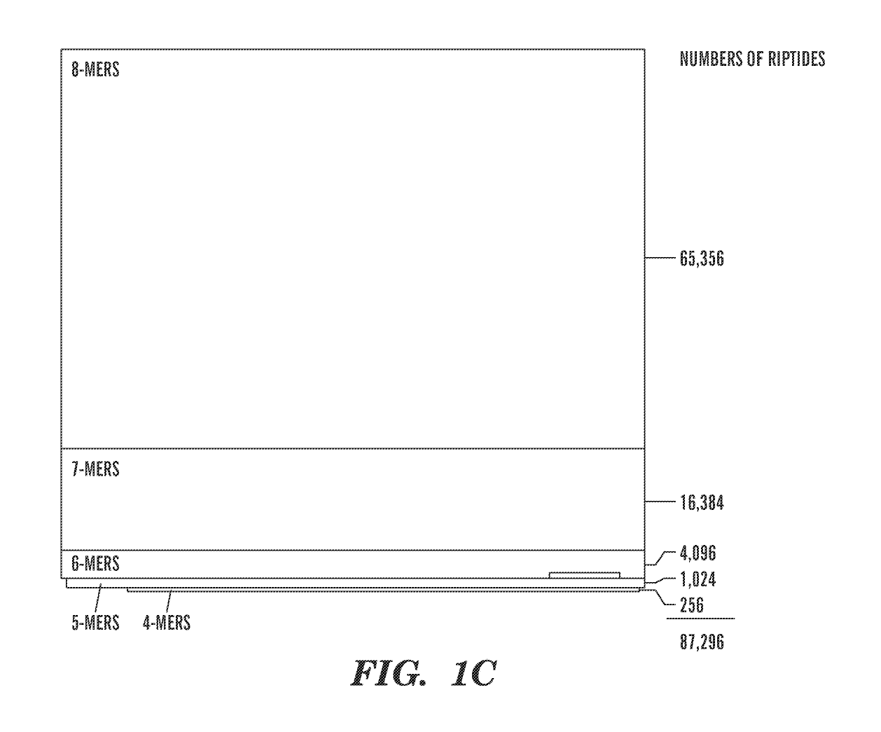

For the RIPtide discovery process, 2′-O-methyl-ribonucleotide microarrays were employed and manufactured in a custom format from Affymetrix Inc. via a photoresist-based synthesis (A. Pawloski, J. Vac. Sci. Technol. B 25, 2537-2546 (2007)). The 2′-O-Me RIPtide microarrays were generated to incorporate all possible sequences from 4-mers to 8-mers, a total of 87,296 total probes, as illustrated in FIG. 1. The microarrays described in the present work constitute the first use of high density 2′-O-Me oligonucleotide microarrays reported to date, and these were used to screen different RNA constructs of the human telomerase RNA component (hTR) (SEQ ID NO:71).

### Telomerase Inhibitors and Methods of Use

Described herein are compositions and methods for inhibiting human telomerase, by providing inhibitors that bind to the RNA component of human telomerase, including inhibitors that bind to the CR4-CR5 and the pseudoknot/template domains of the RNA component of human telomerase.

Accordingly, in one aspect, a telomerase inhibitor comprising a nucleic acid or analog thereof that binds to the CR4-CR5 domain of the RNA component of human telomerase is provided. In one embodiment, the nucleic acid binding to the CR4-CR5 domain of the RNA component of human telomerase is a ribonucleic acid. In another embodiment, the inhibitor binding to the CR4-CR5 domain of the RNA component of human telomerase is a nucleic acid analog. In another embodiment, the nucleic acid analog is a ribonucleic acid analog. Among the inhibitors that are described herein are telomerase inhibitors that bind to the J5/J6 loop of the CR4-CR5 domain of the RNA component of human telomerase.

In one embodiment, the telomerase inhibitor that binds to the CR4-CR5 domain of the RNA component of human telomerase comprises, or alternatively consists essentially of, or as a further alternative, consists of, a sequence selected from the group consisting of SEQ ID NO: 1-SEQ ID NO: 10.

| | |
|---|---|
| SEQ ID NO: 1: | 5′-GCCUCCAG-3′ |
| SEQ ID NO: 2: | 5′-GCCTCCAG-3′ |
| SEQ ID NO: 3: | 5′-GCCUCCAU-3′ |
| SEQ ID NO: 4: | 5′-GCCUCCUA-3′ |
| SEQ ID NO: 5: | 5′-GCCUCCCC-3′ |
| SEQ ID NO: 6: | 5′-GCCUCCA-3′ |
| SEQ ID NO: 7: | 5′-GCCUCC-3′ |
| SEQ ID NO: 8: | 5′-GCCUCCAA-3′ |
| SEQ ID NO: 9: | 5′-GCCCAACU-3′ |
| SEQ ID NO: 10: | 5′-GCCCAACT-3′ |

In another embodiment, the telomerase inhibitor that binds to the CR4-CR5 domain of the RNA component of human telomerase comprises the sequence of SEQ ID NO: 1 or SEQ ID NO: 2.

Other aspects of the invention provide methods of inhibiting telomerase activity. Among the methods for inhibiting telomerase activity that are described herein are methods comprising the use of nucleic acids or analogs thereof that bind to the CR4-CR5 domain of the RNA component of human telomerase.

In one method, a telomerase is contacted with a nucleic acid or nucleic acid analog thereof that binds to the CR4-CR5 domain of the RNA component of human telomerase. In certain embodiments, the nucleic acid is a ribonucleic acid. In other embodiments, the nucleic acid is a nucleic acid analog. In certain further embodiments, the nucleic acid is a ribonucleic acid analog. Among the inhibitors described herein for contacting a telomerase are telomerase inhibitors that bind to the J5/J6 loop of the CR4-CR5 domain of the RNA component of human telomerase.

In one embodiment, the telomerase inhibitor that binds to the CR4-CR5 domain of the RNA component of human telomerase comprises, or alternatively consists essentially of, or as a further alternative, consists of, a sequence selected from the group consisting of SEQ ID NO: 1-SEQ ID NO: 10. In another embodiment, the telomerase inhibitor that binds to the CR4-CR5 domain of the RNA component of human telomerase comprises, or alternatively consists essentially of, or as a further alternative, consists of, the sequence of SEQ ID NO: 1 or SEQ ID NO: 2.

In connection with contacting a telomerase with a nucleic acid or analog thereof that binds to the CR4-CR5 domain of the RNA component of human telomerase, “inhibiting telomerase activity” or “inhibition of telomerase activity” indicates that the telomerase activity is at least 5% lower in a telomerase treated with a nucleic acid or nucleic acid analog thereof that binds to the CR4-CR5 domain of the RNA component of human telomerase, than comparable, control telomerases, wherein no nucleic acid or nucleic acid analog thereof binding to the CR4-CR5 domain of the RNA component of human telomerase, is present. The telomerase activity can be measured using any assay or method known to one of skill in the art, including but not limited to, for example, such as the TRAP activity assays described herein. It is preferred that the telomerase activity in telomerases treated with a nucleic acid or analog thereof binding to the CR4-CR5 domain of the RNA component of human telomerase is at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, at least 95% lower, at least 98%, at least 99%, to include 100%, i.e., zero detectable activity relative to a control treated telomerase.

In another method, a cell is contacted with a nucleic acid or analog thereof that binds to the CR4-CR5 domain of the RNA component of human telomerase. In certain embodiments the nucleic acid is a ribonucleic acid. In other embodiments, the nucleic acid is a nucleic acid analog. In certain further embodiments, the nucleic acid is a ribonucleic acid analog. Included among the inhibitors described herein for contacting a cell to inhibit telomerase activity are telomerase inhibitors that bind to the J5/J6 loop of the CR4-CR5 domain of the RNA component of human telomerase.

In one embodiment, the telomerase inhibitor contacting the cell comprises a sequence selected from the group consisting of SEQ ID NO: 1-SEQ ID NO: 10. In another embodiment, the telomerase inhibitor contacting the cell and binds to the CR4-CR5 domain of the RNA component of human telomerase includes the sequence of SEQ ID NO: 1 or SEQ ID NO: 2.

In connection with contacting a cell with a nucleic acid or analog thereof that binds to the CR4-CR5 domain of the RNA component of human telomerase, “inhibiting telomerase activity” or “inhibition of telomerase activity” indicates that the telomerase activity is at least 5% lower in a cell treated with a nucleic acid or analog thereof that binds to the CR4-CR5 domain of the RNA component of human telomerase, than a comparable, control cell, where no nucleic acid or analog thereof binding to the CR4-CR5 domain of the RNA component of human telomerase, is present. It is preferred that the telomerase activity in a cell treated with a nucleic acid or analog thereof binding to the CR4-CR5 domain of the RNA component of human telomerase is at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, at least 95% lower, at least 98%, at least 99%, to include 100%, i.e., zero detectable activity, relative to a control treated cell.

The phrases “control treated telomerase” or “control treated cell”, are used herein to describe a telomerase or cell that has been treated with identical media, viral induction, nucleic acid sequences, temperature, confluency, flask size, pH, etc., with the exception of the addition of a nucleic acid or analog thereof that binds to the CR4-CR5 domain of the RNA component of human telomerase.

Also described herein are methods and compositions for inhibiting human telomerase, by providing inhibitors that bind to the pseudoknot/template domain of the RNA component of human telomerase.

Accordingly, in one aspect, a telomerase inhibitor comprising a ribonucleic acid molecule or analog thereof that binds to the pseudoknot/template domain of the RNA component of human telomerase is provided, where the ribonucleic acid molecule or analog thereof comprises, or alternatively consists essentially of, or as a further alternative, consists of, a binding sequence selected from the group consisting of SEQ ID NO: 11-SEQ. ID NO: 45. In one embodiment, the telomerase inhibitor comprises, or alternatively consists essentially of, or as a further alternative, consists of, a binding sequence selected from the group consisting of SEQ ID NO: 19-SEQ ID NO: 24; SEQ ID NO: 39; SEQ ID NO: 44; and SEQ ID NO: 45. In another embodiment, the telomerase inhibitor binding sequence comprises, or alternatively consists essentially of, or as a further alternative, consists of, the sequence of SEQ. ID NO: 20.

SEQ ID NO: 11: GUCAGCGA (II-2)
SEQ ID NO: 12: AGCGAGAA (II-3)
SEQ ID NO: 13: GUCAGCGAGAAA (II-5)
SEQ ID NO: 14: GGAGCA (III-1)
SEQ ID NO: 15: GGAGCAAA (III-2)
SEQ ID NO: 16: GGAGCAAAAGCA (III-3)
SEQ ID NO: 17: GGAGCAAAAG (III-4)
SEQ ID NO: 18: GGGAGCAAAA (III-5)
SEQ ID NO: 19: GAACGGUG (IV-2)
SEQ ID NO: 20: GGUGGAAGGC (IV-3)
SEQ ID NO: 21: GAACGGUGGAAGGC (IV-4)
SEQ ID NO: 22: ACGGUGGAAGGC (IV-6)
SEQ ID NO: 23: GGUGGAAG (IV-7)
SEQ ID NO: 24: GGUGGAAGG (IV-8)
SEQ ID NO: 25: AGGGUUAG (V-2)
SEQ ID NO: 26: AGUUAGG (V-3)
SEQ ID NO: 27: GUCAGCGAGAAAA
SEQ ID NO: 28: CAGCGAGA
SEQ ID NO: 29: GACAGCGC
SEQ ID NO: 30: CAGCGAGG
SEQ ID NO: 31: ACAGCGAG
SEQ ID NO: 32: AACAGCGC
SEQ ID NO: 33: CAGCGAG
SEQ ID NO: 34: UCAGCGAG
SEQ ID NO: 35: ACAGCGCA
SEQ ID NO: 36: AGUCAGCG
SEQ ID NO: 37: AACAGCGC
SEQ ID NO: 38: ACAGCGC
SEQ ID NO: 39: GAAGGCG
SEQ ID NO: 40: GGGAGCAAAA
SEQ ID NO: 41: GCGGGAGCAAAA
SEQ ID NO: 42: GAAGGCG
SEQ ID NO: 43: GGUGGAAGGC
SEQ ID NO: 44: CGGUGGAAGG
SEQ ID NO: 45: GAACGGUGGAA

Other aspects of the invention provide methods of inhibiting telomerase activity comprising the use of nucleic acids or analogs thereof that bind to the pseudoknot/template domain of the RNA component of human telomerase. In one such method, a cell is contacted with a ribonucleic acid molecule or analog thereof that binds to the pseudoknot/template domain of the RNA component of human telomerase, where the ribonucleic acid molecule or analog thereof comprises, or alternatively consists essentially of, or as a further alternative, consists of, a binding sequence selected from the group consisting of SEQ ID NO: 11-SEQ. ID NO: 45. In one embodiment, the ribonucleic acid molecule or ribonucleic acid analog thereof comprises, or alternatively consists essentially of, or as a further alternative, consists of, a binding sequence selected from the group consisting of SEQ ID NO: 19-SEQ ID NO: 24; SEQ ID NO: 39; SEQ ID NO: 44; and SEQ ID NO: 45. In another embodiment, the telomerase binding sequence comprises, or alternatively consists essentially of, or as a further alternative, consists of, the sequence of SEQ. ID NO: 20.

The term “cell”, as used herein, refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, transgenic animal domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, stromal, muscle, cardiovascular, hepatic, pulmonary, renal, gastrointestinal stem cells, etc. Yeast cells may also be used as cells in this invention. Cells also refer not to a particular subject cell but to the progeny or potential progeny of such a cell because of certain modifications or environmental influences, for example differentiation, such that the progeny may not, in fact be identical to the parent cell, but are still included in the scope of the invention. The cells used in the invention can also be cultured cells, e.g. in vitro or ex vivo. For example, cells cultured in vitro in a culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art. Cells used in the invention can be present in a subject, e.g. in vivo. For the invention on use on in vivo cells, the cell is preferably found in a subject and display characteristics of the disease, disorder, or malignancy pathology.

As used herein the term “sample” or “biological sample” mean any sample, including but not limited to cells, organisms, lysed cells, cellular extracts, nuclear extracts, or components of cells or organisms, extracellular fluid, and media in which cells are cultured.

### Therapeutic Applications of Telomerase Inhibitors

In certain aspects, the invention provides methods and compositions for the treatment of various disorders. The methods involve administering to a subject in need thereof a therapeutically effective amount of one or more of the telomerase inhibitors described herein.

Among the methods for treatment described herein for inhibiting telomerase activity in a subject in need thereof are methods comprising the use of nucleic acids or analogs thereof that bind to the CR4-CR5 domain of the RNA component of human telomerase.

Accordingly, one aspect provides a method of treating a proliferative disorder in a subject in need thereof, comprising administering to the subject an effective amount of a telomerase inhibitor comprising a nucleic acid or analog thereof that binds to the CR4-CR5 domain of the RNA component of human telomerase.

In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. Among the inhibitors described herein for treating a subject with a proliferative disorder in need thereof, are telomerase inhibitors that bind to the J5/J6 loop of the CR4-CR5 domain of the RNA component of human telomerase.

In one embodiment, the telomerase inhibitor that binds to the CR4-CR5 domain of the RNA component of human telomerase comprises, or alternatively consists essentially of, or as a further alternative, consists of, a sequence selected from the group consisting of SEQ ID NO: 1-SEQ. ID NO: 10. In a preferred embodiment, the telomerase inhibitor comprises, or alternatively consists essentially of, or as a further alternative, consists of, the sequence SEQ ID NO: 1 or SEQ ID NO: 2. In one embodiment, the proliferative disorder being treated in the subject is a cancer.

Another aspect provides the use of a telomerase inhibitor comprising an effective amount of a nucleic acid or analog thereof that binds to the CR4-CR5 domain of the RNA component of human telomerase in the manufacture of a medicament for treating a proliferative disorder in a subject in need thereof.

In one embodiment, the nucleic acid binding to the CR4-CR5 domain of the RNA component of human telomerase is a ribonucleic acid. In another embodiment, the inhibitor is a nucleic acid analog. In another embodiment, the nucleic acid analog is a ribonucleic acid analog. Among the inhibitors described herein for treating a subject with a proliferative disorder in need thereof, are telomerase inhibitors that bind to the J5/J6 loop of the CR4-CR5 domain of the RNA component of human telomerase.

In one embodiment, the telomerase inhibitor that binds to the CR4-CR5 domain of the RNA component of human telomerase comprises, or alternatively consists essentially of, or as a further alternative, consists of, a sequence selected from the group consisting of SEQ ID NO: 1-SEQ. ID NO: 10. In a preferred embodiment, the telomerase inhibitor comprises, or alternatively consists essentially of, or as a further alternative, consists of, the sequence SEQ ID NO: 1 or SEQ ID NO: 2. In one embodiment, the proliferative disorder being treated in the subject is a cancer.

Described herein are also methods for treatment for inhibiting telomerase activity in a subject in need thereof comprising the use of nucleic acids or analogs thereof that bind to the pseudoknot/template domain of the RNA component of human telomerase.

Accordingly, one aspect provides a method of treating a proliferative disorder in a subject in need thereof, comprising administering to the subject an effective amount of a telomerase inhibitor, the telomerase inhibitor comprising a ribonucleic acid molecule or analog thereof that binds to the pseudoknot/template domain of the RNA component of human telomerase, and where said ribonucleic acid molecule or analog thereof comprises, or alternatively consists essentially of, or as a further alternative, consists of, a binding sequence selected from the group consisting of SEQ ID NO: 11-SEQ. ID NO: 45. In one embodiment, the binding sequence of the ribonucleic acid molecule or analog thereof comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45. In another embodiment, the telomerase binding sequence comprises, or alternatively consists essentially of, or as a further alternative, consists of, the sequence of SEQ. ID NO: 20. In one embodiment, the proliferative disorder is a cancer.

Another aspect of the invention provides the use of an effective amount of a telomerase inhibitor, comprising a ribonucleic acid molecule or analog thereof that binds to the pseudoknot/template domain of the RNA component of human telomerase, in the manufacture of a medicament for treating a proliferative disorder in a subject in need thereof. In one embodiment ribonucleic acid molecule or analog thereof comprises, or alternatively consists essentially of, or as a further alternative, consists of, a binding sequence selected from the group consisting of SEQ ID NO: 11-SEQ. ID NO: 45. In one embodiment, the binding sequence of the ribonucleic acid molecule or analog thereof comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45. In another embodiment, the telomerase binding sequence comprises, or alternatively consists essentially of, or as a further alternative, consists of, the sequence of SEQ. ID NO: 20. In one embodiment, the proliferative disorder is a cancer.

With reference to the methods for treatment of a subject with a proliferative disorder by administering to the subject an effective amount of a telomerase inhibitor comprising a nucleic acid or analog thereof, as disclosed herein, the terms “treat” or “treatment” or “treating” refers to both therapeutic treatment and prophylactic or preventative measures, wherein the administration in a clinically appropriate manner prevents or slows the development of the disorder, such as slows down the development of a tumor, or the spread of cancer, or reduces at least one effect or symptom of a condition, disease, or disorder associated with the inappropriate proliferation of a cell mass, for example cancer.

Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of the disorder, stabilized (i.e., not worsening) state of the disorder, delay or slowing of the disorder's progression, amelioration or palliation of the state of the disorder, and remission (whether partial or total), whether detectable or undetectable. “Treatment” can also mean prolonging survival as compared to expected survival if not receiving treatment. Those in need of treatment include those already diagnosed with cancer, as well as those likely to develop secondary tumors due to metastasis.

The terms “effective” and “effectiveness”, as used herein, includes both pharmacological effectiveness and physiological safety. Pharmacological effectiveness refers to the ability of the treatment to result in a desired biological effect in the subject. Hence, in connection with administering to a subject an effective amount of a telomerase inhibitor, an “effective amount” of a telomerase inhibitor indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of cancer being treated in the subject in need. Physiological safety refers to the level of toxicity, or other adverse physiological effects at the cellular, organ and/or organism level (often referred to as side-effects) resulting from administration of the treatment. “Less effective” means that the treatment results in a therapeutically significant lower level of pharmacological effectiveness and/or a therapeutically greater level of adverse physiological effects.

The term “therapeutically effective amount” refers also to the amount that is safe and sufficient to prevent or delay the development and further growth of a tumor or the spread of metastases in a subject with a cancer. The amount can thus cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastases. The effective amount for the treatment of cancer depends on the tumor to be treated, the severity of the tumor, the drug resistance level of the tumor, the species being treated, the age and general condition of the subject, the mode of administration and so forth. Thus, it is not possible to specify a single, exact “effective amount”. However, for any given case, an appropriate “effective amount” can be determined by one of ordinary skill in the art using only routine experimentation.

A therapeutically effective amount of the agents, factors, or inhibitors described herein, or functional derivatives thereof, for inhibiting telomerase activity can vary according to factors such as disease state, age, sex, and weight of the subject, and the ability of the therapeutic compound to elicit a desired response in the individual or subject. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. For example, efficacy can be assessed in animal models of cancer and tumor, i.e., treatment of a rodent with a cancer, and any treatment or administration of the compositions or formulations that leads to a decrease of at least one symptom of the cancer, for example a reduction in the size of the tumor or a slowing or cessation of the rate of growth of the tumor indicates effective treatment. In embodiments where inhibitors of telomerase activity are used for the treatment of cancer, the efficacy can be judged using an experimental animal model of cancer, e.g., wild-type mice or rats, or transplantation of tumor cells.

When using an experimental animal model, efficacy of treatment is evidenced when a reduction in a symptom of the cancer, for example a reduction in the size of the tumor or a slowing or cessation of the rate of growth of the tumor occurs earlier in treated, versus untreated animals. By “earlier” is meant that a decrease, for example in the size of the tumor, occurs at least 5% earlier, but preferably more, e.g., one day earlier, two days earlier, 3 days earlier, or more. As used herein, the term “treating” when used in reference to a cancer treatment is used to refer to the reduction of a symptom and/or a biochemical marker of cancer, for example a reduction in at least symptom or one biochemical marker of cancer by at least about 10% would be considered an effective treatment. In some embodiments, a treatment would be considered if there was a reduction of at least about 20%, at least about 30%, at least about 40%, at least about 50%, at least about 60%, at least about 70%, at least about 80%, at least about 90%, or at least 100%, i.e., there were no longer any sign of the symptom or biochemical marker. Examples of such biochemical markers of cancer include CD44, telomerase, TGF-α, TGF-β, erbB-2, erbB-3, MUC1, MUC2, CK20, PSA, CA125 and FOBT. A reduction in the rate of proliferation of the cancer cells by at least about 10% would also be considered effective treatment by the methods as disclosed herein. As alternative examples, a reduction in a symptom of cancer, for example, a slowing of the rate of growth of the cancer by at least about 10% or a cessation of the increase in tumor size, or a reduction in the size of a tumor by at least about 10% or a reduction in the tumor spread (i.e. tumor metastasis) by at least about 10% would also be considered as affective treatments by the methods as disclosed herein. In some embodiments, it is preferred, but not required that the therapeutic agent actually kill the tumor.

A “cancer” refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a patient, or may be a non-tumorigenic cancer cell, such as a leukemia cell. In some circumstances, cancer cells will be in the form of a tumor; such cells may exist locally, or circulate in the blood stream as independent cells, for example, leukemic cells. Examples of cancer include, but are not limited to, breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

The terms “subject” and “individual” are used interchangeably herein, and refer to an animal, for example, a human from whom cells can be obtained, as described herein. For treatment of conditions or disease states which are specific for a specific animal such as a human subject, the term subject refers to that specific animal. The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like are also encompassed in the term subject.

In connection with administering an effective amount of a telomerase inhibitor to a subject in need thereof, the route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The compositions and inhibitors of the invention can be administered parenterally by injection or by gradual infusion over time and can be delivered by peristaltic means. Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics. For topical administration, the pharmaceutical composition (i.e., inhibitor of telomerase activity) is formulated into ointments, salves, gels, or creams, as is generally known in the art. The therapeutic compositions of this invention can be administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

In general, any method of delivering a nucleic acid molecule can be adapted for use with the nucleic acid or analog thereof telomerase inhibitors of the present invention (see e.g., Akhtar S, and Julian R L. (1992) Trends Cell. Biol. 2(5):139-144; WO94/02595, which are incorporated herein by reference in their entirety). Methods of delivering a telomerase inhibitor to the target cells, e.g., a cancer cell or other desired target cells, for uptake can include injection of a composition containing a telomerase inhibitor, e.g., a nucleic acid or nucleic acid analog specific for the CR4/CR5 or pseudoknot/template domain of human telomerase, or directly contacting the cell, e.g., a lymphocyte, with a composition comprising a telomerase inhibitor, e.g., a nucleic acid or nucleic acid analog specific for the CR4/CR5 or pseudoknot/template domain of human telomerase.

Important factors to consider in order to successfully deliver a nucleic acid or nucleic acid analog telomerase inhibitor in vivo, include, for example: (1) biological stability of the nucleic acid or nucleic acid analog, (2) preventing non-specific effects, and (3) accumulation of the nucleic acid or nucleic acid analog molecule in the target tissue. The non-specific effects of a telomerase inhibitor can be minimized by local administration by e.g., direct injection into a tumor, cell, target tissue, or topically. Local administration of a telomerase inhibitor molecule to a treatment site limits the exposure of the e.g., a nucleic acid or nucleic acid analog specific for the CR4/CR5 or pseudoknot/template domain of human telomerase, to systemic tissues and permits a lower dose of the nucleic acid or nucleic acid analog molecule to be administered (for example, Tolentino, M J., et al (2004) Retina 24:132-138; Reich, S J., et al (2003) Mol. Vis. 9:210-216).

For administering a nucleic acid or analog telomerase inhibitor systemically for the treatment of a disease, a nucleic acid or nucleic acid analog can be modified, or alternatively, delivered using a drug delivery system that minimize exposure to degrading factors and thus act to prevent the rapid degradation of the nucleic acid analog thereof telomerase inhibitor by, for example, endo- and exo-nucleases in vivo. Modification of the nucleic acid or analog thereof telomerase inhibitor or the pharmaceutical carrier can also permit targeting to the target tissue and avoid undesirable off-target effects.

Nucleic acid or nucleic acid analog telomerase inhibitors can be modified by chemical conjugation to lipophilic groups such as cholesterol to enhance cellular uptake and prevent degradation (Soutschek, J., et al (2004) Nature 432:173-178), and can be conjugated to an aptamer to inhibit tumor growth and mediate tumor regression (McNamara, JO., et al (2006) Nat. Biotechnol. 24:1005-1015).

In other embodiments, the nucleic acid or analog thereof telomerase inhibitors can be delivered using drug delivery systems such as e.g., a nanoparticle, a dendrimer, a polymer, or a liposomal, or cationic delivery system. Positively charged cationic delivery systems facilitate binding (nucleic acids are negatively charged) and also enhance interactions at the negatively charged cell membrane to permit efficient uptake by the cell. Cationic lipids, dendrimers, or polymers can either be bound to a nucleic acid or nucleic acid analog telomerase inhibitor, or induced to form a vesicle or micelle (see e.g., Kim S H., et al (2008) Journal of Controlled Release 129(2):107-116) that encases the nucleic acid or nucleic acid analog. The formation of vesicles or micelles further prevents degradation when administered systemically. Methods for making and administering cationic-nucleic acid or nucleic acid analog complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205).

Some non-limiting examples of drug delivery systems useful for systemic administration of a nucleic acid or nucleic acid analog telomerase inhibitor include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, a nucleic acid or nucleic acid analog telomerase inhibitor forms a complex with cyclodextrin for systemic administration (U.S. Pat. No. 7,427,605).

In other embodiments, a nucleic acid or nucleic acid analog telomerase inhibitor, e.g., a nucleic acid or analog specific for the CR4/CR5 or pseudoknot/template domain of human telomerase, may be injected directly into any blood vessel, such as vein, artery, venule or arteriole, via, e.g., hydrodynamic injection or catheterization. Administration may be by a single injection or by two or more injections. The nucleic acid or nucleic acid analog telomerase inhibitor is delivered in a pharmaceutically acceptable carrier. One or more nucleic acid or nucleic acid analog telomerase inhibitors may be used simultaneously. In one embodiment, specific cells are targeted, limiting potential side effects caused by non-specific targeting of the nucleic acid or nucleic acid analog telomerase inhibitor. The method can use, for example, a complex or a fusion molecule comprising a cell targeting moiety and a nucleic acid or nucleic acid analog binding moiety that is used to deliver the nucleic acid or nucleic acid analog effectively into cells, for example, an antibody-protamine fusion protein. Plasmid- or viral-mediated delivery mechanism can also be employed to deliver the nucleic acid or nucleic acid analog to cells in vitro and in vivo (Xia, H. et al. (2002) Nat Biotechnol 20(10):1006); Rubinson, D. A., et al. ((2003) Nat. Genet. 33:401-406; Stewart, S. A., et al. ((2003) RNA 9:493-501).

### Pharmaceutical Compositions Comprising Telomerase Inhibitors

Described herein are also pharmaceutical compositions comprising nucleic acids or analogs thereof for inhibiting telomerase activity and modes of administration therein.

Accordingly, in one aspect a therapeutic composition is provided, comprising a telomerase inhibitor and a pharmaceutically acceptable carrier, where the telomerase inhibitor comprises a nucleic acid or analog thereof that binds to the CR4-CR5 domain of the RNA component of human telomerase.

In one embodiment, the nucleic acid binding to the CR4-CR5 domain of the RNA component of human telomerase is a ribonucleic acid. In another embodiment, the nucleic acid is a nucleic acid analog. In another embodiment, the nucleic acid analog is a ribonucleic acid analog. Among the inhibitors described herein, are inhibitors that bind to the J5/J6 loop of the CR4-CR5 domain of the RNA component of human telomerase. In one embodiment, the telomerase inhibitor that binds to the CR4-CR5 domain of the RNA component of human telomerase comprises, or alternatively consists essentially of, or as a further alternative, consists of, a sequence selected from the group consisting of SEQ ID NO: 1-SEQ. ID NO: 10. In a preferred embodiment, the telomerase inhibitor comprises, or alternatively consists essentially of, or as a further alternative, consists of, a sequence of SEQ ID NO: 1 or SEQ ID NO: 2.

Accordingly, in another aspect, the invention provides a therapeutic composition comprising a telomerase inhibitor and a pharmaceutically acceptable carrier, where the telomerase inhibitor comprises a nucleic acid or analog thereof that binds to the pseudoknot/template domain of the RNA component of human telomerase. In one embodiment, the nucleic acid molecule, e.g., ribonucleic acid molecule, or analog thereof comprises, or alternatively consists essentially of, or as a further alternative, consists of, a binding sequence selected from the group consisting of SEQ ID NO: 11-SEQ. ID NO: 45. In another embodiment, the binding sequence of the ribonucleic acid molecule or analog thereof comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45. In another embodiment, the telomerase binding sequence comprises, or alternatively consists essentially of, or as a further alternative, consists of, the sequence of SEQ. ID NO: 20.

Any formulation or drug delivery system containing the active ingredients required for inhibition of telomerase activity, suitable for the intended use, as are generally known to those of skill in the art, can be used. As used herein, the terms “pharmaceutically acceptable”, “physiologically tolerable” and grammatical variations thereof, as they refer to compositions, carriers, diluents and reagents, are used interchangeably and refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier”, as used herein, means a pharmaceutically acceptable material, composition or vehicle, such as a liquid or solid filler, diluent, excipient, solvent or encapsulating material, combined with a nucleic acid or analog thereof as described herein for in vivo delivery of the nucleic acid or analog thereof.

In addition to being “pharmaceutically acceptable” as that term is defined herein, each carrier must also be “acceptable” in the sense of being compatible with the other ingredients of the formulation. A pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery compositions of the invention are formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, see, for example, University of the Sciences in Philadelphia (2005) Remington: The Science and Practice of Pharmacy with Facts and Comparisons, 21st Ed.

The present invention is further explained in detail by the following examples, but the scope of the invention should not be limited thereto. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the Detailed Description, the drawings, and the claims.

### EXAMPLES

During the last few years, the field of cancer drug discovery has experienced notable advances in terms of understanding the crucial requirements in the search for selective and efficient drugs as well as the rationale used for the selection of molecular targets (S. L. Mooberry, Drug Discovery Handbook, 1343-1368 (2005)). Small-molecule based ligands that can fit into well-defined hydrophobic pockets of proteins are still regarded as the classical drug options and proteins the most prevalent therapeutic targets within the “druggable” genome (A. L. Hopkins, Nat. Rev. Drug Discovery 1, 727-730 (2002)).

Notwithstanding that nearly all therapeutic agents developed to date target proteins, it is now widely recognized that only a minority of proteins are capable of being targeted (A. L. Hopkins, Nat. Rev. Drug Discovery 1, 727-730 (2002)). The realization that most proteins are considered “undruggable” has fueled efforts to develop the therapeutic potential of alternative classes of macromolecular targets, with RNA being the object of most intensive investigation (Lagoja, I. M. and Herdewijn, P. Expert Opin. Drug Discov. 2, 889-903 (2007). Thomas, J. R. and Hergenrother, P. J. Chem. Rev. 108, 1171-1224 (2008)).

In particular, RNA has been relegated for many years as a mere carrier of genetic information, despite its many roles in diverse cellular processes (ribozymes, riboswitches, miRNAs). The intrinsic possibilities for therapeutic intervention, that include but are not limited to the possibility of controlling gene expression by using traditional (antisense) and recent (RNAi) approaches, have resulted in a growing interest in understanding RNA structure and function. Although extremely challenging and elusive, efforts aimed at targeting RNA with small molecules hold great promise, and the inherently flexible and complex structure of RNA could in principle be used as a basis for rational design of novel strategies aimed at disrupting its function (J. R. Thomas, Chem. Rev. 108, 1171-1224 (2008)). This could be especially relevant not only to targeting messenger RNAs, but to targeting other well-structured, non-coding RNAs that play essential roles in a cellular context.

Though examples are known of small molecules that target RNA potently and specifically (Thomas, J. R. and Hergenrother, P. J. Chem. Rev. 108, 1171-1224 (2008); Hermann T., Cell. Mol. Life. Sci. 64, 1841-1852 (2007); Welch, E. M, et al. Nature 447, 87-91 (2007)), such cases are rare, hence most efforts to target RNA have taken advantage of the fact that naturally occurring nucleic acids target each other quite efficiently through nucleobase-pairing. Antisense oligonucleotides, small interfering RNAs, ribozymes, DNAzymes and nucleic acid-targeting aptamers all engage a contiguous stretch of the target RNA through sequence-complementary nucleobase-pairing interactions, predominantly of the Watson-Crick type (Lagoja, I. M. and Herdewijn, P. Expert Opin. Drug Discov. 2, 889-903 (2007)). By its very nature, this mode of engagement requires that the target sequence be minimally tied up in competing base-pairing interactions. This restriction presents one of the greatest challenges in the practice of RNA targeting, as most RNA sequences participate extensively in self-pairing, and both the structural nature of this intrastrand pairing and the energetic cost of competing with it cannot be predicted with precision.

The novel work described herein provides unbiased identification of readily targetable stretches in complex RNA molecules. The present studies also describe a screen that, by design, enables the discovery of non-canonical binders. The recent explosion in the availability of high-resolution structures of folded RNA molecules has revealed tremendous diversity in the modes by which RNA interacts autologously. Hoogsteen pairing, base triples and quadruples, structured internal and hairpin loops, pseudoknot structures, bulges, and junctions all augment canonical pairing (Leontis, N. B., et al., Curr. Opin. Struct. Biol. 16, 279-287 (2006); Hendrix, D. K., et al., Q. Rev. Biophys. 38, 221-243 (2005)). It is recognized herein that because RNA can employ such a wide variety of interactions to stabilize intramolecular association (i.e., folding), then it stands to reason that agents that target RNA intermolecularly might also employ such non-canonical interactions. Whereas there exist highly predictable pairing rules for binders that employ canonical pairing to a contiguous stretch in the RNA target, such rules do not exist for binders that employ less canonical recognition modes, necessitating the use of oligonucleotide library screening to discover the latter.

RNA-Interacting Polynucleotides (designated as “RIPtides”) are candidate nucleic acid-based drugs with improved properties compared to standard unmodified DNA oligonucleotides, and are endowed with the ability to bind well-structured RNA targets with high binding affinity and specificity, with the purpose of modulating their function. Short oligonucleotides have been previously reported to possess relevant properties in the RNA targeting arena. ODMiR (Oligonucleotide Directed Misfolding of RNA), for example, has proven to be an effective method for the inhibition of group I introns and E. Coli RNase P (J. L. Childs, Proc. Natl. Acad. Sci. USA 99, 11091-11096 (2002); J. L. Childs, RNA 9, 1437-1445 (2003)).

Described herein is a novel approach toward the discovery of RNA-interacting polynucleotides (RIPtides) that can bind to folded RNA targets is described. This method is completely unbiased with regard to pairing mode but is biased toward targetable sequences. Briefly, an N-mer microarray presenting all possible nucleic acid sequences of length N=4-8, and bearing the nucleobases A, C, G and U, enabled efficient, simultaneous screening for RIPtide binders to RNA targets under reasonably physiologic conditions. Such short sequences work within practical constraints on the number of sequences presented on a single microarray, but just as importantly, such polynucleotide sequences can exhibit enhanced cell-permeability relative to the more conventional oligonucleotide long-mers (Loke, S. L., et al., Proc. Natl. Acad. Sci. USA 86, 3474-3478 (1989); Chen, Z., et al., J. Med. Chem. 45, 5423-5425 (2002)), and that relatively short nucleic acid sequences can bind tightly and specifically to RNA targets (Childs, J. L. et al., Proc. Natl. Acad. Sci. USA 99, 11091-11096 (2002); Childs, J. L., et al., RNA 9, 1437-1445 (2003)). To enhance the binding affinity and stability of the polynucleotides, 2′-O-methylated monomer building blocks were employed (Freier, S. M. and Altmann, K. H., Nucleic Acids Res. 25, 4429-4443 (1997)). The use of these analogs in microarray fabrication was made possible through a recently developed procedure that employs photochemical production of an acid to effect deprotection of the 5′-hydroxyl group to effect sector-specific polynucleotide chain extension (Pawloski, A. et al., J. Vac. Sci. Technol. B 25, 2537-2546 (2007); McGall, G. et al., Proc. Natl. Acad. Sci. USA 93, 13555-13560 (1996)).

The approach to targeting structured RNA described herein involves the discovery, by means of microarrays, of short oligonucleotide sequences that can dock into pre-organized RNA sites, as determined by its intrinsic folding patterns. For the first RIPtide discovery process, 2′-O-methyl-ribonucleotide microarrays, manufactured in a custom format from Affymetrix Inc. via photoresist-based synthesis, were employed (A. Pawloski, J. Vac. Sci. Technol. B 25, 2537-2546 (2007)). The 2′-O-Me RIPtide microarrays were generated to incorporate all possible sequences from 4-mers to 8-mers, a total of 87,296 total probes, as illustrated in FIG. 1C. To our knowledge, the microarrays described in this work constitute the first case of high density 2′-O-Me oligonucleotide microarrays reported to date.

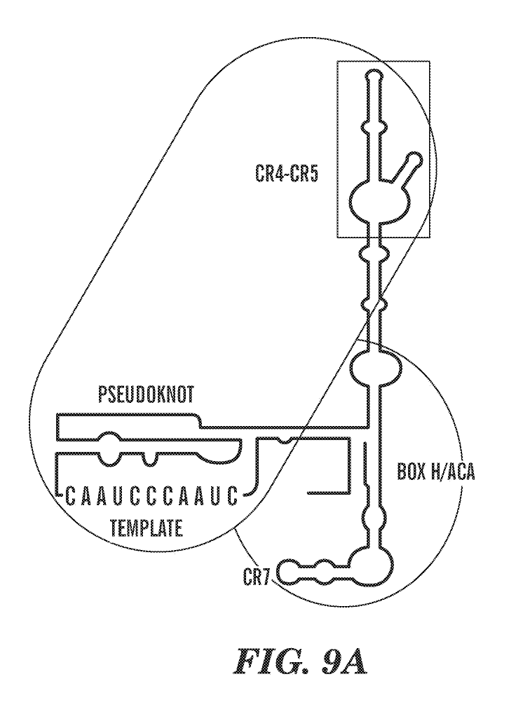

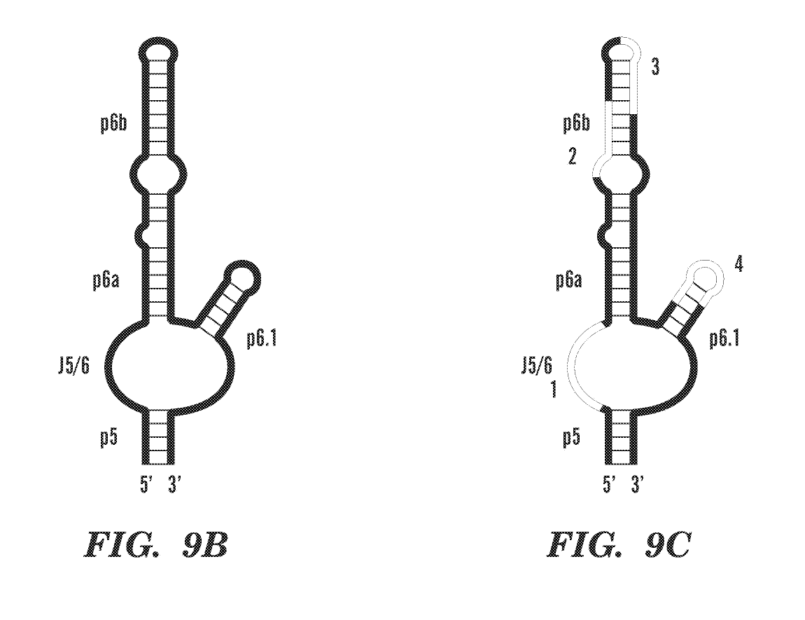

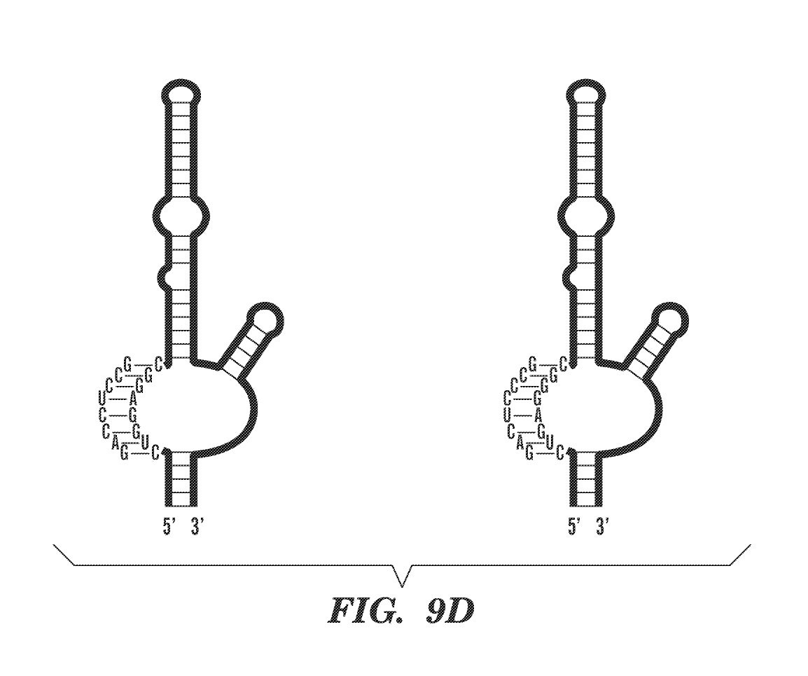

As a proof-of-principle, different RNA constructs of the human telomerase RNA component (hTR) with 2′-O-Me RIPtide microarrays were screened. Telomerase is a specialized ribonucleoprotein composed of two essential components, a reverse transcriptase protein subunit (hTERT), and an RNA component (hTR) (J. Feng, J. Science 269, 1236-1241 (1995)); T. M. Nakamura, Science 277, 911-912 (1997)), as well as several associated proteins. It directs the synthesis of telomeric repeats (5′-TTAGGG-3′) at chromosome ends, using a short sequence within the RNA component as a template. The active telomerase complex purified from human cells consists of three components: the telomerase reverse transcriptase (hTERT), dyskerin, and the telomerase RNA component (hTR), a 451-nucleotide RNA containing the template sequence for repeat addition (S. B. Cohen, Science, 315, 1850-1853 (2007)), as shown in FIG. 9. Several strategies are available for telomerase inhibition, including strategies that target hTR through nucleic acid binding. Some are intended to silence expression; others are directed at the template region and act as competitive inhibitors (C. B. Harley, Nat. Rev. Cancer, 8: 167-179 (2008)).

Telomerase is considered to be an almost universal marker for human cancer, its effect on telomere length playing a crucial role in evading replicative senescence. Evasion of cell cycle arrest through replication-dependent telomere shortening is an adaptation that is believed to be essential for survival of transformed cells. Indeed, whereas in most normal somatic cells telomerase activity is repressed, it has been found that it is activated in approximately 90% of human tumors (J. W. Shay, Eur. J. Cancer 33, 787-791 (1991)); N. W. Kim, Science 266, 2011-2015 (1994)), making inhibition or knockdown of telomerase a strategy for cancer therapeutics.

Existing strategies, however, can still be greatly improved. The size of siRNA molecules poses a challenge for delivery, which may be ameliorated by selecting shorter sequences. Competitive inhibitors focus on the active site for reverse transcription, leaving the remainder of a large complex unexplored—indeed, many other hTR-containing ribonucleoprotein complexes other than the active holoenzyme have been discovered, and these interactions bear interest outside of telomerase catalysis (K. Collins, Mech. Ageing Dev., 129, 91-98 (2008)). To fill this gap, the strategy employed in the studies described herein has been to screen for short nucleic acid sequences, capable of binding hTR, that exert some effect on telomerase activity.

Described herein is the identification of additional targetable sites in hTR that provide unique, interesting, and unexpected alternatives to the template sequence. Of particular interest are sites at which RIPtide binding might interfere with assembly of the telomerase RNP, as such agents are expected to cause rapid onset of apoptosis (Li. S., et al., Cancer Res. 64, 4833-4840 (2004). Folini, M. et al., Cancer Res. 63, 3490-3494 (2003)), rather than the slow onset of senescence that results from inhibition of the mature RNP^{22,27,28}. (Herbert, B.-S. et al., Proc. Natl. Acad. Sci. USA 96, 14276-14281 (1999); Hahn, W. C. et al., Nat. Med. 5, 1164-1170 (1999); Zhang, X., et al., Genes Dev. 13, 2388-2399 (1999)). As described herein, RIPtide microarray screening of a structured element of hTR containing the template and a pseudoknot, both of which are essential for telomerase function, (Mitchell, J. R., Collins, K., Mol. Cell 6, 361-371 (2000)), has resulted in the identification of several new targetable regions in hTR. The RIPtides that target these new sites represent promising candidates for next-generation telomerase inhibitors.

Described herein are methods for RIPtide microarray screening using several hTR constructs within the pseudoknot/template and CR4/CR5 domains of hTR, both of which have been shown to be critical for telomerase activity in vitro and bind hTERT (J. R. Mitchell, Mol. Cell 6, 361-371 (2000)). Reported herein are the setup of the screening platform, hit validation protocols, and anti-telomerase activity, both by in vitro and in cell based TRAP assays, of selected 2′-O-Me RIPtides that bind to human Telomerase RNA.

### Microarray Design Principle

Described herein is the development of a novel microarray platform that provides a structurally unbiased microarray-based screen for RIPtides that bind with high-affinity to a folded RNA target (FIG. 1), and the use of the RIPtides thus identified to modulate telomerase activity in cells. The development of a novel microarray platform that allowed screening for efficient, high-affinity, oligonucleotide-based RNA binders was pursued. The oligonucleotides or RIPtides used for this purpose had to display an improvement in stability, nuclease resistance, and binding affinity compared to standard, unmodified DNA oligonucleotides. It is well-established that the cell permeability of oligonucleotides decreases as a function of length (Loke, S. L., et al., Proc. Natl. Acad. Sci. USA 86, 3474-3478 (1989); Chen, Z., et al., J. Med. Chem. 45, 5423-5425 (2002)), and therefore attention was focused on identifying RIPtides having 8 nucleotides or less. The first approach employed 2′-O-Me oligonucleotides as RIPtide probes to be attached to a microarray surface. 2′-O-alkyl substitution increases nuclease resistance compared to unmodified RNA oligonucleotides and substitution at the 2′ position of the sugar favors the C3′-endo (A-RNA like or North) conformation, which notably increases RNA binding affinity. Moreover, in the context of the RNA target used in this study, 2′-O-methyl oligonucleotides targeted at the template region of hTR have been proven to be efficient telomerase inhibitors (A. E. Pitts, Proc. Natl. Acad. Sci. USA 95, 11549-111554 (1998)); B-S Herbert, Proc. Natl. Acad. Sci. USA 96, 14276-14281 (1999)). Thus, this beneficial modification was incorporated into all of the RIPtides displayed on the microarray.

Relatively short sequences, from 4-mers to 8 mers, were included for the establishment of minimal length requirements for optimal oligonucleotide-RNA binding and to determine whether these short sequences would impact non-canonical base-pairing characteristic of many RNA-RNA interactions. In addition, the use of short sequences allowed, in a single microarray slide, the synthesis of all possible sequence combinations or permutations of the RIPtides, increasing the potential to extend this methodology to the study of RNA of any given sequence.

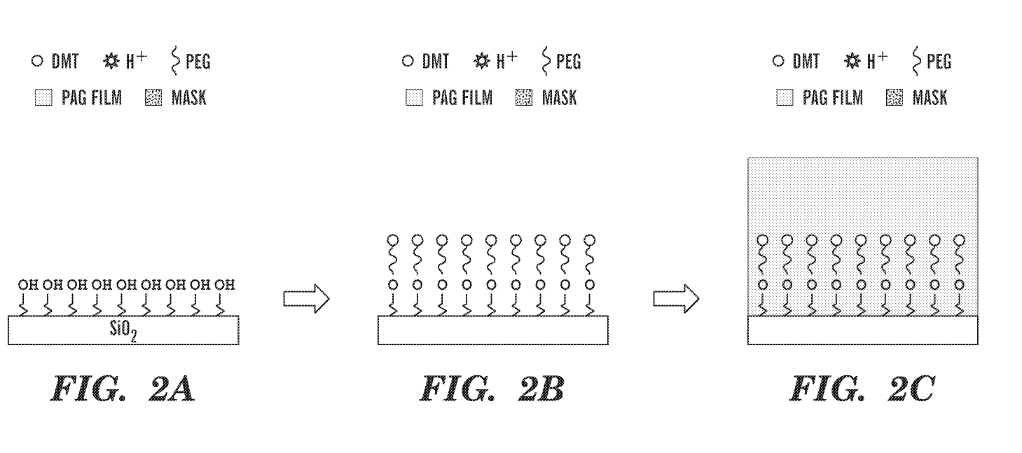

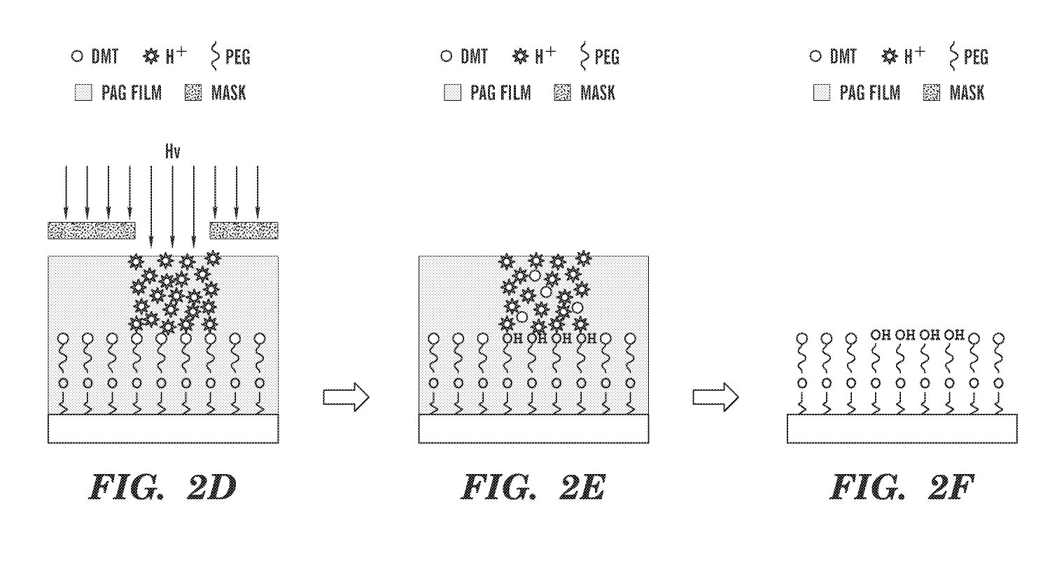

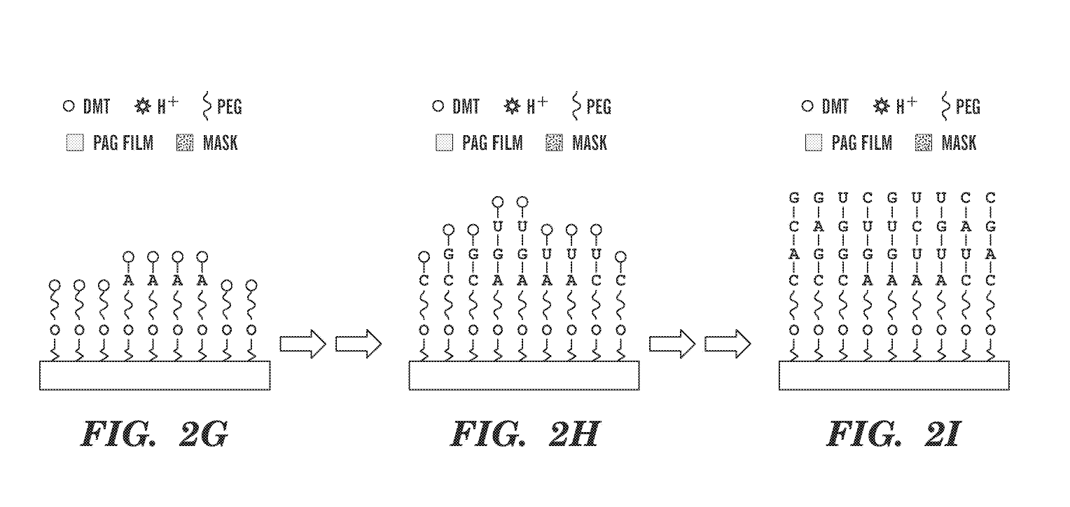

Though 2′-O-methylation was expected to provide substantial performance benefits, it also complicated the fabrication of the microarray, because standard high-density microarray technologies are geared toward 2′-deoxyoligonucleotides. The established Affymetrix platform for photochemically directed microarray synthesis requires the preparation of 5′-photocaged nucleoside 3′-phosphoramidites (Chen, J.-L., et al., Cell 100, 503-514 (2000)), which if applied to the present purpose would have required the synthesis of 5′-photocaged 2′-O-methyl phosphoramidites. The fabrication of the first example of high density 2′-O-Me-RIPtide microarrays as a tool for drug discovery was accomplished by a photoresist technique recently developed by Affymetrix Inc. and based on I-line (365 nm) projection lithography (A. Pawloski, J. Vac. Sci. Technol. B 25, 2537-2546 (2007)). This recently developed microarray fabrication technology employs photochemical generation of an acid capable of deprotecting standard 5′-dimethoxytrityl (DMT) groups (FIG. 2). This methodology is particularly well-suited to the present purpose because it requires only standard, commercially available 2′-O-methyl RNA phosphoramidites, and could in principle be used with any 5′-DMT-protected nucleic acid analog. This photoresist technology (Pawloski, A. et al., J. Vac. Sci. Technol. B 25, 2537-2546 (2007)) allowed us to generate microarrays displaying on each chip all possible 8-, 7-, 6-, 5-, and 4-mer 2′-O-methyl RIPtides having the standard nucleobases A, C, G, and U, a total of 87,296 RIPtides (FIG. 1C). A pre-stainable checkerboard alignment feature was also incorporated into each array.

### Target RNAs

The template/pseudoknot domain of human telomerase RNA (hTR) was used as the RNA target, but it is contemplated that the methods described herein can be used against any RNA target. The template/pseudoknot domain of hTR has a high degree of structural conservation across vertebrates (J. L. Chen, Cell 100, 503-514 (2000)), its core structure being essential for telomerase function (J. R. Mitchell, Mol. Cell 6, 361-371 (2001)). Consistent with this, mutations in this domain give rise to telomerase deficiency diseases in humans, including dyskeratosis congenita and a form of aplastic anemia. RIPtides that bind it, even outside the template region, may exert a functional effect.

The requirement of the formation of a stable, permanent pseudoknot (L. R. Comolli, Proc. Natl. Acad. Sci. USA 99, 16998-17003 (2002); C. A. Theimer, Proc. Natl. Acad. Sci. USA 100, 449-454 (2003); J. L. Chen, Proc. Natl. Acad. Sci. USA 102, 8080-8085 (2005)), versus a transiently formed one, and its implications for telomerase activity have been subject to debate. Several three dimensional structures of engineered minimal pseudoknot RNA's have been reported (Kim, N.-K. et al., J. Mol. Biol. 384, 1249-1261, (2008); Theimer, C. A. et al., Mol. Cell 17, 671-682 (2005); Theimer, C. A. et al., Mol. Cell 27, 869-881 (2007); Theimer, C. A., Feigon, J. Curr. Opin. Struct. Biol. 16, 307-318 (2006)), but apart from this single module of the template/pseudoknot domain, the overall structure remains unelucidated. Recently, the structural features of the domain have been partially revealed (C. A. Theimer, Mol. Cell 17, 671-682 (2005); C. A. Theimer, Mol. Cell 27, 869-881 (2007); C. A. Theimer, Curr. Opin. Struct. Biol. 16, 307-318 (2006)). Interestingly, it has recently been reported that the 2′-OH group of nucleotide A176 in the pseudoknot structure (A176), located distant in primary sequence from the template region, is implicated as making a contribution to the catalytic activity of telomerase (F. Qiao, Nat. Struct. Mol. Biol. 15, 634-640 (2008)).

Screening of the microarray was performed using folded RNA constructs incorporating a fluorescent label, such that the fluorescence intensity of the scanned microarray read out positive RIPtide “hits”. To investigate the extent to which the size of the RNA target influences its ability to access the RIPtides displayed on the microarray, a truncation series was constructed, in some cases using a plasmid construct containing the full sequence of human Telomerase RNA (1-451 nt), representing progressively smaller versions of the template/pseudoknot domain, with the smallest being the 48 nt engineered minimal pseudoknot previously employed by Feigon and co-workers for structural studies (C. A. Theimer, Mol. Cell 17, 671-682 (2005); C. A. Theimer, Mol. Cell 27, 869-881 (2007); C. A. Theimer, Curr. Opin. Struct. Biol. 16, 307-318 (2006), Y. G. Yingling, J Mol Graph Model. 25, 261-274 (2006); Y. G. Yingling, J. Biomol. Struct. Dyn. 24, 303-20 (2007); Y. G. Yingling, J Mol Graph Biol. 348, 27-42 (2005)). Most of these were generated by T7 RNA polymerase-dependent transcription from PCR-generated templates in the presence of small amounts of 5′-aminoallyl-UTP for post-transcriptional labeling by treatment with the N-hydroxysuccinimide (NHS) ester of Cy3 (see Methods); the shortest two were produced by solid-phase synthesis and were 5′-labeled with Cy3. All RNA transcripts were purified by denaturing PAGE, their integrity and size was confirmed by electrophoresis, and they were re-folded as described below.

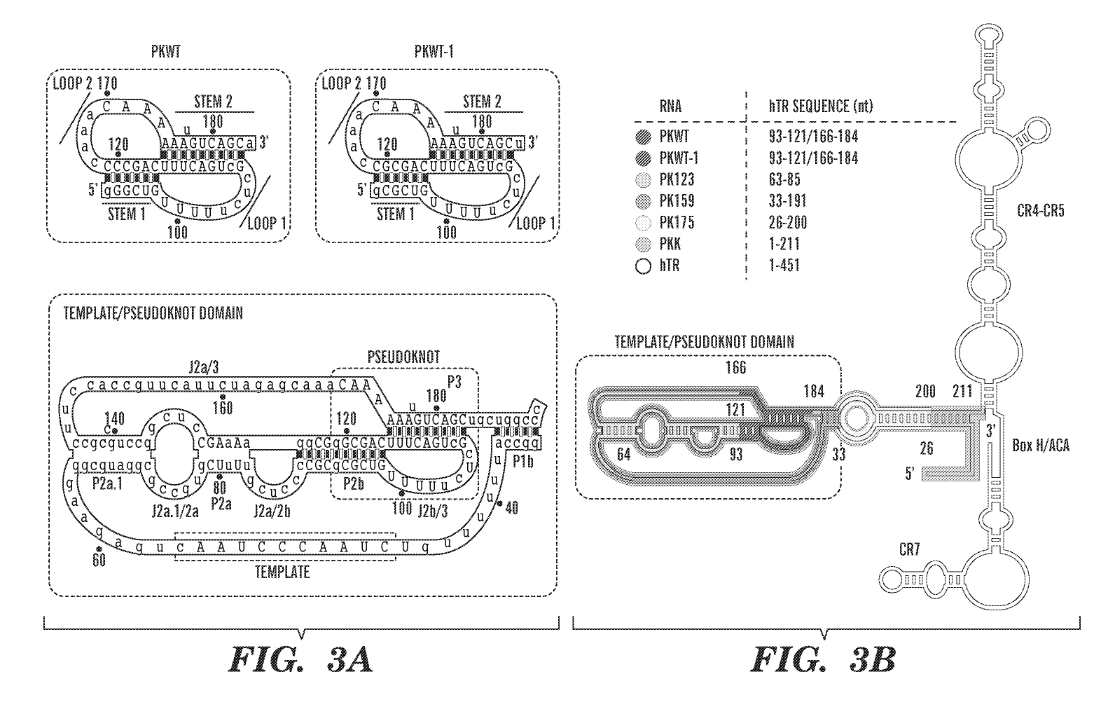

The fluorescently labeled versions of the full-length hTR (nucleotides 1-451) and the template/pseudoknot domain (PKK, nt 1-211) failed to show quantifiable binding to the microarray in an initial screen; and a slightly shorter 175 nt version of the template/pseudoknot domain (PK175, nt 26-200) gave irreproducible results. On the other hand, a 159 nt construct (PK159, nt 33-191) and all shorter versions (FIG. 3B) yielded reproducible microarray positives. It was thus concluded from these initial results that, under the experimental conditions, the 2′-OMe microarrays provide reliable results with RNA targets shorter than ˜160 nt in length, and should be used cautiously with RNA targets longer than this.

Optimization of the microarray screening protocol was thus performed using the engineered minimal pseudoknot constructs and the large RNA transcripts PK123 and PK159. The PKWT and PKWT-1 constructs, encompass the hTR sequence between nucleotide positions 93-121 and 166-184, with an engineered connection between nucleotides 121 and 166 (FIG. 3A). PKWT also contains mutations introduced to stabilize Stem 1 (FIG. 3A) and to increase the efficiency of synthesis using T7 RNA polymerase. PKWT-1 is a variant of PKWT in which one of the mutated base-pairs has been restored back to the wild-type sequence. The high-resolution NMR structure of PKWT, which was recently reported (Kim, N.-K. et al., J. Mol. Biol. 384, 1249-1261, (2008)), reveals a three dimensional fold with extensive tertiary interactions and numerous non-canonical base-pairing interactions.

### 2′O-methyl RIPtide Microarray Screening

For microarray experiments the first step to stain the checkerboard was needed to provide basis for proper grid alignment. This was accomplished by modifying standard hybridization protocols commonly used with the Affymetrix Genechip arrays. Briefly, oligonucleotide B2 at a concentration of 250 pM was hybridized for 16 h at 45° C. using a hybridization cocktail containing buffer and BSA only. Afterward, a staining protocol using streptavidin-phycoerythrin was carried out and chips were scanned. Typically, two rounds of hybridization-staining were needed to obtain optimal fluorescence contrast, although in some occasions one single round proved to be sufficient.

To ensure the existence of folded, secondary structure, all RNAs were refolded by heating and slow cooling to ambient temperature in phosphate buffer containing magnesium (5 mM). Labeled RNAs were incubated with the RIPtide microarrays for varying lengths of time (1, 2, 6, 12 and 18 h), at different temperatures (25 and 37° C.), and at concentrations ranging from 1-100 nM. Experiments performed with RNA larger than 160 nucleotides gave rise to inconsistent results, thereby providing valuable information of the upper limit for RNA hybridization for the microarrays used in this study. Chips were first washed at room temperature with a magnesium containing buffer, followed by a stringent wash to increase the signal-to-noise ratio. This was particularly important for large RNA transcripts, such as PK123 and PK159; for the smaller pseudoknot constructs PKWT and PKWT-1, a mild wash at room temperature was sufficient. Optimized conditions that were found to yield reproducible results with RNA targets of different sizes entailed incubating 100 nM RNA target with the microarray for 1 h at 37° C.; in addition, similar results could be obtained by incubating lower RNA concentrations (≧10 nM), for at least 6 h, at 37° C. With this optimized procedure, replicate microarrays yield nearly identical rankings of high-intensity RIPtide hits.

Following incubation with the target RNA constructs, the RIPtide microarrays were scanned, and the most intense RIPtide “hits” were ranked according to the average raw fluorescence intensity from at least two (normally three) independent microarray experiments. If preferred binding sites for the RIPtides on the target RNA existed, then the RIPtides hits would be expected to fall into clusters having related sequences and target binding sites (as opposed to a random distribution of binding sites). Perl scripts were therefore designed to assess several different potential modes of clustering the hits.

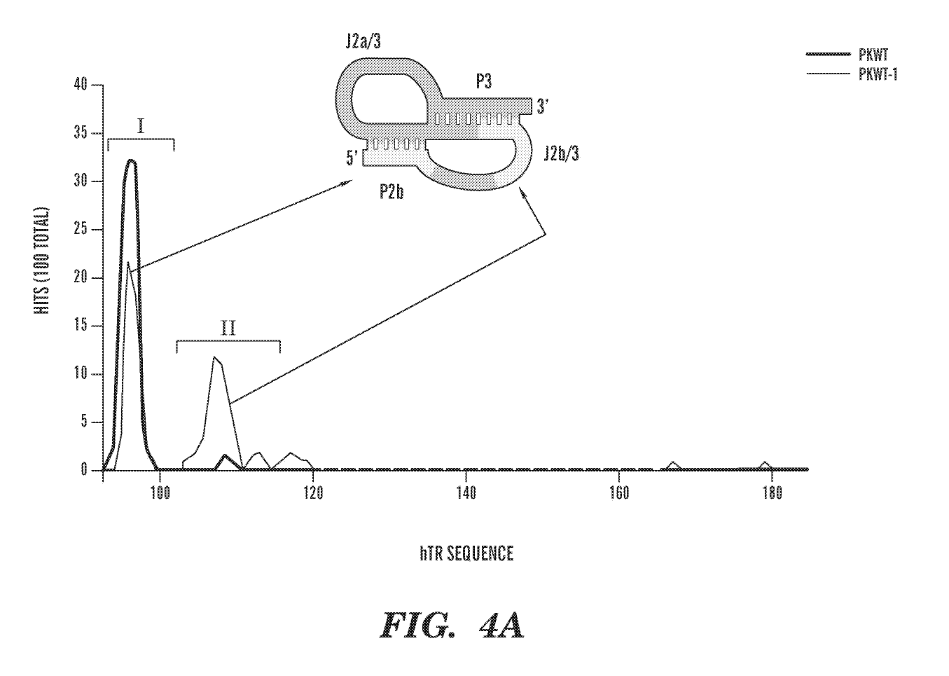

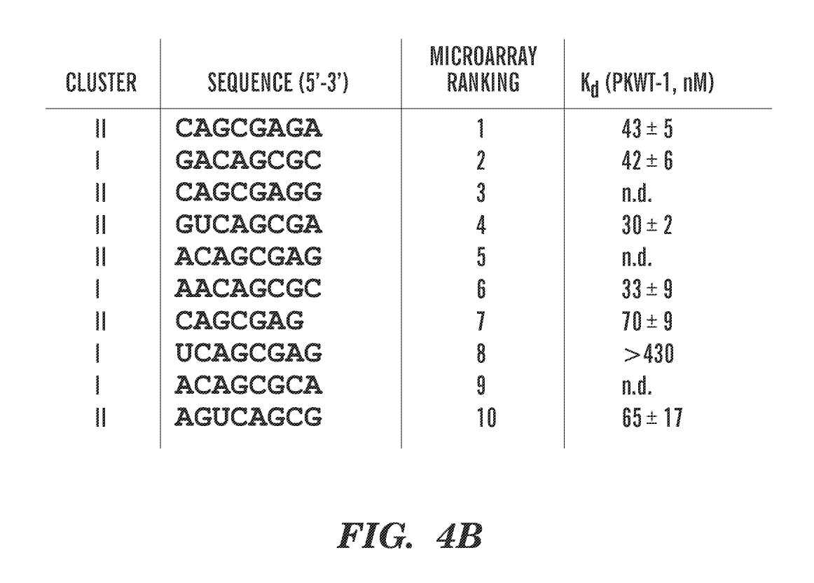

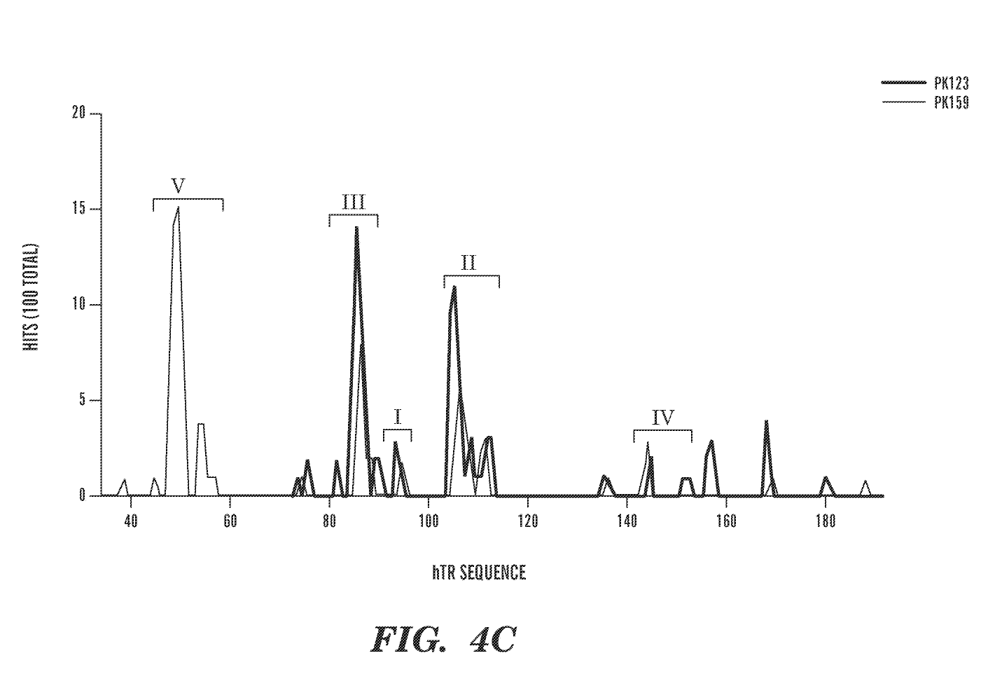

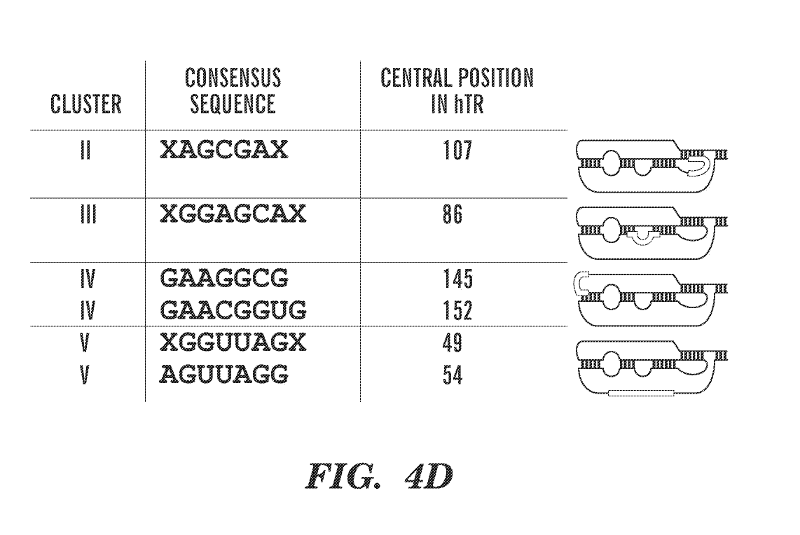

Attempts to cluster the RIPtide hits based solely on their sequence complementarity to one another was found not to produce unambiguously meaningful clusters, because it was difficult with such short sequences to assign a correspondence score to frame-shifted sequences and those having several positions of non-identity. The hits were therefore clustered using their partial sequence complementarity to the RNA target as a guide. In doing so, it was found that RIPtides having non-identical but overlapping sites of partial complementarity with the target could readily be clustered. Specifically, following alignment of the RIPtide hits with the target sequence, a plot of the sites of partial complementarity on the target against the number of hits for each site was constructed (FIG. 4). Only those oligonucleotides having >60% sequence identity to the target RNA were clustered. This clustering provides guidance with respect to tolerated variations among the target binding nucleic acid sequences.

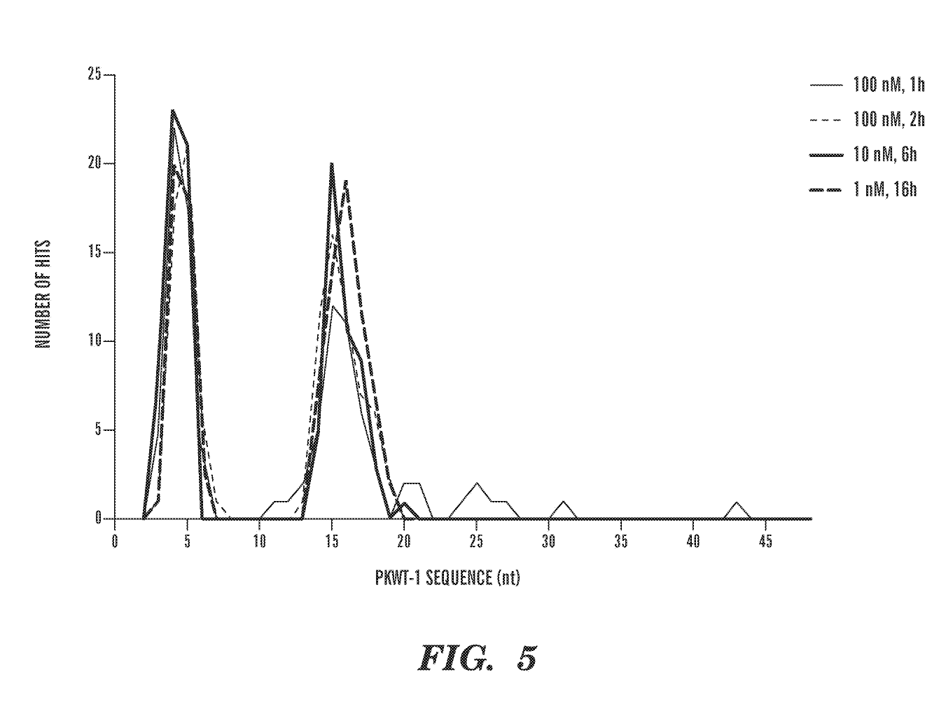

In microarray screens using the engineered pseudoknot constructs PKWT and PKWT-1 (FIG. 4) as targets, the majority of the RIPtide hits exhibiting the highest average fluorescence intensity belonged to a pair of clusters complementary to two regions of the RNA, either the 5′-terminus of the pseudoknot (part of the P2b stem), designated Cluster I, or the J2b/3 loop and an adjacent segment of the P3 stem, designated Cluster II (FIG. 4). Interestingly, though PKWT differs from PKWT-1 at only three nucleotides, a G:C versus C:G base-pair in Stem 1 and the 3′-nucleotide, the two RNA targets show a substantial difference in the relative proportion of hits in Cluster I and Cluster II, indicating that the microarray can be exquisitely sensitive to such subtle sequence changes. In duplex DNA and RNA, the ends are known to undergo more thermal fraying than sites located away from an end, hence the observation of a cluster of apparent binders at the 5′-end was unsurprising. What was unexpected, however, was the nearly complete absence of RIPtides complementary to the 3′-terminus, as the P3 stem in this segment also contains a duplex end. By the same token, it would have been impossible to predict that the J2b/3 loop is so productive for binding to the arrayed RIPtides, while the other loop in the same construct, J2a/3, is almost completely refractory to RIPtide binding. A series of experiments investigating the influence of incubation time on the distribution of the microarray hits was also performed, and it was found that Cluster I emerged more rapidly than cluster II with PKWT-1, but Cluster II continued to accumulate over a longer period of time (FIG. 5).

When larger hTR constructs were subjected to the RIPtide screen (FIG. 4, clustering with PK123 and PK159, overlapped), additional regions on the target apparently amenable to binding were identified. For PK123, Cluster I hits were considerably diminished, though Cluster II remained well-represented, but the most prominent cluster of hits now observed was that complementary to the internal J2a/J2b loop (nt 82-89), designated Cluster III. Several minor clusters at the 5′-end of the J2a/3 single stranded region (nt ˜142-170, including Cluster IV, nt 142-156) were also observed. Finally, when the construct PK159, representing the complete template/pseudoknot domain of hTR, was screened on the 2′-O-methyl RIPtide arrays, a cluster profile similar to that for PK123 was generated, with one major exception: the most prominent cluster observed with PK159 represented RIPtides complementary to the template region (Cluster V, nt 47-57), which was lacking in all the other constructs. The profoundly important role of the template region as the guide sequence for telomere extension requires that it be available for pairing, and indeed a substantial body of literature documents the targetability of the template region by oligonucleotides. The microarray results corroborate these findings, indicating that of all the sites in the PK159 pseudoknot/template construct, the template region is the most productive site for targeting by RIPtides.

### In vitro Validation of the RIPtide Microarray Hits

To assess and quantify the ability of the RIPtide hits from microarray screening to bind the target RNA in solution, a panel of RIPtides representing variations on the consensus sequences of top hits within each cluster was selected. These RIPtides were synthesized with a 3-carboxyfluorescein (FAM) label attached to the 3′-end, the same as had been attached to the surface of the microarray. Fluorescence polarization (FP) was then used to measure quantitatively the equilibrium dissociation constant (K_{d}) values of the FAM-labeled RIPtides, using the same folded target RNAs and buffer system as had been employed in the microarray screen.

A representative sample of the top 10 RIPtide hits from the PKWT-1 screen was first selected, and the affinity of the corresponding interaction in solution was measured. As seen in FIG. 4B, all but one of the top 10 RIPtides bound PKWT-1 in solution with a K_{d} below 100 nM, and a rough correlation between rank order in the microarray screen and affinity for PKWT-1 was observed, with RIPtides of lower rank generally having lower affinity for PKWT-1 (higher K_{d} values). It was also observed, as had been seen in the primary microarray screen, that fully complementary 8-mers generally bound PKWT-1 more tightly than 7-mers resulting from end truncation of a single nucleotide, which in turn bound more tightly than truncated 6-mers, and that fully complementary oligonucleotides generally bound more tightly than those having a single mismatch. These trends are fully consistent with expectation based on established pairing thermodynamics, and validate the use of RIPtide microarrays to identify high-affinity binders to a folded RNA target.

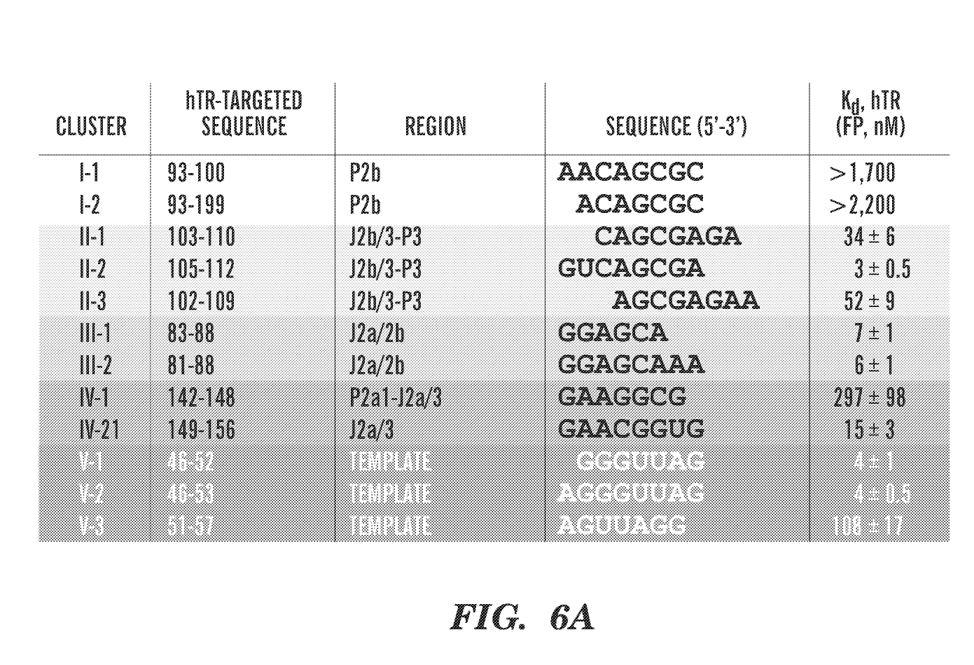

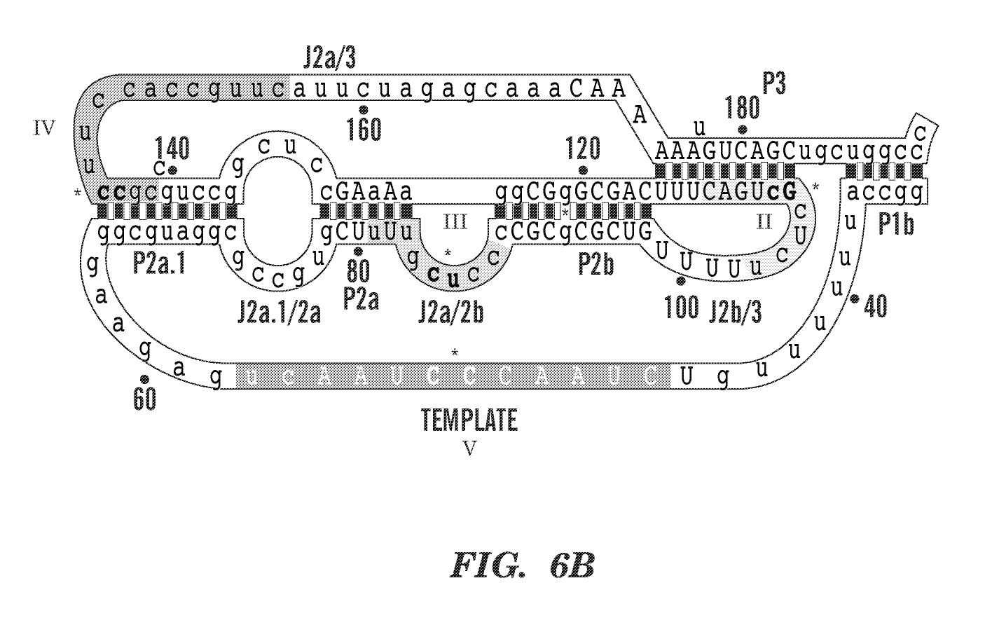

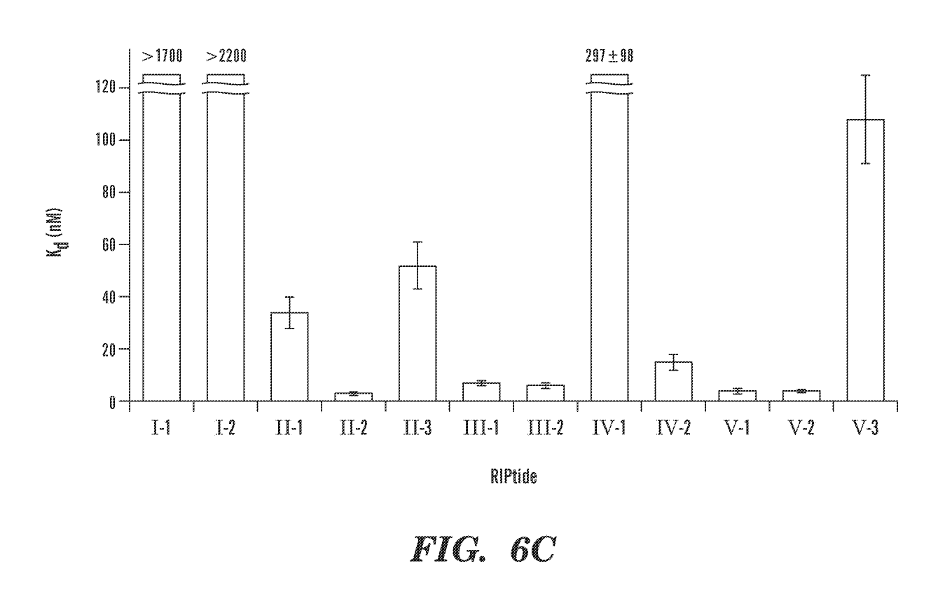

It is possible, without wishing to be limited by a theory, that RIPtide binding sites present or available in truncated forms of hTR may not be present or available in full-length hTR. Five RIPtides were therefore selected that had been validated for binding PKWT-1 in solution, and their binding affinity to full-length hTR was measured using FP. As seen in FIG. 6, none of the Cluster I hits showed any measurable affinity for hTR, whereas the Cluster II hits showed at least as high an affinity for hTR as for PKWT-1, and one RIPtide (II-2) even showed an improvement in affinity. It was hypothesized, without wishing to be bound or limited by theory, that the Cluster I hits became inactive because the end of the pseudoknot to which they bind in PKWT-1 is highly engineered and therefore markedly divergent from hTR; on the other hand, the J2b/3 loop to which the Cluster II hits bind is retained in full-length hTR. Were the J2b/3 loop involved in tertiary interactions in hTR, RIPtide binding might have been lost, and therefore it was surmised that the loop remains relatively unengaged in such interactions when present in naked hTR.

The remainder of the RIPtide hits from primary microarray screens of PK123 and PK159 in solution were not validated, but instead validation using full-length hTR was analyzed. Representative examples from each of the clusters were selected (FIG. 4D) and the binding affinity of these RIPtides for full-length hTR was quantified (FIG. 6A). In this way, RIPtides from clusters III, IV and V that bind full-length hTR were identified. Taken together, the collection of hTR-validated RIPtides maps out a series of sites on the template/pseudoknot that are especially conducive to targeting by a 2′-O-methyl polyribonucleotide; with each site corresponding to a cluster of sequence-complementary RIPtides (FIG. 6B, shaded according to sequence in FIG. 6A). Specifically, these hyper-targetable regions are the J2b/3 loop and P3 stem (Cluster II), the J2a/2b bulge through part of the P2a stem (Cluster III), the J2a/3 loop (Cluster IV), and the Template region (Cluster V). It is noted that all of them are suggested by the hTR folding diagram to have at least some single-stranded content. That said, other prominent tracts suggested by the folding diagram to have single-stranded content are further noted, such as the entire 3′-end of the J2a/3 loop and the J2a.1/2a bubble, which do not appear to be available for targeting by RIPtides.

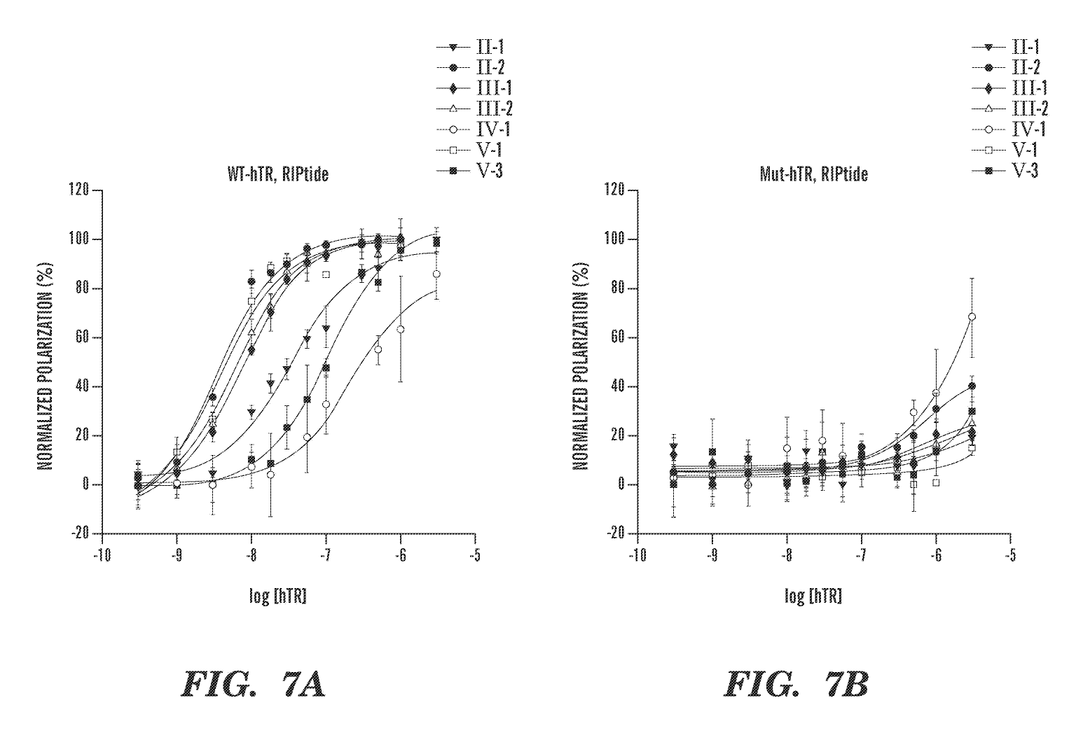

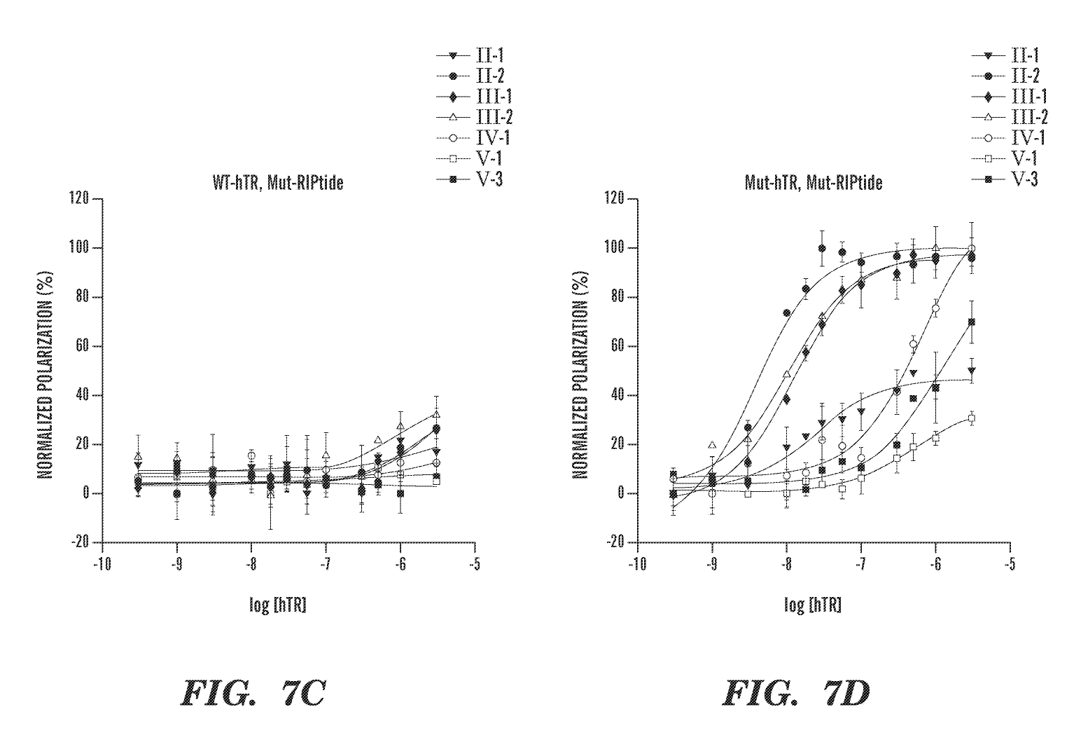

Without wishing to be limited or constrained by theory, the RIPtide binding sites on hTR had been inferred assuming Watson-Crick complementarity between the RIPtide and target. To verify experimentally that the RIPtides were actually recognizing the predicted regions on hTR, tandem point mutations were introduced into the central portion of the RIPtides and compensatory sequence changes into hTR. The binding behavior of the “wild type” and “mutant” RIPtides to wild-type and compensatory mutant hTR targets was analyzed by FP (FIG. 7). Four different hTR transcripts were generated in which two consecutive nucleotides at the central position of each cluster, the expected target site (FIG. 6A, bases indicated in bold), were mutated to their Watson-Crick complementary bases (G→C, C→G and U→A). In each case, binding of the mutated hTR to the “wild-type” RIPtide was abolished or severely reduced (compare FIG. 7A with FIG. 7B). Similarly, binding was abolished or reduced when mutated RIPtides were incubated with wild-type hTR (FIG. 7C). When compensatory mutations were introduced into both the RIPtide and hTR (compare FIG. 7A with FIG. 7D), binding was partially or fully restored in most cases, confirming the site targeted by the RIPtide. Restoration was not observed in two of the seven cases (V-1 and II-1), though restoration was observed with RIPtides that bind an overlapping target site (V-3 and II-2). Perhaps this lack of restoration in certain cases reflects a local change in the availability or in the folding energy of the single-stranded elements as a result of the mutation. Taken together, this mutational specificity supports the notion, without wishing to be bound by theory, that the RIPtides indeed target telomerase at the corresponding sequence-complementary sites.

### Evaluation of Telomerase Inhibition by RIPtides in vitro and in Cultured Cells

Having discovered a panel of RIPtides that bind four different regions on the naked RNA component of telomerase, it was next determined whether these molecules were capable of inhibiting the activity of the telomerase ribonucleoprotein complex in an in vitro setting. The Telomeric Repeat Amplification Protocol (TRAP) assay (Kim, N. W. et al., Science 266, 2011-2015 (1994)) was therefore employed. The TRAP assay is a PCR-based protocol that has found widespread use in determining telomerase activity in human cell extracts and also in evaluating the in vitro potency of telomerase inhibitors. Using a version of the TRAP assay (Cy5-TRAP) that utilizes fluorescence detection (Herbert, B.-S. et al., Nat. Protocols 1, 1583-1590 (2006)). IC₅₀ values for several RIPtides were determined using cell extracts from two human tumor cell lines (HeLa and DU145) and an immortalized embryonic cell line (HEK293). Initially, a small library of RIPtides representing several clusters identified in the microarray screen was screened and validated by FP experiments on hTR, using telomerase activity present in HeLa cell extracts. The majority of these were 8-mers, but some 7-mers and 6-mers were also tested; all were fully complementary to the target hTR sequence with K_{d}'s for hTR below 300 nM. Several phosphorothioate variations of the initial library were additionally tested, incorporating phosphorothioate linkages either at the two terminal positions of the RIPtide or at every position.

In the first round of screening experiments, and for the phosphodiester compounds, inhibitory activity was found in two examples of 8-mer RIPtides complementary to the template (Cluster V, SEQ ID NO:26). No significant inhibition by compounds belonging to clusters II, III and IV was observed. For the phosphorothioate derivatives, several RIPtides tested from clusters II, III and V exhibited telomerase inhibition in the 1-10 μM concentration range; with RIPtides targeting the template having the lowest IC₅₀ values of the series, ˜1-2 μM

In an attempt to increase the potency of certain RIPtides that showed some inhibitory activity in the TRAP assays, their length was increased by 2-3 nucleotides at either end, maintaining Watson-Crick pairing with hTR. This strategy did not improve the activity of RIPtides by Cluster II or Cluster III, suggesting, without wishing to be bound or limited by a theory, that in the assembled ribonucleoprotein complex the regions of hTR recognized by these RIPtides may be kinetically inaccessible, or alternatively, that the protein component of telomerase thermodynamically out-competes the RIPtide for that site on hTR. However, RIPtides of different lengths targeting the alignment sequence in the template region (Cluster V) were effective telomerase inhibitors. Moreover, it was also found that several sequence-extended versions of Cluster IV RIPtides, which target the 5′-end of the J2a/3 loop, exhibited nanomolar IC₅₀ values in TRAP assays with cell lysates. Oligodeoxynucleotides targeting the same region have been previously reported and demonstrated to have inhibitory activity against telomerase in vitro; however, no criteria for having selected that particular site were described (Pruzan, R., et al., Nucleic Acids Res. 30, 559-568 (2002)). The RIPtide mapping experiments reported herein establish that this particular site is especially productive for targeting in naked hTR, but unlike several other sites thus identified, it remains targetable in the fully assembled form of telomerase. Most importantly, targeting at the accessible Cluster IV site produces potent inhibition of telomerase enzyme activity in vitro.

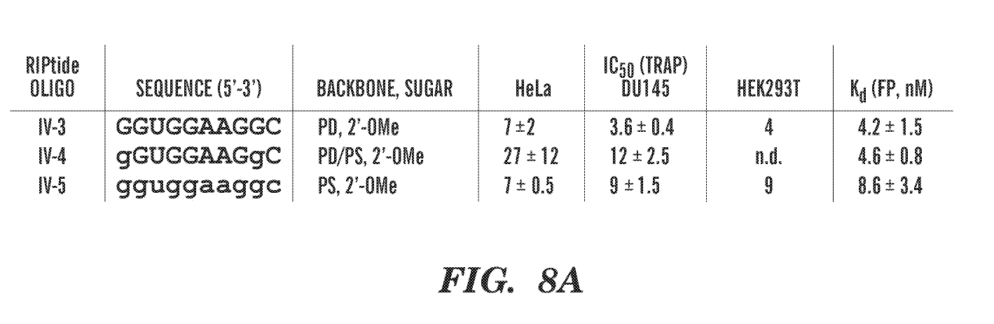

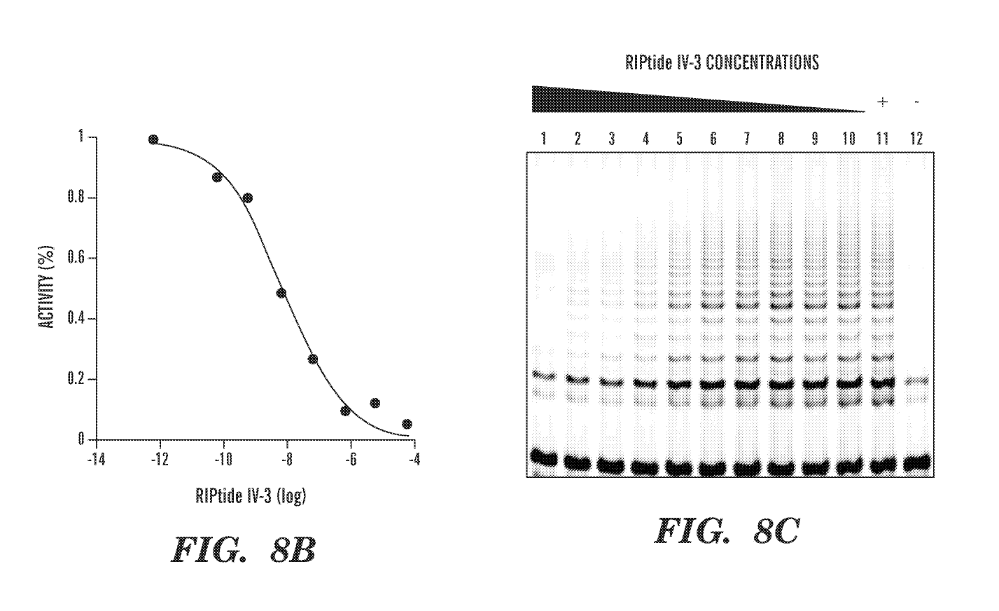

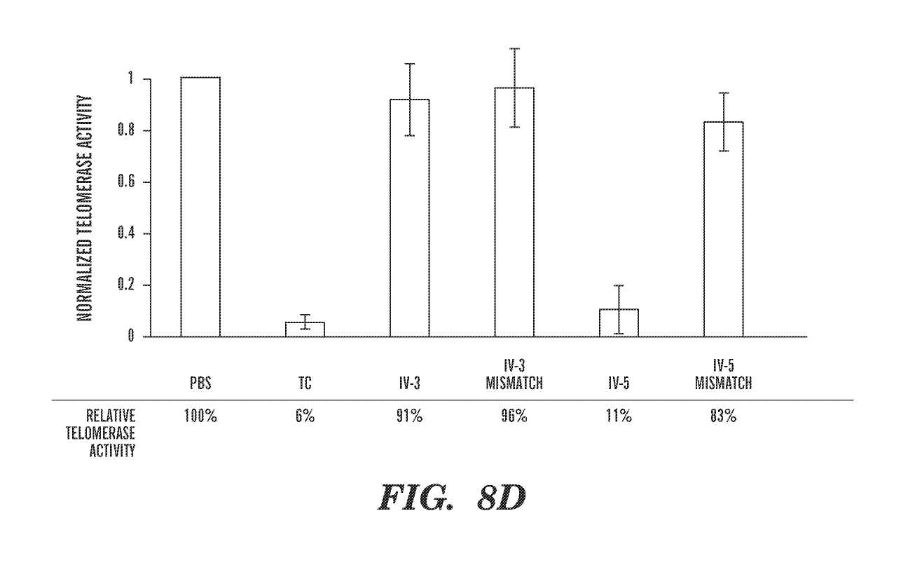

Optimization of RIPtides that target this site was performed starting from a 14-mer covering hTR sequence 143-156 nt, followed by serial truncations on either end, until a minimal sequence was identified comprising 10 nucleotides (complementary to hTR 143-152 nt, entry 32), from which removal of additional bases abolished telomerase inhibition in vitro. All RIPtide sequences that included this minimal sequence and possessing a length of 10 nucleotides or longer inhibited telomerase activity with an IC₅₀ below 10 nM. Thus, through a combination of the novel RIPtide microarray screening and systematic extension guided by TRAP assays, a novel and unique minimal sequence that produces telomerase inhibition at low nanomolar concentrations in vitro was identified. This sequence, (SEQ ID NO: 20) 5′-GGUGGAAGGC-3′ (IV-3), inhibited telomerase activity present in all cell lines tested, with IC₅₀ values in the low nanomolar range (FIG. 8).

Furthermore, in parallel efforts aimed at obtaining RIPtides with better pharmacologic profiles for cell-based activity assays, such as increased stability versus nucleases and/or increased RNA binding affinity, the chemistry of the most promising inhibitory sequence was modified and the telomerase inhibitory potential of RIPtides which include different modifications at the backbone were explored. As the 10-mer RIPtide described above might have insufficient stability or cell permeability to inhibit telomerase activity in cultured cells, a screen incorporating chemical modifications known to increase stability, cell permeability and binding potency was performed, while monitoring the retention of activity in vitro using TRAP assays. Specifically, the effect of phosphorothioate substitution and replacement of the 2′-O-methyl-ribose backbone with the locked nucleic acid (LNA) backbone on telomerase inhibition in TRAP assays was assessed. Phosphorothioate substitutions were made at either the 5′-most and 3′-most phosphodiester groups, or at every phosphate linkage. In both cases, the phosphorothioate-substituted RIPtides retained their ability to inhibit telomerase activity, exhibiting IC₅₀ values in the low nanomolar range (FIG. 8A, RIPtides IV-3 (SEQ ID NO:20), IV-4 and IV-5). Moreover, the IC₅₀ values were found to be in good agreement with the K_{d} values determined by fluorescence polarization experiments (FIG. 8A-8C). For both the phosphodiester and phosphorothioate 2′-O-methyl RIPtides, mismatch-containing RIPtides were used as negative controls to rule out non sequence-specific effects (FIG. 8D). This is crucial in establishing sequence specificity for nucleic acid-based drugs, but is especially necessary in the case of phosphorothioates, as phosphorothioates have previously been reported to bind to hTERT in a non-specific manner (Matthes, E., Lehmann, C., Nucleic Acids Res. 27, 1152-1558 (1999)). It was found that telomerase inhibition by RIPtides containing mismatches was completely abolished, establishing the sequence specificity of the observed results. In addition, a single RIPtide of the 10-mer sequence with an entirely LNA backbone was also tested, and the inhibitory potency was found to be ˜1 nM.

Having established that the modified RIPtides retain activity in vitro, several of these were tested in cell-based assays. DU145 prostate cancer cells were treated with 165 nM RIPtide for 24 h. The cells were subsequently lysed and telomerase activity was assessed by the TRAP assay (FIG. 8D). As a positive control, a previously reported 13-mer 2′-O-methyl oligonucleotide targeting the template region of hTR was employed (Pitts, A. E., Corey, D. R., Proc. Natl. Acad. Sci. USA 95, 11549-111554 (1998)). Lipofectamine™ was used to ensure optimal delivery, and it remains to be established whether cationic lipid delivery is necessary for 10-mers. In particular, there is evidence that relatively short oligonucleotides containing phosphorothioate linkages targeting telomerase show optimal cellular uptake properties (Chen, Z., et al., J. Med. Chem. 45, 5423-5425 (2002)). While cells treated with RIPtide SEQ ID NO:20, having a phosphodiester backbone and a 2′O-methyl sugar, showed no significant telomerase inhibition, RIPtide SEQ ID NO:20, having a phosphorothioate backbone and a 2′O-methyl sugar did produce marked inhibition of telomerase, possibly reflecting the greater cell-permeability and stability of the latter. Importantly, introduction of two point mutations into RIPtide SEQ ID NO:20, having a phosphodiester backbone and a 2′O-methyl sugar, known to abolish telomerase inhibition in extract-based experiment, also abolished inhibition in these cell-based experiments, supporting a sequence-specific mechanism of inhibition by RIPtides. This is of especial relevance as this is the first example of an oligonucleotide targeting this region having demonstrated inhibition of telomerase activity in cultured cells.

### Discovery of Inhibitory Sequences In Vitro

Another aspect has focused on nucleotide sequences directed at the CR4-CR5 domain of hTR, as seen in FIG. 9, one of two domains required for activity in vitro (F. Bachand, Mol. Cell. Biol., 21, 1888-1897 (2001)).

In seeking in vitro inhibitors of telomerase, the process followed a typical drug discovery progression: unbiased screen for lead molecules, KD determination, and IC50 determination in an in vitro activity assay. In this case, a 2′-O-methyl oligonucleotide microarray was used to screen for lead oligonucleotide sequences; KD was determined by fluorescence polarization (FP); and effect on telomerase activity was assessed using the telomeric repeat amplification protocol (TRAP).

All permutations of 2′-O-methyl nucleotide sequences from 4- to 8-mers were printed on microarray chips by Affymetrix. An 84-nucleotide construct was synthesized composing the CR4-CR5 domain of hTR by in vitro transcription, and the construct labeled with Cy3. The fluorescently-labeled construct was then allowed to hybridize on the microarrays, and the chips were scanned for fluorescent hits. These hits were categorized by sequence consensus, and binding sites were predicted based on sequence complementarity. It was found that the 100 brightest spots on the microarrays could be clustered into four putative binding sites on the CR4-CR5 domain, as seen in FIG. 9C. These clusters represent regions predicted to comprise loops (J. L. Chen, Cell, 100, 503-514 (2000)).

To determine binding affinity in solution, an unlabeled version of the same 84-nucleotide construct by in vitro transcription was synthesized. Also synthesized were fluorescein-labeled 2′-O-methyl oligonucleotide sequences corresponding to intensely fluorescent spots from the microarray screen. K_{D} was determined by fluorescence polarization measurements. Representatives from each cluster were screened and found that out of four sites available for binding as determined by microarray analysis, only two were confirmed by FP (Table 3).

Inhibition of telomerase activity in vitro was determined using TRAP, a PCR-based assay for telomerase activity in cell extracts (B.-S. Herbert, Nat. Protocols, 1, 1583-1590 (2006)). Unlabeled oligonucleotide sequences found to bind by FP were pre-incubated with cell extracts (HeLa, DU 145, and 293), and activity was measured by TRAP. Out of the sequences tested, only one, SEQ ID NO: 1, was found to inhibit telomerase activity, with an IC50 in the micromolar range (Table 2). As SEQ ID NO: 1 is predicted to bind in the J5/6 loop, as seen in FIG. 9D, a region otherwise relatively unexplored for telomerase inhibition, it may belong to a novel class of telomerase inhibitor.

### Confirming In Vitro Mechanism of Action

The working hypothesis was that SEQ ID NO: 1 binds to the J5/6 loop on CR4-CR5, and that this binding event inhibits telomerase activity as observed by TRAP. If this is true, the discovery of SEQ ID NO: 1 raises questions about the significance of the J5/6 loop, a region on hTR not previously associated with necessity for telomerase activity (J. R. Mitchell, Mol. Cell, 6, 361-371 (2000)). Thus, it is crucial to gather supporting evidence for these assumptions by doing compensatory mutation experiments, as represented in FIG. 9D.

Previous FP experiments were performed on wild-type hTR in vitro transcribed products. If two nucleotides on hTR internal to the predicted binding site were swapped, it is expected that binding to SEQ ID NO: 1 would be lost. If instead an oligonucleotide with the compensatory mutations is added, binding with a similar KD would be restored. Mutant plasmid constructs of hTR have been made, and mutant hTR has been in vitro transcribed the mutant hTR. Next, a fluorescein-labeled oligonucleotide with the compensatory mutations can be synthesized and tested by FP, to demonstrate that the initial FP data describes a specific binding event between SEQ ID NO: 1 and J5/6.

To confirm whether a binding event to the J5/6 loop on hTR is correlated with loss of telomerase activity in vitro, VA13 cells (which express neither hTR nor hTERT) may be used, and have previously been used to perform a number of mutational studies on hTR. Similar to the FP experiments, the ability of an oligonucleotide with the compensatory mutations to inhibit activity of a mutant telomerase holoenzyme can be tested by TRAP. For this, a plasmid construct of hTR has been prepared and site-directed mutagenesis performed in the predicted SEQ ID NO: 1 binding site. Several different mutation combinations can also be tried in order to prevent loss of telomerase activity through mutation alone.

### Testing in Cells

Major questions that result as a consequence of these analyses are directed towards whether cells treated with discovered oligonucleotides show decreased telomerase activity, and whether prolonged treatment results in telomere shortening and cell cycle arrest. Implicit in these questions are problems universal to oligonucleotide therapeutics: nuclease stability and delivery across the cell membrane (I. Lebedeva, Ann. Rev. Pharmacol. Toxicol., 4, 403-419 (2001)). Several diverse backbone modifications have been shown to increase stability to exonucleases, and the modified monomers for nucleic acid synthesis are commercially available.

Sequences discovered from microarray analysis tended to be 6- to 8-mer sequences clustered around certain consensus sequences, thought to correspond to site of binding. Several sequences from each cluster were assayed for binding, and the range of KD values obtained are summarized, with lower KD values usually corresponding to the longest sequences with highest complementarity. Binding affinity was initially measured with a construct only representing the CR4-CR5 domain, and binding affinity of SEQ ID NO:1 was confirmed on a full-length construct. Sequences from Clusters 1 and 4 were assayed by TRAP, with only one sequence (GCCUCCAG, or SEQ ID NO:1) showing inhibition of activity. Clusters 2 and 3 did not show binding by FP, and were not assayed by TRAP. A sample of several oligonucleotides synthesized to increase SEQ ID NO:1's nuclease resistance. Asterisks indicate the presence of the corresponding modification on the backbone. KD values were determined with a full-length hTR construct.

Phosphorothioate backbones are known to increase nuclease resistance (I. Lebedeva, Ann. Rev. Pharmacol. Toxicol., 4, 403-419 (2001)), and also render oligonucleotides more cell permeable (G. D. Gray, Biochem. Pharmacol., 53, 1465-1476 (1997)). Phosphorothioate modification can also reduce helix stability, and while several versions of SEQ ID NO: 1 with phosphorothioate modifications have been made, inhibition by TRAP is preserved only with single modifications at either terminus, with IC50 values on the order of 10 μM, as seen in Table 2. A variant of SEQ ID NO: 1 (termed SEQ ID NO: 1 L) was synthesized with a locked nucleic acid backbone, a modification that increases nuclease stability as well as duplex melting temperature (H. Kaur, Chem. Rev., 107, 4672-2697 (2007)). SEQ ID NO: 1 L also shows telomerase inhibition by TRAP, with an IC50 similar to that of 2′-O-methyl, all-phosphodiester SEQ ID NO: 1 (Table 2).

The issue of delivery across the cell membrane can be temporarily circumvented by lipofecting cultured cells with oligonucleotides. Once it is established that SEQ ID NO: 1 variants are capable of telomerase inhibition after transfection into cultured cells, methods of delivery that can retain as much efficacy as possible can be explored. In order to determine whether any SEQ ID NO: 1 variants show inhibitory effects in cultured cells, short-term treatment experiments can be performed, in which cultured tumor cells are transfected with oligonucleotide, and then assayed for telomerase activity after a short period of time (B.-S. Herbert, Proc. Natl. Acad. Sci. USA, 96, 14276-15291 (1999)).

The oligonucleotide variants capable of inhibiting telomerase activity soon after transfection can be carried into longer-term treatment studies, in which continuoustreatment occurs for several weeks, with periodic checking for cell proliferation, and measuring average telomere lengths over time (M. R. Alam, Nucleic Acids Res., 36, 2764-2776 (2008)). In parallel, delivery can also be optimized. After determining the permeation capabilities of the oligonucleotides alone, lipids (C. B. Harley, Nat. Rev. Cancer, 8, 167-179 (2008)), peptides (M. R. Alam, Nucleic Acids Res., 36, 2764-2776 (2008)), or small molecule/drug moieties (W. M. Flanagan, Nat. Biotechnol., 17, 48-52 (1999)) can be added to promising oligonucleotide variants.

### Target RNA Sample Preparation

Human telomerase pseudoknot constructs PKWT and PKWT1 with a dye label at the 5′-end (Cy3 or DY-547) were purchased from Dharmacon. All RNA fragments longer than 50 nt were obtained by run-off in vitro transcription from a dsDNA template generated by PCR from a pRc/CMV vector containing hTR48 using appropriate primers and in the presence of aminoallyl-UTP. In vitro transcription was performed at 37° C. overnight using purified His6-tagged (SEQ ID NO: 55) T7-RNA polymerase in the presence of 4 mM NTPs, 1 U/mL yeast inorganic pyrophosphatase, RNase inhibitor, and 10× transcription buffer (400 mM Tris, pH 8, 100 mM MgCl₂, 50 mM DTT, 10 mM spermidine and 0.1% Triton X-100). After DNase I treatment (15-30 min, 37° C.), ethanol precipitation, and purification by denaturing polyacrylamide gel electrophoresis (PAGE), the target RNA was labeled with Cy3-NHS ester (Amersham, 0.1M Na2CO3, pH 8.5, 50% DMSO/H2O, 1 h). Excess dye was removed by ethanol precipitation and labeled RNA was purified by denaturing PAGE in 1×TBE (90 mM Tris-borate, 2 mM EDTA) buffer and subsequent desalting. RNA purity, yield, and ratio of incorporated dye per RNA molecule were determined by optical (OD) measurements at wavelengths 260, 280 and 550 nm and by agarose gel electrophoresis with ethidium bromide staining.

### Microarray Hybridization and Data Analysis

To facilitate analysis, the RIPtide chips included four areas delimiting the 2′-O-methyl array that, when stained with a specific probe (oligo B2, Affymetrix), would display a visual “checkerboard” as a grid alignment guide. This was accomplished by modifying standard hybridization protocols commonly used with the Affymetrix Genechip arrays. Briefly, 250 pM oligonucleotide B2 was hybridized to the checkerboard for 16 h at 45° C. using a hybridization cocktail of buffer and BSA. Afterward, probes were stained using streptavidin-phycoerythrin and the chips scanned. Typically, two rounds of hybridization-staining were needed to obtain optimal fluorescence contrast, although occasionally one single round proved to be sufficient.

A solution of folded Cy3-labeled RNA was heated at 95° C. for 3 minutes and slowly cooled to 37° C. in 1× array buffer containing magnesium (final concentration 50 mM potassium phosphate, 150 mM KCl and 5 mM Mg(OAc)₂, pH 7.4). Checkerboard-stained microarrays were pre-incubated with 1× array buffer at 37° C. for 30 minutes prior to RNA addition. Concentrations of folded RNA used in these experiments varied from 1-100 nM, with incubations at 37° C., for 1-16 h. 16 h experiments were carried out for controls under hybridization conditions. The arrays were then washed with 1× array buffer and scanned using the Affymetrix Genechip 3000 7G scanner. To increase the signal-to-noise ratio, an additional, more stringent wash was used.

Microarray images were analyzed using GCOS (Genechip Operating Software, Affymetrix Inc.). Background fluorescence was qualitatively evaluated by scanning the arrays prior to target RNA incubation. Results were visualized with Spotfire (TIBCO) or Rosetta Resolver (Rosetta) softwares. Initial fluorescence-based ranking of RIPtides was carried out with Microsoft Access. Maximum fluorescence values for replicate experiments were compared, and no normalization was considered necessary at this step.

After raw fluorescence values were averaged, a list of the top 100 hits was extracted using Perl scripts developed in-house. The RIPtide sequences were aligned against the target RNA sequence to identify putative binding sites.

### Fluorescence Polarization

FAM (6-carboxyfluorescein)-labeled oligonucleotides were synthesized on a 3′-(6-Fluorescein) CPG support (Glen Research) using a MerMade 12 (BioAutomation) DNA synthesizer, purified with Poly Pak-II (Glen Research) cartridges, and compositionally verified by MALDI-TOF MS. Unlabeled full length hTR was prepared by in vitro transcription in the presence of T7 RNA polymerase under the conditions described earlier for RIPtide screening, but without aminoallyl-UTP addition. After DNase I treatment and ethanol precipitation, hTR was purified using the RNeasy Midi kit (Qiagen). Unlabeled PKWT and PKWT-1 were purchased from Dharmacon, and were PAGE-purified and desalted. FAM-labeled RIPtides (5 nM) were titrated with increasing concentrations of folded RNA (300 pM-3 μM, typically). Solutions containing RIPtide and RNA were incubated at 37° C. for 2 h, after which fluorescence polarization was recorded at room temperature using a SpectraMax M5 (Molecular Devices) plate reader. Polarization (expressed in millipolarization units) was monitored at 485 nm with excitation at 525 nm (cutoff 515 nm). Negative controls employed in the assay included all 2′-O-Me 8-mer A, C, G and U homopolymers, a FAM linker with no nucleic acid attached, and mismatch-containing RIPtides as described in the text. Dissociation constants were determined using Kaleidagraph 3.5 (Synergy Software). Triplicate experiments were fit to the following equation: (m1+(m2−m1)/(1+10̂(log(m3)−x)); m1=100; m2=0.1; m3=0.0000003.

For mapping of hTR-RIPtide binding sites, site-directed mutagenesis on the pRc/CMV plasmid (Collins lab, UC Berkeley) was performed using a QuickChange-XL mutagenesis kit (Stratagene) and confirmed by sequencing. Full-length hTR transcripts incorporating two consecutive base mutations (to their Watson-Crick complementary bases) were generated for fluorescence polarization studies.

### TRAP Activity Assays

RIPtides were synthesized, purified with PolyPak-II C18 reverse phase cartridges, and constitutionally verified by MALDI-TOF MS. Telomerase-positive cells were either purchased from ATCC (DU145 and HEK293) or provided in the Chemicon TRAP kit (HeLa). Cell extracts were prepared from cell pellets by detergent lysis with 1×CHAPS lysis buffer (Chemicon). RIPtides were incubated with cell extract for 1 h at 37° C. prior to the TRAP assays. Assays were performed following a protocol that uses fluorescence as a quantitation system, as previously described by Herbert et al. (Nat. Protocols 1, 1583-1590 (2006)). Briefly, extension of a fluorescent artificial substrate by telomerase was carried out for 30 minutes at 30° C., followed by amplification with 30 PCR cycles (34° C. 30 s, 59° C. 30 s, 72° C. 1 min). Telomerase extension products were separated on 10% native PAGE gels, and bands were visualized by fluorescence imaging and quantified using ImageQuant™ (GE Healthcare). Concentrations of RIPtides ranged from 0.6 nM to 60 μM, and for the initial screening, experiments were performed in duplicate using HeLa cell extracts. For active RIPtides, experiments were repeated using DU145 (prostate cancer) and HEK293 cell extracts. Several controls were included in the design of the experiments: a positive control (untreated cell lysate), negative controls (buffer only, heat inactivated and RNase treated cell extracts), and PCR amplification control (60 μM of RIPtide added after telomerase elongation and before PCR step). For cell-based TRAP assays, DU145 cells were transfected with 0.2% Lipofectamine™ 2000 (Invitrogen) and 165 nM RIPtide for a period of 24 h. Cells were harvested, counted, lysed with 1×CHAPS lysis buffer and normalized by total protein concentration as determined by the Bradford assay. Assays were performed in triplicate as described above.

### Microarray Manufacture

For the fabrication of 2′-O-methyl oligonucleotide-based high-density microarrays, a photoresist technique based in I-line (365 nm) projection lithography was utilized¹³. This method differs from that used in the manufacture of Affymetrix Genechip microarrays, which employs 2′-deoxynucleoside phosphoramidites having a photodeprotectable 5′-protecting group. 5′-DMT-2′-O-methyl phosphoramidites were used as monomers for the on-chip synthesis of the RIPtide microarrays, with a photogenerated acid being used to remove the 5′-DMT group during chain extension. The silica substrate for the arrays was first silanized and then reacted with a hexaethyleneglycol derivative (used as a spacer between the oligonucleotides and the array surface) before the initial nucleic acid coupling step. Then, a film containing the photoacid generator was coated onto the substrate, aligned, and exposed in the stepper to the first mask, giving rise to photogenerated acid which allowed the first detritylation. The film was then removed and the substrate processed in a cell flow in which the first DMT-protected phosphoramidite monomer was added. Subsequent steps of capping, oxidation, and washes were carried out, and the process was repeated using the next mask and oligonucleotide in the sequence (FIG. 2). After the synthesis was completed, substrates were treated with a solution of organic base to remove protecting groups from the RIPtides. Wafers were rinsed, spin-dried under nitrogen and diced into individual chips. The final density of full length RIPtide on these microarrays was approx. 30-50 pmol/cm², with a feature size of 17.5 μm. The chips also included a checkerboard for grid alignment consisting of the 13-mer 2′-O-Me sequence 5′-ACGGTAGCATCTT-3′ (SEQ ID NO: 56) which allows hybridization with the commercial Affymetrix Oligo B2 (5′-biotin-GTCAAGATGATGCTACCGTTCAG-3′; (SEQ ID NO: 57)).

### RNA Production

Forward and reverse primers for RNA domain transcription: Full-length hTR, 1-451 nt (5′-GCCAAGCTTTAATACGACTCACTATAGGG-3′(SEQ ID NO: 58), 5′-GCATGTGTGAGCCGAGTCCTGGGTGCACGT-3′(SEQ ID NO: 59)), Pseudoknot/Template, 1-211 nt (same as forward full-length, 5′-GTCCCCGGGAGGGGCGAACGGGCCAGCAGC-3′(SEQ ID NO: 60)), PK123, 63-185 nt (5′-TAATACGACTCACTATAGGGCGTAGGCGCCGTGCTT-TTGCTCCCCGCGCGC3′ (SEQ ID NO: 61), 5′-CAGCTGACATTTTTTGTTTGCTCTAGAATGA-ACGGT-3′ (SEQ ID NO: 62)), PK159, 33-191 nt (5′-TAATACGACTCACTATAGGCCATTTTTT-GTCTAACCCTAACTGAGAAGGGC-3′(SEQ ID NO: 63), 5′-GGCCAGCAGCTGACATTTTTTGT-TTGCTCTAGAATG-3′ (SEQ ID NO: 64)), PK175, 26-100 nt (5′-TAATACGACTCACTATAGG-GTGGTGGCCATTTTTTGTCTAACCCTAACTGA-3′(SEQ ID NO: 65), 5′-GGGCGAACGGGCCAG-CAGCTGACATTTTTTGTTTGC-3′(SEQ ID NO: 66)).

In vitro transcription reagents: Cy3-labeled RNA. Transcription reactions contained 20 μL of 10× transcription buffer, 40 μL NTPs (20 mM, Invitrogen), 10 μL of aminoallyl-UTP (50 mM, Fermentas), 60 μL PCR product, 20 μL IPPase (Aldrich, dissolved to 0.01 U/μL)-RNase inhibitor (Roche), 5 μL of T7-RNA polymerase and 45 μL RNase-free water, for a 200 μL reaction volume. Transcription yield was typically in the range 0.1-0.25 mg RNA per 1 μg of DNA template. Unlabeled RNA. Commonly employed conditions for full-length hTR, for FP experiments: 20 μL 10× transcription buffer, 40 μL NTPs (20 mM, Invitrogen), 60 μL PCR product, 20 μL IPPase (Aldrich, dissolved to 0.01 U/μL)-RNase inhibitor (Roche), 5 μL of T7-RNA polymerase and 55 μL RNase-free water, for a 200 μL reaction, with a typical yield of 0.1-0.25 mg RNA per 1 μg of DNA. Transcription buffer (10×): 400 mM Tris, pH 8, 100 mM MgCl₂, 50 mM DTT, 10 mM spermidine and 0.1% Triton X-100.

### Additional Microarray Protocols

Buffers and reagents: 2× Hybridization buffer (100 mM MES, 1M [Na⁺], 20 mM EDTA, 0.01% Tween 20); 2× staining buffer (100 mM MES, 1M [Na⁺], 0.05% Tween 20); Wash A (6×SSPE, 0.01% Tween 20, 0.005% antifoam); Wash B (100 mM MES, 0.1M [Na⁺], 0.01% Tween 20); 20×SSPE (3M NaCl, 0.2 M NaH₂PO₄, 0.02 M EDTA); SSPE, Saline-Sodium Phosphate-EDTA; MES, 2-(N-morpholino)ethanesulfonic acid; BSA, Bovine serum albumin; SAPE, Streptavidin phycoerythrin

The following procedure is a modification of the Genechip Hybridization Protocols, specially adapted to screen for RIPtide binders employing folded RNA. Checkerboard staining: (1) Hybridization of oligo B2 (Affymetrix Inc.). Hybridization cocktail: oligo B2 (3 nM, final concentration 250 pM), BSA, 2× hybridization buffer, and RNase-free water. Conditions: 16 h, 45° C., 60 rpm, using a GeneChip® hybridization oven 640 (Affymetrix). (2) Staining using Affymetrix protocol FlexGEws2×4v_{—}450, and the following staining cocktail: 2× staining buffer, BSA, SAPE, and RNase free water.

Array conditions: Standard conditions. The RNA target was dissolved in the buffer described in the Methods section and refolded. 100 nM RNA was incubated with the array at 37° C. for 1 h, 60 rpm, inside a GeneChip® hybridization oven. The array was then briefly washed (5 min) with the folding buffer (full washing protocol available upon request). For RNAs larger than 80 nt, the ‘EukGEws1’ protocol from Affymetrix was employed (see below). Other commonly used conditions entailed the incubation of 10 nM of target RNA with the array for 6 h at 37° C. In addition, for the large RNA transcripts PK123 and PK159, incubations at 10 nM for 18 h 37° C. were also tested. These conditions normally resulted in a higher degree of Watson-Crick recognition. Microarray washings: (1) Initial wash (mild). 50 mM potassium phosphate buffer, 5 mM Mg(OAc)₂, 150 mM KCl, pH=7.4. 5 cycles of 3 mixes/cycle at 25° C., with 1× array buffer (˜5 min). This washing protocol was applied to all RNA constructs. (2) Second wash (adapted from Affymetrix Genechip Protocols, more stringent). Additional washing suitable for constructs larger than 80 nt. 10 cycles of 2 mixes/cycle at 25° C., with wash buffer A, 4 cycles of 15 mixes/cycle at 50° C., with wash buffer B, 30 min wash A, and 10 cycles of 4 mixes/cycle at 25° C., with wash buffer A.

### RIPtide Synthesis

2′-OMe RIPtides were prepared using a MerMade 12 (BioAutomation) DNA synthesizer, in a 0.2 or 1 μmol scale using a coupling time of 6 min and an oxidation step of 50 seconds. The syntheses were carried out DMT-on for subsequent Poly Pak-II (Glen Research) purification. Selected RIPtides were further purified by C18-reverse phase HPLC for use in activity assays. For phosphorothioate and LNA syntheses, the same parameters were used, using sulfurizing reagent II (DDTT) and LNA phosphoramidite monomers, also from Glen Research.

### TRAP Assays

The inhibitory potential of the RIPtides was initially assessed in HeLa cell extracts, in duplicate experiments, using a 600 pM-60 μM concentration range. Experiments with selected RIPtides were repeated for a concentration range of 0.6 pM-60 μM. All RIPtides reported here were 2′-O-methyl derivatives (with phosphodiester or phosphorothioate backbone), with the exception of sequence IV-3, which was also synthesized and assayed as an all-LNA sequence. RIPtide length varied from 6 to 8 nucleotides (hits from the RIPtide microarray screen) and, in addition, a series of 12-mers and 14-mers were studied for each cluster of interest in order to determine the effect of RIPtide length on their potency as telomerase inhibitors.

### Cell Culturing Conditions

The transformed embryonic kidney cell line HEK293 and the prostate cancer cell line DU145 were maintained in DMEM supplemented with 10% fetal bovine serum in 5% CO₂ at 37° C. Soluble cell extracts for TRAP assays were prepared by detergent lysis of 10⁶ cells with 200 μL 1×CHAPS Lysis Buffer (Chemicon) as described in the manufacturer's instructions.

### SUMMARY

Described herein is a novel, structurally unbiased microarray-based method for the identification of short polynucleotides that target folded RNA molecules, referred to herein as RIPtides, for RNA-Interacting Polynucleotides. The key component of the platform is an N-mer microarray presenting all possible sequences of 2′-O-methylated RNA having between 4 and 8 nucleotides in length (N=4, 5, 6, 7, and 8) and bearing the four canonical RNA bases (A, C, G, and U). This report represents the first employing a large, high-density microarray of any nucleic acid analog.

It was found that 2′-O-methyl RIPtides typically bind their targets greater than 50-fold more tightly than the corresponding 2′-deoxyoligonucleotides. It was also found that N-mer RIPtide microarrays comprising all 2′-oligodeoxynucleotides of N=4-8 required micromolar concentrations of the RNA target and overnight incubations in order to observe hits, and these were virtually all 8-mers (W. L. S., A. R. P., R. K., G. M., and G. L. V., unpublished results). By contrast, with 2′-O-methylated RIPtide microarrays, incubations of 1 hour with nanomolar concentrations of RNA yielded significant numbers of hits, with 8-mers, 7-mers and even 6-mer hits being represented and subsequently validated as binders in solution. The photoresist-based synthesis procedure employed here, which is fully compatible with commercially available 5′-dimethoxytrityl-protected 3′-phosphoramidites, should be immediately applicable, for example, to the fabrication of RIPtide microarrays presenting many other varieties of potentially interesting and useful nucleic acid analogs. The possibilities for nucleic acid analogs, include but are not limited to locked nucleic acids (LNAs) (Kaur, H. et al., Chem. Rev. 107, 4672-4697 (2007)), 2′-methoxyethyl-(MOE) substituted RNAs (Bennett, C. F., Antisense Drug Technology (2nd Ed.), 273-303 (2008)), and glycidyl nucleic acids (GNAs) (Schlegel, M. K. et al., ChemBioChem 8, 927-932 (2007)).

Though the microarray screen was devised to be unbiased with respect to canonical Watson-Crick binding versus non-canonical modes of interactions, in the present screens no clear example of a non-canonical binder. It is entirely possible that a more exhaustive analysis of a much greater number of hits would yield non-canonical binders, but at least with the telomerase pseudoknot, the top 20-30 always showed near-complete Watson-Crick complementarity to a sequence on the target RNA, and these hits formed a cluster with others having slight frame-shifts with respect to the target or other minor differences in sequence or length. One important feature of intramolecular RNA/RNA interactions (i.e., RNA folding) is the 2′-hydroxyl group, which frequently engages in a wide and varied array of hydrogen-bonding interactions (Leontis, N. B, Westhof, E., RNA 7, 499-512 (2001)). It could be, without wishing to be bound by theory, that these interactions involving the 2′-OH provide a stabilizing force that is indispensible for the formation of non-canonical bound structures. This can be tested, for example, by fabricating microarrays having a 2′-hydroxyl or a functional equivalent. In another embodiment, the alphabet of nucleobases represented in RIPtide arrays can be expanded to include those with substantial propensity to pair in Hoogsteen or other modes; examples of such nucleobases include, but are not limited to, 8-oxo- and 8-amino derivatives of guanine and adenine.

The RIPtide screening experiments reported herein have identified four regions on the telomerase pseudoknot/template region that are available for binding short 2′-O-methylated polynucleotides. Of these regions, the one that bound the largest number of RIPtides (Cluster V) is the template. That the template engages microarray-bound RIPtides provides a validation for the method as a screen for especially productive binding sites in a folded RNA target. The observation that so few sites on the RNA turn out to be targetable by RIPtides, and that all the sites identified in the present screens are known from structural probing and sequence covariation to have at least partial single-stranded character, provide further evidence that the RNA target adopts a folded structure related to that depicted in folding diagrams. That said, certain regions in the pseudoknot/template that might be predicted on the basis of secondary structure alone to be accessible turn out not to be productive for RIPtide binding. For example, the J2a.1/2a bubble, the 5′- and 3′-ends of the template, and the entire 3′-end of the J2a/3 loop are barely targeted if at all in PK159 (FIG. 4C), suggesting that these regions may not be as free of pairing interactions as suggested by two-dimensional folding diagrams. High-resolution structures of folded RNA molecules have revealed that regions suggested by folding diagrams to be single-stranded are often in fact paired, frequently via non-canonical interactions. It is noted that although the regions targeted by Clusters II, III and IV are predicted to be partially single-stranded, in each case the targeted region extends into an adjacent segment believed to form a Watson-Crick duplex, and in several instances the cluster preferentially migrates into the adjacent duplex in preference to engaging an adjacent segment of the same loop. RIPtide binding events that involve strand displacement might be characterized by on-rates that are slower than those for freely accessible sites. It is envisioned that determining on-rates can yield valuable insights. Without wishing to be bound or constrained by theory, the correlation observed between solution K_{d} values and rank order of the microarray hits might result from non-uniformity in binding kinetics among the members of the arrayed RIPtide library.

The approach followed here, namely RIPtide microarray screening of isolated RNA elements from a large ribonucleoprotein particle, has significant advantages over current methods in the art. The most significant advantages are that RNAs in the optimal range for RIPtide microarray screening, those below ˜160 nt, are easy to obtain and often fold into a stable structure. With respect to telomerase, one possibility is that targeting the RNA alone will inhibit telomerase activity by preventing RNP assembly, which can be tested, for example, by blocking binding of the accessory subunit dyskerin via targeting the ScaRNA domain of hTR. As described herein, using this strategy followed by efficacy optimization, novel sequences, including, but not limited to, SEQ ID NO:1 and SEQ ID NO:20, that inhibit human telomerase activity in vitro and in vivo were identified.

The novel method does not require a previous structural characterization of the RNA target and allows the mapping of a well-structured RNA for the identification of preferential binding sites to short oligonucleotides. Short oligonucleotides are likely to exhibit better drug-like characteristics than longer oligonucleotides, such as improved cellular uptake, ease of preparation and modification at reduced costs, etc., while still retaining high affinity for RNA. For these oligonucleotide-based drugs, the assumption is that net negative charge is an impediment for oligonucleotide cellular uptake, so it was envisioned that relatively shorter RIPtides carrying a reduced negative charge due to the fewer phosphate groups would display better cell permeability profiles than traditional 20-mer oligonucleotides utilized in other RNA-related targeting approaches. At the same time, the requirement for short sequences considerably simplifies the manufacturing process of the microarrays, making possible the incorporation of different sizes and chemical modifications in a custom-format array, not to mention the overall reduction in time and cost of synthesis.

In initial efforts, microarrays were employed consisting of 2′-O-methyl RIPtides, but the same methodology could be applied using other nucleotide-based molecules (such as Glycol Nucleic Acids, homo DNA, RIPtides with modifications at the bases, sugar, backbone, etc.). Furthermore, the approach is not limited to a single microarray platform. Although the initial application of the RIPtide approach was to microarrays manufactured by Affymetrix in a similar format to the high-density Genechip array, the concept could also be extended to different type of arrays, e.g. home-made microarrays, as long as the synthesized RIPtides can be immobilized onto a solid surface.

Another aspect of interest, which is distinct in the RIPtide microarrays approach, is the fact that, in principle, and taking into account the relevant role of non-canonical interactions in the process of RNA folding and RNA-protein recognition events, the screening of folded RNA in the presence of RIPtides could provide a way for identification of RNA binders not limited exclusively to Watson-Crick recognition events. Thus, an unbiased or rule-free screening was designed to be able to detect the full repertoire of oligonucleotide-RNA interactions, which include both canonical (Watson-Crick base pairing) as well as putative non canonical (Wobble, Hoogsteen, sheared pairs, etc) interactions.

In the present study, the RIPtide methodology was applied for the study of a domain of a highly structured RNA belonging to a rather complex biological system, the human ribonucleoprotein telomerase, but other RNAs could be used as targets as well. In the case of the human telomerase pseudoknot/template domain, and for the particular case of 2′-O-methyl RIPtides, a higher propensity for oligonucleotide binding was found to the template region of the pseudoknot/template domain, which is known to be very accessible in a cellular context, the loop J2a/J2b, loop J2b/3 (also suggesting that the pseudoknot may not be permanently formed under our experimental in vitro conditions) and the 5′ end of loop J2a/3. Most of these regions comprise loops and fragments of sequence predicted to be relatively open in the RNA structure, in the absence of other protein components.

In a biological context, as hTR is expected to be fully associated with the transcriptase and different proteins in cells as a constituent of the holoenzyme RNP complex, it is conceivable that part of the RNA will be in close interaction with different protein components not included in our screening studies, which could reduce access of the RIPtides for optimal interaction with hTR. However, the RIPtide screening has already facilitated the identification of several sequences with significant anti-telomerase activity. It is predicted that this technology could be used as a tool to expedite the discovery of many other novel nucleic-acid sequences that can be used as modulators of telomerase function by interfering with catalysis and/or assembly, by screening other functional and structural domains within hTR.

The present invention can be defined in any of the following numbered paragraphs:
1. A telomerase inhibitor, the telomerase inhibitor comprising a nucleic acid or analog thereof, which binds to the CR4-CR5 domain of the RNA component of human telomerase.
2. The telomerase inhibitor of paragraph 1, wherein said nucleic acid is a ribonucleic acid.
3. The telomerase inhibitor of paragraph 1, wherein said nucleic acid is a nucleic acid analog.
4. The nucleic acid analog of paragraph 3, wherein said nucleic acid analog is a ribonucleic acid analog.
5. The telomerase inhibitor of paragraph 1, wherein said telomerase inhibitor binds to the J5/J6 loop of said CR4-CR5 domain.
6. The telomerase inhibitor of paragraph 1, wherein said nucleic acid or analog thereof comprises a binding sequence length of 4-20 nucleotides.
7. The telomerase inhibitor of paragraph 1, wherein said telomerase inhibitor comprises, or alternatively consists essentially of, or as a further alternative, consists of, a sequence selected the group consisting of SEQ ID NO: 1-SEQ. ID NO: 10.
8. The telomerase inhibitor of paragraph 1, wherein said telomerase inhibitor comprises, or alternatively consists essentially of, or as a further alternative, consists of, a sequence selected the group consisting of SEQ ID NO: 1 and SEQ ID NO: 2.
9. A method of inhibiting telomerase activity, the method comprising contacting a telomerase with a nucleic acid or analog thereof, which binds to the CR4-CR5 domain of the RNA component of human telomerase.
10. The method of paragraph 9, wherein said nucleic acid is a ribonucleic acid.
11. The method of paragraph 9, wherein said nucleic acid is a nucleic acid analog.
12. The nucleic acid analog of paragraph 11, wherein said nucleic acid analog is a ribonucleic acid analog.
13. The method of paragraph 9, wherein said telomerase inhibitor binds to the J5/J6 loop of said CR4-CR5 domain.
14. The method of paragraph 9, wherein said nucleic acid or analog thereof comprises a binding sequence length of 4-20 nucleotides.
15. The method of paragraph 9, wherein said nucleic acid or analog thereof comprises, or alternatively consists essentially of, or as a further alternative, consists of, a sequence selected from the group consisting of SEQ ID NO: 1-SEQ. ID NO: 10.
16. The method of paragraph 9, wherein said nucleic acid or analog thereof comprises, or alternatively consists essentially of, or as a further alternative, consists of, a sequence selected from the group consisting of SEQ ID NO: 1 and SEQ ID NO: 2.
17. A method of inhibiting telomerase activity in a cell, the method comprising contacting a cell with a nucleic acid or analog thereof, which binds to the CR4-CR5 domain of the RNA component of human telomerase.
18. The method of paragraph 17, wherein said cell is contacted in vitro.
19. The method of paragraph 17, wherein said nucleic acid is a ribonucleic acid.
20. The method of paragraph 17, wherein said nucleic acid is a nucleic acid analog.
21. The nucleic acid analog of paragraph 20, wherein said nucleic acid analog is a ribonucleic acid analog.
22. The method of paragraph 17, wherein said telomerase inhibitor binds to the J5/J6 loop of said CR4-CR5 domain.
23. The method of paragraph 17, wherein said nucleic acid or analog thereof comprises a binding sequence length of 4-20 nucleotides.
24. The method of paragraph 17, wherein said nucleic acid or analog thereof comprises, or alternatively consists essentially of, or as a further alternative, consists of, a sequence selected from the group consisting of SEQ ID NO: 1-SEQ. ID NO: 10.
25. The method of paragraph 17, wherein said nucleic acid or analog thereof comprises, or alternatively consists essentially of, or as a further alternative, consists of, a sequence selected from the group consisting of SEQ ID NO: 1 and SEQ ID NO: 2.
26. A method of treating a proliferative disorder in a subject in need thereof, the method comprising administering to the subject an effective amount of a telomerase inhibitor, wherein said telomerase inhibitor comprises a nucleic acid or analog thereof, which binds to the CR4-CR5 domain of the RNA component of human telomerase.
27. The method of paragraph 26, wherein said nucleic acid is a ribonucleic acid.
28. The method of paragraph 26, wherein said nucleic acid is a nucleic acid analog.
29. The nucleic acid analog of paragraph 28, wherein said nucleic acid analog is a ribonucleic acid analog.
30. The method of paragraph 26, wherein the telomerase inhibitor binds to the J5/J6 loop of said CR4-CR5 domain.
31. The method of paragraph 26, wherein said nucleic acid or analog thereof comprises a binding sequence length of 4-20 nucleotides.
32. The method of paragraph 26, wherein said telomerase inhibitor comprises, or alternatively consists essentially of, or as a further alternative, consists of, a sequence selected from the group consisting of SEQ ID NO: 1-SEQ. ID NO: 10.
33. The method of paragraph 26, wherein said telomerase inhibitor comprises, or alternatively consists essentially of, or as a further alternative, consists of, a sequence selected from the group consisting of SEQ ID NO: 1 and SEQ ID NO: 2.
34. The method of paragraph 26, wherein said proliferative disorder is a cancer.
35. A therapeutic composition comprising a telomerase inhibitor and a pharmaceutically acceptable carrier, wherein said telomerase inhibitor comprises a nucleic acid or analog thereof, which binds to the CR4-CR5 domain of the RNA component of human telomerase.
36. The therapeutic composition of paragraph 35, wherein said nucleic acid is a ribonucleic acid.
37. The therapeutic composition of paragraph 35, wherein said nucleic acid is a nucleic acid analog.
38. The nucleic acid analog of paragraph 37, wherein said nucleic acid analog is a ribonucleic acid analog.
39. The therapeutic composition of paragraph 35, wherein the telomerase inhibitor binds to the J5/J6 loop of said CR4-CR5 domain.
40. The therapeutic composition of paragraph 35, wherein said nucleic acid or analog thereof comprises a binding sequence length of 4-20 nucleotides.
41. The therapeutic composition of paragraph 35, wherein said telomerase inhibitor comprises, or alternatively consists essentially of, or as a further alternative, consists of, a sequence selected from the group consisting of SEQ ID NO: 1-SEQ. ID NO: 10.
42. The therapeutic composition of paragraph 35, wherein said telomerase inhibitor comprises, or alternatively consists essentially of, or as a further alternative, consists of, a sequence selected from the group consisting of SEQ ID NO: 1 and SEQ ID NO: 2.
43. A telomerase inhibitor, the inhibitor comprising a nucleic acid molecule or analog thereof, which binds to the pseudoknot/template domain of the RNA component of human telomerase, wherein said nucleic acid molecule or analog thereof comprises, or alternatively consists essentially of, or as a further alternative, consists of, a binding sequence selected from the group consisting of SEQ ID NO: 11-SEQ. ID NO: 45.
44. The telomerase inhibitor of paragraph 43, wherein said binding sequence comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45.
45. The telomerase inhibitor of paragraph 43, wherein said binding sequence comprises, or alternatively consists essentially of, or as a further alternative, consists of, SEQ. ID NO: 20.
46. A method of inhibiting telomerase activity in a cell, the method comprising contacting a cell with a ribonucleic acid molecule or analog thereof, which binds to the pseudoknot/template domain of the RNA component of human telomerase, wherein said ribonucleic acid molecule or analog thereof comprises, or alternatively consists essentially of, or as a further alternative, consists of, a binding sequence selected from the group consisting of SEQ ID NO: 11-SEQ. ID NO: 45.
47. The method of paragraph 46, wherein said binding sequence comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45.
48. The method of paragraph 46, wherein said binding sequence comprises, or alternatively consists essentially of, or as a further alternative, consists of, SEQ. ID NO: 20.
49. A method of treating a proliferative disorder in a subject in need thereof, the method comprising administering to the subject an effective amount of a telomerase inhibitor, wherein said telomerase inhibitor comprises a ribonucleic acid molecule or analog thereof, which binds to the pseudoknot/template domain of the RNA component of human telomerase, wherein said wherein said ribonucleic acid molecule or analog thereof comprises, or alternatively consists essentially of, or as a further alternative, consists of, a binding sequence selected from the group consisting of SEQ ID NO: 11-SEQ. ID NO: 45.
50. The method of paragraph 49, wherein said binding sequence comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45.
51. The method of paragraph 49, wherein said binding sequence comprises, or alternatively consists essentially of, or as a further alternative, consists of, SEQ. ID NO: 20.
52. The method of paragraph 49, wherein said proliferative disorder is a cancer.
53. A therapeutic composition comprising a telomerase inhibitor and a pharmaceutically acceptable carrier, wherein said telomerase inhibitor comprises a nucleic acid or analog thereof, which binds to the pseudoknot/template domain of the RNA component of human telomerase, wherein said wherein said ribonucleic acid molecule or analog thereof comprises, or alternatively consists essentially of, or as a further alternative, consists of, a binding sequence selected from the group consisting of SEQ ID NO: 11-SEQ. ID NO: 45.
54. The therapeutic composition of paragraph 49, wherein said binding sequence comprises, or alternatively consists essentially of, or as a further alternative, consists of, a sequence selected from the group consisting of SEQ ID NO: 19-SEQ ID NO: 24; SEQ ID NO: 39; SEQ ID NO: 44; and SEQ ID NO: 45.
55. The therapeutic composition of paragraph 49, wherein said binding sequence comprises, or alternatively consists essentially of, or as a further alternative, consists of, SEQ. ID NO: 20.

### TABLES

TABLE 1

| | Consensus | |
|---|---|---|
| Cluster | Sequence | SEQ ID NO: |
| II | XAGCGAX | SEQ ID NO: 46 |
| III | XGGAGCAX | SEQ ID NO: 47 |
| IV | GAAGGCG | SEQ ID NO: 48 |
| IV | GAACGGUG | SEQ ID NO: 49 |
| V | XGGUUAAGX | SEQ ID NO: 50 |
| V | AGUUAGG | SEQ ID NO: 51 |

TABLE 2

| SEQ ID NO: 1 | | IC₅₀ from | |
|---|---|---|---|
| Variant | Kₚ from FP | TRAP | |
| G*CCUCCAG | 8.8 ± 2.5 nM | ~9 μM | |
| GCCUCCA*G | 8.9 ± 5.6 nM | ~16 μM | Phospho- |
| | | | rothioate |
| G*CCUCCA*G | 37.3 ± 15.2 nM | — | |
| G*C*C*U*C*C*A*G | ND | — | |
| G*C*C*U*C*C*A*G | ND | ~6 μM | LNA |

1. A telomerase inhibitor, the telomerase inhibitor comprising a nucleic acid or analog thereof, which binds to the CR4-CR5 domain of the RNA component of human telomerase.

2. The telomerase inhibitor of claim 1, wherein said nucleic acid is a ribonucleic acid.

3-4. (canceled)

5. The telomerase inhibitor of claim 1, wherein said telomerase inhibitor binds to the J5/J6 loop of said CR4-CR5 domain.

6. The telomerase inhibitor of claim, wherein said nucleic acid or analog thereof comprises a binding sequence length of 4-20 nucleotides.

7. The telomerase inhibitor of claim 1, wherein said telomerase inhibitor comprises a sequence selected the group consisting of SEQ ID NO: 1-SEQ. ID NO: 10.

8. (canceled)

9. A method of inhibiting telomerase activity, the method comprising contacting a telomerase with a nucleic acid or analog thereof, which binds to the CR4-CR5 domain of the RNA component of human telomerase.

10. The method of claim 9, wherein said nucleic acid is a ribonucleic acid.

11-12. (canceled)

13. The method of claim 9, wherein said telomerase inhibitor binds to the J5/J6 loop of said CR4-CR5 domain.

14. The method of claim 9, wherein said nucleic acid or analog thereof comprises a binding sequence length of 4-20 nucleotides.

15. The method of claim 9, wherein said nucleic acid or analog thereof comprises a sequence selected from the group consisting of SEQ ID NO: 1-SEQ. ID NO: 10.

16-25. (canceled)

26. A method of treating a proliferative disorder in a subject in need thereof, the method comprising administering to the subject an effective amount of a telomerase inhibitor, wherein said telomerase inhibitor comprises a nucleic acid or analog thereof, which binds to the CR4-CR5 domain of the RNA component of human telomerase.

27. The method of claim 26, wherein said nucleic acid is a ribonucleic acid.

28-29. (canceled)

30. The method of claim 26, wherein the telomerase inhibitor binds to the J5/J6 loop of said CR4-CR5 domain.

31. The method of claim 26, wherein said nucleic acid or analog thereof comprises a binding sequence length of 4-20 nucleotides.

32. The method of claim 26, wherein said telomerase inhibitor comprises a sequence selected from the group consisting of SEQ ID NO: 1-SEQ. ID NO: 10.

33-34. (canceled)

35. The telomerase inhibitor of claim 1, further comprising a pharmaceutically acceptable carrier.

36-42. (canceled)

43. A telomerase inhibitor, the inhibitor comprising a nucleic acid molecule or analog thereof, which binds to the pseudoknot/template domain of the RNA component of human telomerase, wherein said nucleic acid molecule or analog thereof comprises a binding sequence selected from the group consisting of SEQ ID NO: 11-SEQ. ID NO: 45.

44. The telomerase inhibitor of claim 43, wherein said binding sequence is selected from the group consisting of SEQ ID NO: 19-SEQ ID NO: 24; SEQ ID NO: 39; SEQ ID NO: 44; and SEQ ID NO: 45

45. (canceled)

46. A method of inhibiting telomerase activity in a cell, the method comprising contacting a cell with a ribonucleic acid molecule or analog thereof, which binds to the pseudoknot/template domain of the RNA component of human telomerase, wherein said ribonucleic acid molecule or analog thereof comprises a binding sequence selected from the group consisting of SEQ ID NO: 11-SEQ. ID NO: 45.

47-52. (canceled)

53. The telomerase inhibitor of claim 43, further comprising a pharmaceutically acceptable carrier.

54-55. (canceled)


SEQUENCE LISTING (71 sequences)

SEQ ID NO: 1 - RNA, 8 nt, Artificial Sequence
  Description of Artificial Sequence Synthetic oligonucleotide
gccuccag

SEQ ID NO: 2 - DNA, 8 bp, Artificial Sequence
  Description of Artificial Sequence Synthetic oligonucleotide
gcctccag

SEQ ID NO: 3 - RNA, 8 nt, Artificial Sequence
  Description of Artificial Sequence Synthetic oligonucleotide
gccuccau

SEQ ID NO: 4 - RNA, 8 nt, Artificial Sequence
  Description of Artificial Sequence Synthetic oligonucleotide
gccuccua

SEQ ID NO: 5 - RNA, 8 nt, Artificial Sequence
  Description of Artificial Sequence Synthetic oligonucleotide
gccucccc

SEQ ID NO: 6 - RNA, 7 nt, Artificial Sequence
  Description of Artificial Sequence Synthetic oligonucleotide
gccucca

SEQ ID NO: 7 - RNA, 6 nt, Artificial Sequence
  Description of Artificial Sequence Synthetic oligonucleotide
gccucc

SEQ ID NO: 8 - RNA, 8 nt, Artificial Sequence
  Description of Artificial Sequence Synthetic oligonucleotide
gccuccaa

SEQ ID NO: 9 - RNA, 8 nt, Artificial Sequence
  Description of Artificial Sequence Synthetic oligonucleotide
gcccaacu

SEQ ID NO: 10 - DNA, 8 bp, Artificial Sequence
  Description of Artificial Sequence Synthetic oligonucleotide
gcccaact

SEQ ID NO: 11 - RNA, 8 nt, Artificial Sequence
  Description of Artificial Sequence Synthetic oligonucleotide
gucagcga

SEQ ID NO: 12 - RNA, 8 nt, Artificial Sequence
  Description of Artificial Sequence Synthetic oligonucleotide
agcgagaa

SEQ ID NO: 13 - RNA, 12 nt, Artificial Sequence
  Description of Artificial Sequence Synthetic oligonucleotide
gucagcgagaaa

SEQ ID NO: 14 - RNA, 6 nt, Artificial Sequence
  Description of Artificial Sequence Synthetic oligonucleotide
ggagca

SEQ ID NO: 15 - RNA, 8 nt, Artificial Sequence
  Description of Artificial Sequence Synthetic oligonucleotide
ggagcaaa

SEQ ID NO: 16 - RNA, 12 nt, Artificial Sequence
  Description of Artificial Sequence Synthetic oligonucleotide
ggagcaaaagca

SEQ ID NO: 17 - RNA, 10 nt, Artificial Sequence
  Description of Artificial Sequence Synthetic oligonucleotide
ggagcaaaag

SEQ ID NO: 18 - RNA, 10 nt, Artificial Sequence
  Description of Artificial Sequence Synthetic oligonucleotide
gggagcaaaa

SEQ ID NO: 19 - RNA, 8 nt, Artificial Sequence
  Description of Artificial Sequence Synthetic oligonucleotide
gaacggug

SEQ ID NO: 20 - RNA, 10 nt, Artificial Sequence
  Description of Artificial Sequence Synthetic oligonucleotide
gguggaaggc

SEQ ID NO: 21 - RNA, 14 nt, Artificial Sequence
  Description of Artificial Sequence Synthetic oligonucleotide
gaacgguggaaggc

SEQ ID NO: 22 - RNA, 12 nt, Artificial Sequence
  Description of Artificial Sequence Synthetic oligonucleotide
acgguggaaggc

SEQ ID NO: 23 - RNA, 8 nt, Artificial Sequence
  Description of Artificial Sequence Synthetic oligonucleotide
gguggaag

SEQ ID NO: 24 - RNA, 9 nt, Artificial Sequence
  Description of Artificial Sequence Synthetic oligonucleotide
gguggaagg

SEQ ID NO: 25 - RNA, 8 nt, Artificial Sequence
  Description of Artificial Sequence Synthetic oligonucleotide
aggguuag

SEQ ID NO: 26 - RNA, 7 nt, Artificial Sequence
  Description of Artificial Sequence Synthetic oligonucleotide
aguuagg

SEQ ID NO: 27 - RNA, 13 nt, Artificial Sequence
  Description of Artificial Sequence Synthetic oligonucleotide
gucagcgagaaaa

SEQ ID NO: 28 - RNA, 8 nt, Artificial Sequence
  Description of Artificial Sequence Synthetic oligonucleotide
cagcgaga

SEQ ID NO: 29 - RNA, 8 nt, Artificial Sequence
  Description of Artificial Sequence Synthetic oligonucleotide
gacagcgc

SEQ ID NO: 30 - RNA, 8 nt, Artificial Sequence
  Description of Artificial Sequence Synthetic oligonucleotide
cagcgagg

SEQ ID NO: 31 - RNA, 8 nt, Artificial Sequence
  Description of Artificial Sequence Synthetic oligonucleotide
acagcgag

SEQ ID NO: 32 - RNA, 8 nt, Artificial Sequence
  Description of Artificial Sequence Synthetic oligonucleotide
aacagcgc

SEQ ID NO: 33 - RNA, 7 nt, Artificial Sequence
  Description of Artificial Sequence Synthetic oligonucleotide
cagcgag

SEQ ID NO: 34 - RNA, 8 nt, Artificial Sequence
  Description of Artificial Sequence Synthetic oligonucleotide
ucagcgag

SEQ ID NO: 35 - RNA, 8 nt, Artificial Sequence
  Description of Artificial Sequence Synthetic oligonucleotide
acagcgca

SEQ ID NO: 36 - RNA, 8 nt, Artificial Sequence
  Description of Artificial Sequence Synthetic oligonucleotide
agucagcg

SEQ ID NO: 37 - RNA, 8 nt, Artificial Sequence
  Description of Artificial Sequence Synthetic oligonucleotide
aacagcgc

SEQ ID NO: 38 - RNA, 7 nt, Artificial Sequence
  Description of Artificial Sequence Synthetic oligonucleotide
acagcgc

SEQ ID NO: 39 - RNA, 7 nt, Artificial Sequence
  Description of Artificial Sequence Synthetic oligonucleotide
gaaggcg

SEQ ID NO: 40 - RNA, 10 nt, Artificial Sequence
  Description of Artificial Sequence Synthetic oligonucleotide
gggagcaaaa

SEQ ID NO: 41 - RNA, 12 nt, Artificial Sequence
  Description of Artificial Sequence Synthetic oligonucleotide
gcgggagcaaaa

SEQ ID NO: 42 - RNA, 7 nt, Artificial Sequence
  Description of Artificial Sequence Synthetic oligonucleotide
gaaggcg

SEQ ID NO: 43 - RNA, 10 nt, Artificial Sequence
  Description of Artificial Sequence Synthetic oligonucleotide
gguggaaggc

SEQ ID NO: 44 - RNA, 10 nt, Artificial Sequence
  Description of Artificial Sequence Synthetic oligonucleotide
cgguggaagg

SEQ ID NO: 45 - RNA, 11 nt, Artificial Sequence
  Description of Artificial Sequence Synthetic oligonucleotide
gaacgguggaa

SEQ ID NO: 46 - DNA, 7 bp, Artificial Sequence
  Description of Artificial Sequence Synthetic oligonucleotide
nagcgan

SEQ ID NO: 47 - DNA, 8 bp, Artificial Sequence
  Description of Artificial Sequence Synthetic oligonucleotide
nggagcan

SEQ ID NO: 48 - RNA, 7 nt, Artificial Sequence
  Description of Artificial Sequence Synthetic oligonucleotide
gaaggcg

SEQ ID NO: 49 - RNA, 8 nt, Artificial Sequence
  Description of Artificial Sequence Synthetic oligonucleotide
gaacggug

SEQ ID NO: 50 - DNA, 9 bp, Artificial Sequence
  Description of Artificial Sequence Synthetic oligonucleotide
ngguuaagn

SEQ ID NO: 51 - RNA, 7 nt, Artificial Sequence
  Description of Artificial Sequence Synthetic oligonucleotide
aguuagg

SEQ ID NO: 52 - RNA, 10 nt, Artificial Sequence
  Description of Artificial Sequence Synthetic oligonucleotide
ggugcaaggc

SEQ ID NO: 53 - RNA, 10 nt, Artificial Sequence
  Description of Artificial Sequence Synthetic oligonucleotide
ggugccaggc

SEQ ID NO: 54 - RNA, 10 nt, Artificial Sequence
  Description of Artificial Sequence Synthetic oligonucleotide
gcugcaacgc

SEQ ID NO: 55 - protein, 6 aa, Artificial Sequence
  Description of Artificial Sequence Synthetic 6xHis tag
HHHHHH

SEQ ID NO: 56 - DNA, 13 bp, Artificial Sequence
  Description of Artificial Sequence Synthetic oligonucleotide
acggtagcatctt

SEQ ID NO: 57 - DNA, 23 bp, Artificial Sequence
  Description of Artificial Sequence Synthetic oligonucleotide
gtcaagatgatgctaccgttcag

SEQ ID NO: 58 - DNA, 29 bp, Artificial Sequence
  Description of Artificial Sequence Synthetic primer
gccaagctttaatacgactcactataggg

SEQ ID NO: 59 - DNA, 30 bp, Artificial Sequence
  Description of Artificial Sequence Synthetic primer
gcatgtgtgagccgagtcctgggtgcacgt

SEQ ID NO: 60 - DNA, 30 bp, Artificial Sequence
  Description of Artificial Sequence Synthetic primer
gtccccgggaggggcgaacgggccagcagc

SEQ ID NO: 61 - DNA, 51 bp, Artificial Sequence
  Description of Artificial Sequence Synthetic primer
taatacgactcactatagggcgtaggcgccgtgcttttgctccccgcgcgc

SEQ ID NO: 62 - DNA, 36 bp, Artificial Sequence
  Description of Artificial Sequence Synthetic primer
cagctgacattttttgtttgctctagaatgaacggt

SEQ ID NO: 63 - DNA, 51 bp, Artificial Sequence
  Description of Artificial Sequence Synthetic primer
taatacgactcactataggccattttttgtctaaccctaactgagaagggc

SEQ ID NO: 64 - DNA, 36 bp, Artificial Sequence
  Description of Artificial Sequence Synthetic primer
ggccagcagctgacattttttgtttgctctagaatg

SEQ ID NO: 65 - DNA, 51 bp, Artificial Sequence
  Description of Artificial Sequence Synthetic primer
taatacgactcactatagggtggtggccattttttgtctaaccctaactga

SEQ ID NO: 66 - DNA, 36 bp, Artificial Sequence
  Description of Artificial Sequence Synthetic primer
gggcgaacgggccagcagctgacattttttgtttgc

SEQ ID NO: 67 - RNA, 48 nt, Artificial Sequence
  Description of Artificial Sequence Synthetic oligonucleotide
gggcuguuuuucucgcugacuuucagccccaaacaaaaaaugucagca

SEQ ID NO: 68 - RNA, 48 nt, Artificial Sequence
  Description of Artificial Sequence Synthetic oligonucleotide
gcgcuguuuuucucgcugacuuucagcgccaaacaaaaaaugucagcu

SEQ ID NO: 69 - RNA, 160 nt, Artificial Sequence
  Description of Artificial Sequence Synthetic polynucleotide
ggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggccc

SEQ ID NO: 70 - RNA, 11 nt, Artificial Sequence
  Description of Artificial Sequence Synthetic oligonucleotide
caaucccaauc

SEQ ID NO: 71 - DNA, 451 bp, Homo sapiens
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc